(19) United States
(12) Patent Application Publication
(10) Pub. No.: US 2022/0168389 A1
(43) Pub. Date: Jun. 2, 2022

(54) METHODS OF MAKING CHIMERIC ANTIGEN RECEPTOR-EXPRESSING CELLS

(71) Applicants: Novartis AG, Basel (CH); The Trustees of the University of Pennsylvania, Philadelphia, PA (US)

(72) Inventors: Saba Ghassemi, Philadelphia, PA (US); Michael C. Milone, Cherry Hill, NJ (US); Roderick O'Connor, Philadelphia, PA (US)

(73) Assignees: Novartis AG, Basel (CH); The Trustees of the University of Pennsylvania, Philadelphia, PA (US)

(21) Appl. No.: 17/602,730

(22) PCT Filed: Apr. 10, 2020
(86) PCT No.: PCT/US2020/027734

§ 371 Date: Oct. 8, 2021

Related U.S. Application Data
(60) Provisional application No. 62/833,421, filed on Apr. 12, 2019.

(51) Int. Cl. A61K 38/17; C12N 5/0783; C12N 15/62; C12N 15/86; A61K 35/17; A61P 35/00

(52) U.S. Cl. CPC A61K 38/1774; C12N 5/0636; C12N 15/625; C12N 15/86; A61K 35/17; C12N 2501/2315; C12N 2510/00; C12N 2740/15043; C12N 2500/90; C12N 2501/2307; A61P 35/00

(57) ABSTRACT

The invention provides methods of making immune effector cells (e.g., T cells, NK cells) that can be engineered to express a chimeric antigen receptor (CAR), and compositions and reaction mixtures comprising the same.

### RELATED APPLICATION

This application claims priority to U.S. Ser. No. 62/833,421 filed Apr. 12, 2019, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 31, 2020, is named N2067-7163WO_SL.txt and is 91,064 bytes in size.

### FIELD OF THE INVENTION

The present invention relates generally to methods of making immune effector cells (e.g., T cells or NK cells, e.g., quiescent T cells) engineered to express a Chimeric Antigen Receptor (CAR), and compositions comprising the same.

### BACKGROUND OF THE INVENTION

Adoptive cell transfer (ACT) therapy with T cells, especially with T cells transduced with Chimeric Antigen Receptors (CARs), has shown promise in several hematologic cancer trials. The manufacture of gene-modified T cells is a complex process. There exists a need for methods and processes to improve production of the CAR-expressing cell therapy product, enhance product quality, and maximize the therapeutic efficacy of the product.

### SUMMARY OF THE INVENTION

The present disclosure pertains to methods of making immune effector cells (e.g., T cells or NK cells) that can be engineered to express a CAR, and compositions comprising the same. Also disclosed are methods of using such compositions for treating a disease, e.g., cancer, in a subject.

Without wishing to be bound by theory, CART cell therapy can be improved by limiting CART cell differentiation and maintaining their replicative potential. Methods disclosed herein generally relate, at least in part, to enhancing transduction of quiescent T cells, without relying on pre-action through TCR.

Accordingly, in some embodiments, the present disclosure features a method of making a population of immune cells (e.g., T cells) that express a chimeric antigen receptor (CAR), the method comprising: (i) incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; and (ii) transducing the population of immune cells with a nucleic acid molecule, e.g., a nucleic acid molecule on a lentiviral vector, encoding the CAR, e.g., in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby expressing the CAR. In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL. In some embodiments, step (ii) is performed at a cell concentration of about 1×10⁷ cells/mL.

In some embodiments, the method further comprises (iii) harvesting the population of immune cells for storage (e.g., reformulating the population of immune cells in cryopreservation media) or administration, wherein: (a) step (iii) is performed no later than 48 hours, e.g., no later than 14, 16, 18, 20, 22, 24, 26, 28, 30, or 32 hours after the beginning of step (i), (b) the population of immune cells from step (iii) is not expanded, or is expanded by no more than 10, 20, 30, 40, or 50%, e.g., no more than 10%, compared with the population of immune cells at the beginning of step (i), (c) the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i), and/or (d) the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i). In some embodiments, step (iii) is performed no later than 48 hours, e.g., no later than 14, 16, 18, 20, 22, 24, 26, 28, 30, or 32 hours after the beginning of step (i). In some embodiments, the population of immune cells from step (iii) is not expanded, or is expanded by no more than 10, 20, 30, 40, or 50%, e.g., no more than 10%, compared with the population of immune cells at the beginning of step (i). In some embodiments, the population of immune cells from step (iii) is not expanded, or is expanded by no more than 2 hours, 4 hours, 6 hours, 8 hours, 10 hours, 12 hours, 14 hours, 16 hours, 18 hours, 20 hours, 22 hours, 24 hours, 1.5 days, 2 days, 2.5 days, or 3 days, compared with the population of immune cells at the beginning of step (i). In some embodiments, the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i). In some embodiments, the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i).

In some embodiments, step (i) comprises incubating the population of immune cells (e.g., T cells, e.g., freshly isolated T cells, e.g., freshly isolated quiescent T cells) in a medium that does not comprise serum for at least about 2-6 hours. In some embodiments, step (i) comprises incubating the population of immune cells (e.g., T cells, e.g., freshly isolated T cells, e.g., freshly isolated quiescent T cells) in a medium that does not comprise serum for at least about 2 hours. In some embodiments, step (i) comprises incubating the population of immune cells (e.g., T cells, e.g., freshly isolated T cells, e.g., freshly isolated quiescent T cells) in a medium that does not comprise serum for at least about 4 hours. In some embodiments, step (i) comprises incubating the population of immune cells (e.g., T cells, e.g., freshly isolated T cells, e.g., freshly isolated quiescent T cells) in a medium that does not comprise serum for at least about 6 hours.

In some embodiments, step (i) increases expression of low-density lipoprotein receptor (LDL-R) in the population of immune cells, e.g., increases expression of LDL-R by at least about 20, 40, 60, 80, 100, 500, or 1000% as compared to the expression of LDL-R in the population of immune cells prior to step (i). In some embodiments, step (i) increases expression of a receptor that is involved in endocytosis, e.g., a receptor that is involved in the endocytosis of a lentiviral vector, in the population of immune cells. In some embodiments, the expression of such a receptor is increased by at least about 20, 40, 60, 80, 100, 500, or 1000% as compared to the expression of the receptor in the population of immune cells prior to step (i).

In some embodiments, step (i) increases transduction efficiency of step (ii) by, e.g., at least about 2, 4, 6, 8, 10, or 12-fold, compared with an otherwise similar method without step (i), e.g., as measured by expression of CAR (e.g., the percentage of CAR-expressing cells) in the population of immune cells at the end of step (ii).

In some embodiments, step (ii) comprises transducing the population of immune cells with a nucleic acid molecule, e.g., a nucleic acid molecule on a lentiviral vector, encoding the CAR in a medium comprising serum, e.g., a medium comprising at least about 4, 5, or 6% serum.

In some embodiments, step (ii) comprises transducing the population of immune cells with a lentiviral vector comprising a nucleic acid molecule encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours. In some embodiments, step (ii) comprises transducing the population of immune cells with a lentiviral vector comprising a nucleic acid molecule encoding the CAR in a medium comprising about 50 μM deoxynucleosides for about 14-24 hours. In some embodiments, transducing the population of immune cells in a medium comprising deoxynucleosides, e.g., at least about 40 μM-1.5 mM deoxynucleosides, increases transduction efficiency of step (ii) by, e.g., at least about 1, 2, 3, 4, or 5-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (ii). In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL. In some embodiments, step (ii) is performed at a cell concentration of about 1×10⁷ cells/mL.

In some embodiments, step (ii) is performed in a medium comprising IL-7 (e.g., about 10 ng/mL of IL-7) and/or IL-15 (e.g., about 10 ng/mL of IL-15).

In some embodiments, the population of immune cells is not contacted in vitro with an agent that stimulates a CD3/TCR complex and/or an agent that stimulates a costimulatory molecule, e.g., anti-CD3 antibody and/or anti-CD28 antibody. In some embodiments, the population of immune cells is not expanded ex vivo, or if expanded ex vivo, the expansion is shorter than 6 hours, 8 hours, 10 hours, 12 hours, 14 hours, 16 hours, 18 hours, 20 hours, 22 hours, 24 hours, 1.5 days, 2 days, or 3 days.

In another aspect, featured herein is a method of making a population of immune cells (e.g., T cells) that express a chimeric antigen receptor (CAR), the method comprising: (1) transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby expressing the CAR. In some embodiments, step (1) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL. In some embodiments, step (1) is performed at a cell concentration of about 1×10⁷ cells/mL.

In some embodiments, the method further comprises (2) harvesting the population of immune cells for storage (e.g., reformulating the population of immune cells in cryopreservation media) or administration, wherein: (a) step (2) is performed no later than 30 hours, e.g., no later than 12, 14, 16, 18, 20, 22, 24, 26, or 28 hours after the beginning of step (1), (b) the population of immune cells from step (2) is not expanded, or is expanded by no more than 10, 20, 30, 40, or 50%, e.g., no more than 10%, compared with the population of immune cells at the beginning of step (1), (c) the percentage of naïve cells, e.g., naïve T cells, in the population of immune cells from step (2) is not reduced, or is reduced by no more than 10, 20, or 30%, compared with the percentage of naïve cells, e.g., naïve T cells, in the population of immune cells at the beginning of step (1), and/or (d) the percentage of differentiated cells, e.g., differentiated T cells, e.g., terminally differentiated T cells, e.g., CCR7^{low} T cells, in the population of immune cells from step (2) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (1). In some embodiments, step (2) is performed no later than 30 hours, e.g., no later than 12, 14, 16, 18, 20, 22, 24, 26, or 28 hours after the beginning of step (1). In some embodiments, the population of immune cells from step (2) is not expanded, or is expanded by no more than 10, 20, 30, 40, or 50%, e.g., no more than 10%, compared with the population of immune cells at the beginning of step (1). In some embodiments, the population of immune cells from step (2) is not expanded, or is expanded by no more than 2 hours, 4 hours, 6 hours, 8 hours, 10 hours, 12 hours, 14 hours, 16 hours, 18 hours, 20 hours, 22 hours, 24 hours, 1.5 days, 2 days, 2.5 days, or 3 days, compared with the population of immune cells at the beginning of step (1). In some embodiments, the percentage of naïve cells, e.g., naïve T cells, in the population of immune cells from step (2) is not reduced, or is reduced by no more than 10, 20, or 30%, compared with the percentage of naïve cells, e.g., naïve T cells, in the population of immune cells at the beginning of step (1). In some embodiments, the percentage of differentiated cells, e.g., differentiated T cells, e.g., terminally differentiated T cells, e.g., CCR7^{low} T cells, in the population of immune cells from step (2) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (1).

In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 50 μM deoxynucleosides for about 14-24 hours. In some embodiments, transducing the population of immune cells in a medium comprising deoxynucleosides, e.g., at least about 40 μM-1.5 mM deoxynucleosides, increases transduction efficiency of step (1) by, e.g., at least about 1, 2, 3, 4, or 5-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (1).

In some embodiments, step (1) is performed in a medium comprising IL-7 (e.g., about 10 ng/mL of IL-7) and/or IL-15 (e.g., about 10 ng/mL of IL-15).

In some embodiments, the population of immune cells is not contacted in vitro with an agent that stimulates a CD3/TCR complex and/or an agent that stimulates a costimulatory molecule, e.g., anti-CD3 antibody and/or anti-CD28 antibody. In some embodiments, the population of immune cells is not expanded ex vivo, or if expanded ex vivo, the expansion is shorter than 6 hours, 8 hours, 10 hours, 12 hours, 14 hours, 16 hours, 18 hours, 20 hours, 22 hours, 24 hours, 1.5 days, 2 days, or 3 days.

In some embodiments, the population of cells from step (iii) or step (2), after being administered in vivo, persists longer, expands at a higher level, and/or exhibits anti-tumor activity for a longer period, compared with cells made by an otherwise similar method in which cells are expanded in vitro for at least 6, 7, 8, 9, 10, 11, or 12 days before harvesting.

In some embodiments, provided herein is a method of making a population of immune cells (e.g., T cells) that express a chimeric antigen receptor (CAR), the method comprising: (i) increasing expression of low-density lipoprotein receptor (LDL-R) in a population of immune cells (e.g., T cells, e.g., freshly isolated T cells, e.g., freshly isolated quiescent T cells); and (ii) transducing the population of immune cells with a nucleic acid molecule, e.g., a nucleic acid molecule on a lentiviral vector, encoding the CAR, e.g., in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby expressing the CAR. In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL. In some embodiments, step (ii) is performed at a cell concentration of about 1×10⁷ cells/mL. In some embodiments, step (i) comprises introducing a nucleic acid molecule encoding LDL-R into the population of immune cells. In some embodiments, the nucleic acid molecule encoding LDL-R is a DNA molecule. In some embodiments, the nucleic acid molecule encoding LDL-R is an RNA molecule. In some embodiments, the nucleic acid molecule encoding LDL-R is on a viral vector, e.g., a viral vector chosen from a lentivirus vector, an adenoviral vector, or a retrovirus vector. In some embodiments, the nucleic acid molecule encoding LDL-R is on a non-viral vector. In some embodiments, the nucleic acid molecule encoding LDL-R is on a plasmid. In some embodiments, the nucleic acid molecule encoding LDL-R is not on any vector. In some embodiments, the method further comprises (iii) harvesting the population of immune cells for storage (e.g., reformulating the population of immune cells in cryopreservation media) or administration, wherein: (a) step (iii) is performed no later than 48 hours, e.g., no later than 14, 16, 18, 20, 22, 24, 26, 28, 30, or 32 hours after the beginning of step (i), (b) the population of immune cells from step (iii) is not expanded, or is expanded by no more than 10, 20, 30, 40, or 50%, e.g., no more than 10%, compared with the population of immune cells at the beginning of step (i), (c) the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i), and/or (d) the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i).

In some embodiments, provided herein is a method of making a population of immune cells (e.g., T cells) that express a chimeric antigen receptor (CAR), the method comprising: (i) increasing expression of a receptor involved in endocytosis, e.g., the endocytosis of a lentiviral vector, in a population of immune cells (e.g., T cells, e.g., freshly isolated T cells, e.g., freshly isolated quiescent T cells); and (ii) transducing the population of immune cells with a nucleic acid molecule, e.g., a nucleic acid molecule on a lentiviral vector, encoding the CAR, e.g., in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby expressing the CAR. In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (ii) is performed at a cell concentration of about 1×10⁷ cells/mL. In some embodiments, step (i) comprises introducing a nucleic acid molecule encoding the receptor into the population of immune cells. In some embodiments, the nucleic acid molecule encoding the receptor is a DNA molecule. In some embodiments, the nucleic acid molecule encoding the receptor is an RNA molecule. In some embodiments, the nucleic acid molecule encoding the receptor is on a viral vector, e.g., a viral vector chosen from a lentivirus vector, an adenoviral vector, or a retrovirus vector. In some embodiments, the nucleic acid molecule encoding the receptor is on a non-viral vector. In some embodiments, the nucleic acid molecule encoding the receptor is on a plasmid. In some embodiments, the nucleic acid molecule encoding the receptor is not on any vector. In some embodiments, the method further comprises (iii) harvesting the population of immune cells for storage (e.g., reformulating the population of immune cells in cryopreservation media) or administration, wherein: (a) step (iii) is performed no later than 48 hours, e.g., no later than 14, 16, 18, 20, 22, 24, 26, 28, 30, or 32 hours after the beginning of step (i), (b) the population of immune cells from step (iii) is not expanded, or is expanded by no more than 10, 20, 30, 40, or 50%, e.g., no more than 10%, compared with the population of immune cells at the beginning of step (i), (c) the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i), and/or (d) the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i).

In some embodiments of the aforementioned methods, the CAR comprises an antigen binding domain, a transmembrane domain, and an intracellular signaling domain. In some embodiments, the antigen binding domain binds to an antigen chosen from: CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRCSD, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2. In some embodiments, the antigen binding domain binds to CD19. In some embodiments, the antigen binding domain binds to BCMA. In some embodiments, the antigen binding domain comprises a CDR, VH, VL, scFv or a CAR sequence disclosed herein.

In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8. In some embodiments, the transmembrane domain comprises the amino acid sequence of SEQ ID NO: 6, or an amino acid sequence having at least about 85%, 90%, 95%, or 99% sequence identity thereof. In some embodiments, the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region. In some embodiments, the hinge region comprises the amino acid sequence of SEQ ID NO: 2, 3, or 4, or an amino acid sequence having at least about 85%, 90%, 95%, or 99% sequence identity thereof. In some embodiments, the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FccRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 9 or 10, or an amino acid sequence having at least about 85%, 90%, 95%, or 99% sequence identity thereof. In some embodiments, the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain. In some embodiments, the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises a functional signaling domain derived from 4-1BB. In some embodiments, the costimulatory signaling domain comprises the amino acid sequence of SEQ ID NO: 7, or an amino acid sequence having at least about 85%, 90%, 95%, or 99% sequence identity thereof. In some embodiments, the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta. In some embodiments, the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof). In some embodiments, the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In some embodiments, provided herein is a population of CAR-expressing cells (e.g., autologous or allogeneic CAR-expressing T cells or NK cells) made by a method described herein. In some embodiments, provided herein is a pharmaceutical composition comprising a population of CAR-expressing cells described herein and a pharmaceutically acceptable carrier.

In some embodiments, provided herein is a method of increasing an immune response in a subject, comprising administering a population of CAR-expressing cells described herein or a pharmaceutical composition described herein to the subject, thereby increasing an immune response in the subject. In some embodiments, provided herein is a method of treating a cancer in a subject, comprising administering a population of CAR-expressing cells described herein or a pharmaceutical composition described herein to the subject, thereby treating the cancer in the subject. In some embodiments, provided herein is a method of treating a cancer in a subject, comprising administering a population of immune cells expressing a CAR to the subject, thereby treating the cancer in the subject, wherein the population of immune cells expressing a CAR was obtained by: (i) incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; and (ii) transducing the population of immune cells with a nucleic acid molecule, e.g., a nucleic acid molecule on a lentiviral vector, encoding the CAR, e.g., in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby generating the population of immune cells expressing a CAR. In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL. In some embodiments, step (ii) is performed at a cell concentration of about 1×10⁷ cells/mL.

In some embodiments, provided herein is a method of treating a cancer in a subject, comprising administering a population of immune cells expressing a CAR to the subject, thereby treating the cancer in the subject, wherein the population of immune cells expressing a CAR was obtained by: (1) transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby generating the population of immune cells expressing a CAR. In some embodiments, step (1) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (1) is performed at a cell concentration of about 1×10⁷ cells/mL. In some embodiments, the population of immune cells expressing a CAR was obtained from a third party.

In some embodiments, provided herein is a method of treating a cancer in a subject, comprising: incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; transducing the population of immune cells with a nucleic acid molecule, e.g., a nucleic acid molecule on a lentiviral vector, encoding a CAR, e.g., in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours; thereby generating a population of immune cells expressing a CAR; and administering the population of immune cells expressing a CAR to the subject, thereby treating the cancer in the subject. In some embodiments, the population of immune cells is transduced at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., at a cell concentration of about 1×10⁷ cells/mL. In some embodiments, provided herein is a method of treating a cancer in a subject, comprising: transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, thereby generating a population of immune cells expressing a CAR; and administering the population of immune cells expressing a CAR to the subject, thereby treating the cancer in the subject. In some embodiments, the population of immune cells is transduced at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., at a cell concentration of about 1×10⁷ cells/mL.

In some embodiments, the cancer is a solid cancer. In some embodiments, the cancer is chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

In some embodiments, the cancer is a liquid cancer. In some embodiments, the cancer is chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In some embodiments, the invention pertains to a population of CAR-expressing cells described herein, e.g., a population of CAR-expressing cells manufactured using a method described herein, for use as a medicament. In some embodiments, the invention pertains to a population of CAR-expressing cells described herein, e.g., a population of CAR-expressing cells manufactured using a method described herein, for use in a method of increasing an immune response in a subject. In some embodiments, the invention pertains to a population of CAR-expressing cells described herein, e.g., a population of CAR-expressing cells manufactured using a method described herein, for use in a method of treating a cancer in a subject.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIGS. 1A-1F: FIG. 1A: Freshly isolated, quiescent human T cells were transduced with lentiviral vector encoding infrared fluorescent protein (iRFP) for 24-hours followed by washing and culture in IL-7 (10 ng/mL) and IL-15 (10 ng/mL) for the indicated time interval. iRFP+ cells were quantified by flow cytometry. Data are representative of 10 experiments. FIG. 1B: Representative flow cytometric analysis of fresh quiescent T cells transduced as in FIG. 1A. Naïve, central memory and effector memory T cell subsets were identified following gating on live, CD3+, CD4+ or CD8+ T cells using CD45RO and CCR7 expression. FIG. 1C: Transduction efficiency of day 1 cells, 7 days after activation through their CAR, measured by flow cytometry. FIG. 1D: Schematic of the xenograft model and CART19 cell treatment in NSGs IV-injected with 2×10⁶ NALM6 cells. 3×10⁶ day 1 T cells (˜120×10³ CART19) and 3×10⁶ day 9 CART19 cells, as well as control T cells (UTD) were IV-injected in mice 5-7 days after NALM6 injection (n=10). FIG. 1E: Serial quantification of disease burden by bioluminescence imaging. FIG. 1F: Absolute CAR+ peripheral blood CD45+ T cell counts two weeks after CART19 cell or UTD cell injection measured by flow cytometry and a TruCount assay.

FIGS. 2A-2E: Lentiviral transduction of quiescent T cells using an optimized process yields potent CAR-T cells that display during in vivo engraftment. FIG. 2A: Primary human T cells were serum starved for the indicated time periods, and then cultured in IL-7/IL-15 medium and transduced with iRFP. FIG. 2B: Primary human T cells were cultured in IL-7/IL-15 medium and transduced with iRFP. In parallel, these cells were also supplemented with dNs or serum starved for 6h prior to iRFP transduction. FIG. 2C: Using an experimental design as described in FIG. 1D, day 1 T cells (2×10⁶, 0.7×10⁶, or 0.2×10⁶) and day 9 CART19 (3×10⁶ CAR+) cells were injected into Nalm6-bearing NSG mice. Serial quantification of disease burden by bioluminescence imaging show the disease progression. FIG. 2D: Absolute CAR+ peripheral blood CD45+ T cell counts after CART19 cell or UTD cell injection at indicated time points, measured by flow cytometry and a TruCount assay. FIG. 2E: Copies of vector plasmid in peripheral blood measured by qPCR assay.

FIGS. 3A-3E: Lentiviral vectors effectively transduce non-activated T cell subsets with preference for memory subsets. FIG. 3A: Transduction efficiency of freshly isolated human T cells cultured either in IL-7 (10 ng/mL) and IL-15 (10 ng/mL) or activated with beads coated with anti-CD3/CD28 antibody, and transduced with lentiviral vector encoding iRFP for 5 days. FIG. 3B: Freshly isolated human T cells cultured in IL-7 and IL-15 and transduced with lentiviral vector encoding iRFP for the indicated time interval. iRFP+ cells were quantified by flow cytometry. FIG. 3C: Similar results were obtained in an independent experiment from six different donors. FIG. 3D: Representative flow cytometric analysis of non-activated T cells transduced as in FIG. 3A. Naïve, central memory (Tcm), effector memory (Tem), and total effector (Tte) T cell subsets were identified following gating on live singlets, CD3+, CD4+ or CD8+ T cells using CD45RO and CCR7 expression. FIG. 3E: Similar results were obtained in an independent experiment from six different donors. Paired, one-way ANOVA was used, *P<0.05.

FIGS. 4A-4C: CAR lentivirus mediate pseudo-transduction in non-activated T cells. FIG. 4A: Freshly isolated human T cells were cultured in IL-7 and IL-15 and transduced with lentiviral vector encoding a CD19-specific CAR. Gene transduction efficiency was measured after immunostaining with an anti-idiotype antibody for the indicated time interval. Representative flow cytometry plots of CAR expression are shown. FIG. 4B: Non-activated T cells or T cells previously stimulated with anti-CD3/CD28 microbeads were transduced with CAR lentivirus and cocultured with an integrase inhibitor and a RT inhibitor for 4 days. CAR+ cells were quantified by flow cytometry. FIG. 4C: Non-activated T cells were transduced with iRFP lentivirus and cocultured with an integrase inhibitor and a RT inhibitor as in FIG. 4B. iRFP+ cells were quantified by flow cytometry.

FIGS. 5A-5F: Non-activated T cells expressing a CD19-specific CAR control leukemia in xenograft models of ALL. FIG. 5A: Schematic of generation of non-activated CART19 cells in less than 24 hours. FIG. 5B: Schematic of the xenograft model with CART19 cell treatment in NSG mice. FIG. 5C: Total bioluminescence flux in mice treated with 3×10⁶ non-activated T cells transduced as in FIG. 5A (dl), 3×10⁶ CAR+ T cells stimulated with anti-CD3/CD28 microbeads and expanded over 9 days (d9), and 3×10⁶ non-transduced (NTD) control non-activated T cells (n=10 per group). FIGS. 5D-5E: Absolute peripheral blood CD45+ T cell counts in blood collected from mice shown in FIG. 5C at the indicated time following T cell transfer measured by a TruCount assay. The mean of each group is indicated by the solid black line. Groups were compared using the two-tailed, unpaired Mann-Whitney test. *P<0.05, **P<0.01 and ***P<0.001. FIG. 5F: Overall survival of mice by group. P<0.0001 for dl vs NTD and d9 vs NTD by log-rank test. Non-activated CART19 (dl), CAR+ T cells stimulated with anti-CD3/CD28 microbeads (d9), non-transduced, non-activated T cells (NTD).

FIGS. 6A-6E: Transducing conditions can enhance transduction efficiency in non-activated T cells. FIG. 6A: Freshly isolated human T cells were either serum starved by washing and resuspending in serum-free medium or maintained in complete medium for 3 hours and then transduced with a lentiviral vector encoding iRFP for 24 hours in the presence of IL-7 and IL-15 in complete medium. Cells were then maintained in culture for 5 days in IL-7 and IL-15-containing medium prior to determining the iRFP+cell frequency by flow cytometry. Each dot represents transduction frequency determined by flow cytometry from an independent experiment using a different donor. FIG. 6B: Relative fold change of transduction of iRFP+ cells transduced in the presence of 50 μM dNs normalized to iRFP+ cells transduced in complete media without dNs. Data are shown as mean±SD of six experiments performed with different donors. FIG. 6C: Freshly isolated human T cells were transduced with lentiviral vector iRFP cultured in either one well, or two wells, or four wells or eight wells with total culture volume held constant. Cells were then maintained in culture for 5 days in IL-7 and IL-15-containing medium prior to determining the iRFP+cell frequency by flow cytometry. Results are representative of three independent experiments using three different donors. Unpaired Mann-Whitney test, two-tailed was used. *P<0.05, ***P<0.001. FIG. 6D: Schematic of generation of non-activated CART19 cells in 24 hours. (E) CAR+cell frequency as estimated by quantitative PCR analysis of vector copy number in peripheral blood collected at 3 weeks following adoptive transfer of T cells generated as described in panel a. The results are expressed as a percentage of human cells by normalization to the CDKN1A gene, which has two copies in the human diploid genome.

FIGS. 7A-7H: Non-activated T cells expressing a CD19-specific CAR induce potent and durable remission of ALL at low doses. FIG. 7A: Schematic of the xenograft model and CART19 cell treatment in NSG mice. FIGS. 7B-7D: serial quantification of disease burden by bioluminescence imaging (BLI). FIG. 7B: Total bioluminescence flux in mice treated with non-transduced (NTD) control non-activated T cells (n=8). FIG. 7C: Total bioluminescence flux in mice treated with a high (2×10⁶), medium (0.7×10⁶) or low (0.2×10⁶) dose of non-activated T cells transduced as in FIG. 5D. FIG. 7D: Total bioluminescence flux in mice treated with 3×10⁶ CAR+ T cells stimulated with anti-CD3/CD28 microbeads and expanded over 9 days. FIG. 7E: Time to initial anti-leukemic response (i.e. first reduction in bioluminescence) after infusion of non-activated CART19 in relationship to T cell dose. FIG. 7F: Absolute peripheral blood CD45+ T cell counts in blood collected from mice shown in FIGS. 7D-7F at 10 days following T cell transfer measured by a TruCount assay. FIG. 7G: Vector copy number in peripheral blood collected at day 10 following T cell transfer measured by qPCR and normalized to DNA concentration. FIG. 7H: Absolute peripheral blood CD45+ T cell counts in blood collected from mice shown in FIGS. 7B-7D on day 30 following T cell transfer measured by a TruCount assay. The mean of each group is indicated by the solid black line. Groups were compared using the two-tailed, unpaired Mann-Whitney test. *P<0.05, **P<0.01 and ***P<0.001.

FIG. 8: Flow cytometry plots showing transduction efficiency of non-activated T cells maintained in IL-7 and IL-15.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present invention encompass polypeptides and nucleic acids having the sequences specified, or sequences substantially identical or similar thereto, e.g., sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “quiescent T cells” refers to non-proliferating, non-dividing, or resting T cells, e.g., cells in the G0 phase of the cell cycle. T cells may naturally be in a quiescent state. T cell quiescence may be artificially induced using methods or agents generally known in the art, e.g., by serum starvation. In some embodiments, a population of quiescent T cells comprises T cells which have been synchronized in the G0 phase of the cell cycle, e.g., by serum starvation. In some embodiments, quiescent T cells are T cells that are not activated, e.g., not activated via T cell receptor (TCR) or co-receptors (e.g., CD3 and/or CD28). In some embodiments, quiescent T cells are engineered to express a CAR in the absence of T cell activation, e.g., without exposure to any stimulus of T cell activation, e.g., without ex vivo exposure to any stimulus of T cell activation.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In some embodiments, the CAR comprises a chimeric fusion protein comprising an extracellular antigen recognition domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen recognition domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen recognition domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a human or humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises an scFv.

As used herein, the term “binding domain” or “antibody molecule” (also referred to herein as “anti-target binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “anti-tumor effect” and “anti-cancer effect” are used interchangeably and refer to a biological effect which can be manifested by various means, including but not limited to, e.g., a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, or amelioration of various physiological symptoms associated with the cancerous condition. An “anti-tumor effect” or “anti-cancer effect” can also be manifested by the ability of the peptides, polynucleotides, cells and antibodies of the invention in prevention of the occurrence of tumor or cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species. The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” in the context of stimulation by a stimulatory and/or costimulatory molecule refers to a response, e.g., a primary or secondary response, induced by binding of a stimulatory molecule (e.g., a TCR/CD3 complex) and/or a costimulatory molecule (e.g., CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FccRI and CD66d, DAP10 and DAP12. In a specific CAR of the invention, the intracellular signaling domain in any one or more CARS of the invention comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHCs) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FccRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In some embodiments, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In some embodiments, the “zeta stimulatory domain” or a “CD3-zeta stimulatory domain” is the sequence provided as SEQ ID NO: 9 or 10, or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In some embodiments, the “4-1BB costimulatory domain” is the sequence provided as SEQ ID NO: 7 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and costimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen,” “tumor antigen,” “hyperproliferative disorder antigen,” and “antigen associated with a hyperproliferative disorder” interchangeably refer to antigens that are common to specific hyperproliferative disorders. In some embodiments, these terms refer to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some embodiments, the CARs of the present invention include CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser)n, where n is a positive integer equal to or greater than 1. For example, n=1, n=2, n=3. N=4, n=5 and n=6, n=7, n=8, n=9 and n=10 (SEQ ID NO: 42). In some embodiments, the flexible polypeptide linkers include, but are not limited to, (Gly4 Ser)4 (SEQ ID NO: 27) or (Gly4 Ser)3 (SEQ ID NO: 28). In another embodiment, the linkers include multiple repeats of (Gly2Ser), (GlySer) or (Gly3Ser) (SEQ ID NO: 29). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from Rnases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400 (SEQ ID NO: 30). Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In some embodiments the RCARX cell is a T cell, and is referred to as a RCART cell. In some embodiments the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expres sing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In some embodiments, the low, immune enhancing, dose of mTOR inhibitor results in an increase in the number of naive T cells. In some embodiments, the low, immune enhancing, dose of mTOR inhibitor results in one or more of the following:

an increase in the expression of one or more of the following markers: CD62L^{high}, CD127^{high}, CD27±, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62L^{high}, increased CD127^{high}, increased CD27±, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in some embodiments, a response (e.g., complete response or partial response) can involve the absence of detectable MRD (minimal residual disease). In some embodiments, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art and are described more fully below.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, e.g., a selection step, e.g., a negative selection, is performed. The depletion can be a complete or partial depletion of the cell, protein, or macromolecule. In some embodiments, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

Without wishing to be bound by theory, a cell having a “central memory T cell (Tcm) phenotype” expresses CCR7 and CD45RO. In one embodiment, a cell having a central memory T cell phenotype expresses CCR7 and CD45RO, and/or does not express or expresses lower levels of CD45RA as compared to a naive T cell. In one embodiment, a cell having a central memory T cell phenotype expresses CD45RO and CD62L, and/or does not express or expresses lower levels of CD45RA, as compared to a naive T cell. In one embodiment, a cell having a central memory T cell phenotype expresses CCR7, CD45RO, and CD62L, and/or does not express or expresses lower levels of CD45RA as compared to a naive T cell.

Without wishing to be bound by theory, a cell having an “effector memory T cell (Tem) phenotype” does not express or expresses lower levels of CCR7, and expresses higher levels of CD45RO, as compared to a naïve T cell.

Various aspects of the compositions and methods herein are described in further detail below. Additional definitions are set out throughout the specification.

### DESCRIPTION

Provided herein are methods of manufacturing immune effector cells (e.g., T cells or NK cells) that can be engineered with a CAR, e.g., a CAR described herein, reaction mixtures and compositions comprising such cells, and methods of using such cells for treating a disease, such as cancer, in a subject. The traditional CART manufacturing methods involve ex vivo stimulation and expansion, which may induce T cell differentiation, which in turn may reduce T cell engraftment following adoptive transfer. Without wishing to be bound by theory, the methods provided herein eliminate ex vivo activation (e.g., activation using anti-CD3 and/or anti-CD28 antibodies) and expansion and therefore prevent activation-induced differentiation of T cells by enhancing the transduction efficiency of quiescent T cells. In some embodiments, the methods provided herein maintain the original T cell subset phenotype in the input materials and prevent ex vivo differentiation of T cells in the immune effector cells during the manufacturing process. In some embodiments, the methods provided herein do not increase the percentage of terminal differentiated T cells (e.g., T cell subsets distinguished by low levels of CCR7 expression, e.g., as compared to CCR7 expression on naïve T cells or central memory T cells, referred to herein as “CCR7^{low} T cells”) in the immune effector cells during the manufacturing process. In some embodiments, the methods provided herein do not reduce the percentage of naïve T cells in the immune effector cells during the manufacturing process. Using the methods provided herein, freshly isolated quiescent T cells can be transduced with a lentiviral vector to express a CAR without ex vivo activation or expansion. In some embodiments, T cells are incubated in serum-free medium for, e.g., 1, 2, 3, 4, 5, 6, 7, 8, 9, or 10 hours prior to lentiviral transduction. In some embodiments, T cells are transduced with a lentiviral vector to express a CAR in the presence of about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides. In some embodiments, the methods disclosed herein do not involve expanding T cells ex vivo for, e.g., 4, 5, 6, 7, 8, 9, 10, 11, or 12 days. In some embodiments, the methods disclosed herein may manufacture immune effector cells, e.g., T cells, engineered to express a CAR in less than 24, 26, 28, 30, 32, 34, 36, 38, or 40 hours.

### Serum Starvation

Viral attachment and entry represent an important initial phase of transduction during which the RNA genome of a lentiviral vector is inserted into a cell's cytoplasm. Vesicular stomatitis virus g-glycoprotein (VSV-g) pseudotyped lentiviral vectors may bind to low-density lipoprotein receptor (LDL-R) and then internalize by endocytosis. The low transduction efficiency of freshly isolated, human quiescent T cells may be attributed to their low expression of LDL-R. Without wishing to be bound by theory, cholesterol restriction, e.g., serum starvation, e.g., brief serum starvation, prior to lentiviral vector transduction may increase LDL-R expression and endocytosis, thereby enhancing lentiviral vector transduction of quiescent T cells.

In some embodiments, the present disclosure provides methods of making a population of immune cells, e.g., T cells, that express a CAR comprising: (i) incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; and (ii) transducing the population of immune cells with a nucleic acid molecule, e.g., a nucleic acid molecule on a lentiviral vector, encoding the CAR, in a medium comprising serum, e.g., at least about 4, 5, or 6% serum, thereby expressing the CAR, e.g., for about 14-30 hours, e.g., for about 14, 16, 18, 20, 22, 24, 26, or 28 hours. In some embodiments, step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (ii) is performed at a cell concentration of about 1×10⁷ cells/mL.

In some embodiments, the methods further comprise (iii) harvesting the population of immune cells for storage (e.g., reformulating the population of immune cells in cryopreservation media) or administration. In some embodiments, step (iii) is performed no later than 48 hours, e.g., no later than 14, 16, 18, 20, 22, 24, 26, 28, 30, or 32 hours after the beginning of step (i). In some embodiments, step (iii) is performed no later than 14 hours after the beginning of step (i). In some embodiments, step (iii) is performed no later than 16 hours after the beginning of step (i). In some embodiments, step (iii) is performed no later than 18 hours after the beginning of step (i). In some embodiments, step (iii) is performed no later than 20 hours after the beginning of step (i). In some embodiments, step (iii) is performed no later than 22 hours after the beginning of step (i). In some embodiments, step (iii) is performed no later than 24 hours after the beginning of step (i). In some embodiments, step (iii) is performed no later than 26 hours after the beginning of step (i). In some embodiments, step (iii) is performed no later than 28 hours after the beginning of step (i). In some embodiments, step (iii) is performed no later than 30 hours after the beginning of step (i). In some embodiments, step (iii) is performed no later than 32 hours after the beginning of step (i).

In some embodiments, the population of immune cells from step (iii) is not expanded, or is expanded by no more than 10, 20, 30, 40, or 50%, e.g., no more than 10%, compared with the population of immune cells at the beginning of step (i). In some embodiments, the population of immune cells from step (iii) is not expanded compared with the population of immune cells at the beginning of step (i). In some embodiments, the population of immune cells from step (iii) by no more than 5% compared with the population of immune cells at the beginning of step (i). In some embodiments, the population of immune cells from step (iii) by no more than 10% compared with the population of immune cells at the beginning of step (i). In some embodiments, the population of immune cells from step (iii) by no more than 15% compared with the population of immune cells at the beginning of step (i). In some embodiments, the population of immune cells from step (iii) by no more than 20% compared with the population of immune cells at the beginning of step (i). In some embodiments, the population of immune cells from step (iii) by no more than 25% compared with the population of immune cells at the beginning of step (i). In some embodiments, the population of immune cells from step (iii) by no more than 30% compared with the population of immune cells at the beginning of step (i). In some embodiments, the population of immune cells from step (iii) by no more than 35% compared with the population of immune cells at the beginning of step (i). In some embodiments, the population of immune cells from step (iii) by no more than 40% compared with the population of immune cells at the beginning of step (i). In some embodiments, the population of immune cells from step (iii) by no more than 45% compared with the population of immune cells at the beginning of step (i). In some embodiments, the population of immune cells from step (iii) by no more than 50% compared with the population of immune cells at the beginning of step (i). In some embodiments, the population of immune cells from step (iii) by no more than 60% compared with the population of immune cells at the beginning of step (i). In some embodiments, the population of immune cells from step (iii) by no more than 80% compared with the population of immune cells at the beginning of step (i). In some embodiments, the population of immune cells from step (iii) by no more than 100% compared with the population of immune cells at the beginning of step (i). In some embodiments, the population of immune cells from step (iii) by no more than 1.5, 2, 2.5, 3, 3.5, or 4-fold compared with the population of immune cells at the beginning of step (i). In some embodiments, the population of immune cells from step (iii) by no more than 100% compared with the population of immune cells at the beginning of step (i).

In some embodiments, the percentage of naïve cells, e.g., naïve T cells, in the population of immune cells from step (iii) is not reduced, or is reduced by no more than 10, 15, 20, 25, 30, 35, 40, 45, or 50%, compared with the percentage of naïve cells, e.g., naïve T cells, in the population of immune cells at the beginning of step (i). In some embodiments, the percentage of differentiated cells, e.g., differentiated T cells, e.g., terminally differentiated T cells, e.g., CCR7^{low} T cells, in the population of immune cells from step (iii) is not increased, or is increased by no more than 10, 15, 20, 25, 30, 35, 40, 45, or 50%, compared with the percentage of differentiated cells, e.g., differentiated T cells, e.g., terminally differentiated T cells, e.g., CCR7^{low} T cells, in the population of immune cells at the beginning of step (i).

In some embodiments, step (i) comprises incubating the population of immune cells (e.g., T cells, e.g., freshly isolated T cells, e.g., freshly isolated quiescent T cells) in a medium that does not comprise serum for at least about 2-6 hours. In some embodiments, step (i) comprises incubating the population of immune cells (e.g., T cells, e.g., freshly isolated T cells, e.g., freshly isolated quiescent T cells) in a medium that does not comprise serum for about 2 hours. In some embodiments, step (i) comprises incubating the population of immune cells (e.g., T cells, e.g., freshly isolated T cells, e.g., freshly isolated quiescent T cells) in a medium that does not comprise serum for about 3 hours. In some embodiments, step (i) comprises incubating the population of immune cells (e.g., T cells, e.g., freshly isolated T cells, e.g., freshly isolated quiescent T cells) in a medium that does not comprise serum for about 4 hours. In some embodiments, step (i) comprises incubating the population of immune cells (e.g., T cells, e.g., freshly isolated T cells, e.g., freshly isolated quiescent T cells) in a medium that does not comprise serum for about 5 hours. In some embodiments, step (i) comprises incubating the population of immune cells (e.g., T cells, e.g., freshly isolated T cells, e.g., freshly isolated quiescent T cells) in a medium that does not comprise serum for about 6 hours. In some embodiments, step (i) comprises incubating the population of immune cells (e.g., T cells, e.g., freshly isolated T cells, e.g., freshly isolated quiescent T cells) in a medium that does not comprise serum for about 7 hours.

In some embodiments, step (i) increases expression of low-density lipoprotein receptor (LDL-R) in the population of immune cells, e.g., increases expression of LDL-R by at least about 20, 30, 40, 50, 60, 70, 80, 90, 100, 200, 300, 400, 500, 600, 700, 800, 900, or 1000%. In some embodiments, step (i) increases expression of a receptor that is involved in endocytosis, e.g., a receptor that is involved in the endocytosis of a lentiviral vector, in the population of immune cells, e.g., by at least about 20, 40, 60, 80, 100, 500, or 1000%. In some embodiments, step (i) increases transduction efficiency of step (ii) by, e.g., at least about 2, 3, 4, 5, 6, 7, 8, 9, 10, 11, or 12-fold, compared with an otherwise similar method without step (i), e.g., as measured by expression of CAR (e.g., the percentage of CAR-expressing cells) in the population of immune cells at the end of step (ii).

In some embodiments, step (ii) comprises transducing the population of immune cells with a nucleic acid molecule, e.g., a nucleic acid molecule on a lentiviral vector, encoding the CAR in a medium comprising serum, e.g., a medium comprising at least about 3, 4, 5, 6, or 7% serum. In some embodiments, step (ii) comprises transducing the population of immune cells with a lentiviral vector comprising a nucleic acid molecule encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 15, 16, 17, 18, 19, 20, 21, 22, 23, 24, 25, 26, 27, 28, 29, or 30 hours. In some embodiments, step (ii) comprises transducing the population of immune cells with a lentiviral vector comprising a nucleic acid molecule encoding the CAR in a medium comprising about 50 μM deoxynucleosides for about 14-24 hours. In some embodiments, transducing the population of immune cells in a medium comprising deoxynucleosides, e.g., at least about 40 μM-1.5 mM deoxynucleosides, increases transduction efficiency of step (ii) by, e.g., at least about 1, 2, 3, 4, or 5-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (ii). In some embodiments, step (ii) is performed in a medium comprising IL-7 (e.g., about 10 ng/mL of IL-7). In some embodiments, step (ii) is performed in a medium comprising IL-15 (e.g., about 10 ng/mL of IL-15).

In some embodiments, the population of immune cells is not contacted in vitro with an agent that stimulates a CD3/TCR complex and/or an agent that stimulates a costimulatory molecule, e.g., anti-CD3 antibody and/or anti-CD28 antibody. In some embodiments, the population of immune cells is not expanded ex vivo, or if expanded ex vivo, the expansion is shorter than 6 hours, 8 hours, 10 hours, 12 hours, 14 hours, 16 hours, 18 hours, 20 hours, 22 hours, 24 hours, 1.5 days, 2 days, or 3 days.

In some embodiments, the population of immune cells (e.g., T cells) is collected from an apheresis sample (e.g., a leukapheresis sample), e.g., freshly isolated apheresis sample (e.g., freshly isolated leukapheresis sample), from a subject. T cells (e.g., CD8+ and/or CD4+ T cells) are purified using negative selection.

In some embodiments, the nucleic acid molecule is a DNA molecule. In some embodiments, the nucleic acid molecule is an RNA molecule. In some embodiments, the nucleic acid molecule is on a viral vector, e.g., a viral vector chosen from a lentivirus vector, an adenoviral vector, or a retrovirus vector. In some embodiments, the nucleic acid molecule is on a non-viral vector. In some embodiments, the nucleic acid molecule is on a plasmid. In some embodiments, the nucleic acid molecule is not on any vector. In some embodiments, step (ii) comprises transducing the population of immune cells (e.g., T cells) with a lentiviral vector comprising a nucleic acid molecule encoding the CAR.

### Adding Deoxynucleosides in Cell Culture Medium

Resting T cells contain low levels of nucleotides as they are metabolically inactive. Thus, a limited pool of nucleotides exists to support reverse transcription. Without wishing to be bound by theory, supplementing culture medium with deoxynucleosides (dNs) enhances completion of reverse transcription.

In some embodiments, provided herein are methods of making a population of immune cells (e.g., T cells) that express a chimeric antigen receptor (CAR), the method comprising: (1) transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides (e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides) and, e.g., serum (e.g., at least about 4, 5, or 6% serum), e.g., for about 14-30 hours, e.g., for about 14, 16, 18, 20, 22, 24, 26, or 28 hours, thereby expressing the CAR. In some embodiments, step (1) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (1) is performed at a cell concentration of about 1×10⁷ cells/mL.

In some embodiments, the method further comprises (2) harvesting the population of immune cells for storage (e.g., reformulating the population of immune cells in cryopreservation media) or administration. In some embodiments, step (2) is performed no later than 30 hours, e.g., no later than 12, 14, 16, 18, 20, 22, 24, 26, or 28 hours after the beginning of step (1). In some embodiments, step (2) is performed no later than 12 hours after the beginning of step (1). In some embodiments, step (2) is performed no later than 14 hours after the beginning of step (1). In some embodiments, step (2) is performed no later than 16 hours after the beginning of step (1). In some embodiments, step (2) is performed no later than 18 hours after the beginning of step (1). In some embodiments, step (2) is performed no later than 20 hours after the beginning of step (1). In some embodiments, step (2) is performed no later than 22 hours after the beginning of step (1). In some embodiments, step (2) is performed no later than 24 hours after the beginning of step (1). In some embodiments, step (2) is performed no later than 26 hours after the beginning of step (1). In some embodiments, step (2) is performed no later than 28 hours after the beginning of step (1). In some embodiments, step (2) is performed no later than 30 hours after the beginning of step (1).

In some embodiments, the population of immune cells from step (2) is not expanded, or is expanded by no more than 10, 20, 30, 40, or 50%, e.g., no more than 10%, compared with the population of immune cells at the beginning of step (1). In some embodiments, the population of immune cells from step (2) is not expanded. In some embodiments, the population of immune cells from step (2) is expanded by no more than 10, 20, 30, 40, or 50%, e.g., no more than 10%, compared with the population of immune cells at the beginning of step (1). In some embodiments, the population of immune cells from step (2) is expanded by no more than 10% compared with the population of immune cells at the beginning of step (1). In some embodiments, the population of immune cells from step (2) is expanded by no more than 20% compared with the population of immune cells at the beginning of step (1). In some embodiments, the population of immune cells from step (2) is expanded by no more than 30% compared with the population of immune cells at the beginning of step (1). In some embodiments, the population of immune cells from step (2) is expanded by no more than 40% compared with the population of immune cells at the beginning of step (1). In some embodiments, the population of immune cells from step (2) is expanded by no more than 60% compared with the population of immune cells at the beginning of step (1). In some embodiments, the population of immune cells from step (2) is expanded by no more than 80% compared with the population of immune cells at the beginning of step (1). In some embodiments, the population of immune cells from step (2) is expanded by no more than 100% compared with the population of immune cells at the beginning of step (1). In some embodiments, the population of immune cells from step (2) is expanded by no more than 1.5, 2, 2.5, 3, 3.5, or 4-fold compared with the population of immune cells at the beginning of step (1).

In some embodiments, the percentage of naïve cells, e.g., naïve T cells, in the population of immune cells from step (2) is not reduced, or is reduced by no more than 5, 10, 15, 20, 25, 30, 35, 40, 45, or 50%, compared with the percentage of naïve cells, e.g., naïve T cells, in the population of immune cells at the beginning of step (1). In some embodiments, the percentage of differentiated cells, e.g., differentiated T cells, e.g., terminally differentiated T cells, e.g., CCR7^{low} T cells, in the population of immune cells from step (2) is not increased, or is increased by no more than 5, 10, 15, 20, 25, 30, 35, 40, 45, or 50%, compared with the percentage of differentiated cells, e.g., differentiated T cells, e.g., terminally differentiated T cells, e.g., CCR7^{low} T cells, in the population of immune cells at the beginning of step (1).

In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 40 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 50 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 60 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 70 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 80 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 90 μM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 1 mM deoxynucleosides. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 14 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 15 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 16 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 17 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 18 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 19 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 20 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 21 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 22 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 23 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 24 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 25 hours. In some embodiments, step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising deoxynucleosides for about 26 hours.

In some embodiments, transducing the population of immune cells in a medium comprising deoxynucleosides, e.g., at least about 40 μM-1.5 mM deoxynucleosides, increases transduction efficiency of step (1) by, e.g., at least about 1, 2, 3, 4, 5, 6, 7, 8, 9, or 10-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (1).

In some embodiments, step (1) is performed in a medium comprising IL-7 (e.g., about 10 ng/mL of IL-7). In some embodiments, step (1) is performed in a medium comprising IL-15 (e.g., about 10 ng/mL of IL-15).

In some embodiments, prior to step (1), the population of immune cells (e.g., T cells) is incubated in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours. In some embodiments, prior to step (1), the population of immune cells (e.g., T cells) is incubated in a medium that does not comprise serum. In some embodiments, prior to step (1), the population of immune cells (e.g., T cells) is incubated in a medium that does not comprise serum for at least about 2-6 hours. In some embodiments, step (1) is conducted in a medium comprising serum, e.g., a medium comprising at least about 4, 4.5, 5, 5.5, or 6% serum.

In some embodiments, the population of immune cells is not contacted in vitro with an agent that stimulates a CD3/TCR complex and/or an agent that stimulates a costimulatory molecule, e.g., anti-CD3 antibody and/or anti-CD28 antibody.

In some embodiments, the population of immune cells (e.g., T cells) is collected from an apheresis sample (e.g., a leukapheresis sample), e.g., freshly isolated apheresis sample (e.g., freshly isolated leukapheresis sample), from a subject. T cells (e.g., CD8+ and/or CD4+ T cells) are purified using negative selection.

In some embodiments, the nucleic acid molecule is a DNA molecule. In some embodiments, the nucleic acid molecule is an RNA molecule. In some embodiments, the nucleic acid molecule is on a viral vector, e.g., a viral vector chosen from a lentivirus vector, an adenoviral vector, or a retrovirus vector. In some embodiments, the nucleic acid molecule is on a non-viral vector. In some embodiments, the nucleic acid molecule is on a plasmid. In some embodiments, the nucleic acid molecule is not on any vector. In some embodiments, step (ii) comprises transducing the population of immune cells (e.g., T cells) with a lentiviral vector comprising a nucleic acid molecule encoding the CAR.

### Population of CAR-Expressing Cells Manufactured by the Processes Disclosed Herein

In another aspect, the disclosure features an immune effector cell (e.g., T cell or NK cell), e.g., made by any of the manufacturing methods described herein, engineered to express a CAR. In some embodiments, the CAR comprises an antigen binding domain, a transmembrane domain, and an intracellular signaling domain. An exemplary antigen is a cancer associated antigen described herein. In some embodiments, the cell (e.g., T cell or NK cell) is transformed with the CAR and the CAR is expressed on the cell surface. In some embodiments, the cell (e.g., T cell or NK cell) is transduced with a viral vector encoding the CAR. In some embodiments, the viral vector is a retroviral vector. In some embodiments, the viral vector is a lentiviral vector. In some such embodiments, the cell may stably express the CAR. In another embodiment, the cell (e.g., T cell or NK cell) is transfected with a nucleic acid, e.g., mRNA, cDNA, or DNA, encoding a CAR. In some such embodiments, the cell may transiently express the CAR.

In some embodiments, provided herein is a population of cells (e.g., immune effector cells, e.g., T cells or NK cells) made by any of the manufacturing processes described herein (e.g., methods involving serum starvation, or addition of deoxynucleosides described herein), engineered to express a CAR.

In some embodiments, the percentage of naïve cells, e.g., naïve T cells, in the population of immune cells at the end of the manufacturing process is not reduced, or is reduced by no more than 5, 10, 15, 20, 30, 40, or 50%, compared with the percentage of naïve cells, e.g., naïve T cells, in the population of immune cells at the beginning of the manufacturing process. In some embodiments, the percentage of differentiated cells, e.g., differentiated T cells, e.g., terminally differentiated T cells, e.g., CCR7^{low} T cells, in the population of immune cells at the end of the manufacturing process is not increased, or is increased by no more than 5, 10, 15, 20, 30, 40, or 50%, compared with the percentage of differentiated cells, e.g., differentiated T cells, e.g., terminally differentiated T cells, e.g., CCR7^{low} T cells, in the population of immune cells at the beginning of the manufacturing process.

In some embodiments, the population of immune cells at the end of the manufacturing process, after being administered in vivo, persists longer (e.g., at least 10%, 20%, 40%, 60%, 80%, 100%, 2-fold, 4-fold, 6-fold, 8-fold, or 10-fold longer) compared with cells made by an otherwise similar method in which cells are expanded in vitro for at least 6, 7, 8, 9, 10, 11, or 12 days before harvesting. In some embodiments, the population of immune cells at the end of the manufacturing process, after being administered in vivo, expands at a higher level (e.g., at least 10%, 20%, 40%, 60%, 80%, 100%, 2-fold, 4-fold, 6-fold, 8-fold, or 10-fold higher) compared with cells made by an otherwise similar method in which cells are expanded in vitro for at least 6, 7, 8, 9, 10, 11, or 12 days before harvesting. In some embodiments, the population of immune cells at the end of the manufacturing process, after being administered in vivo, exhibits stronger anti-tumor activity, e.g., exhibits anti-tumor activity for a longer period, compared with cells made by an otherwise similar method in which cells are expanded in vitro for at least 6, 7, 8, 9, 10, 11, or 12 days before harvesting.

### Pharmaceutical Composition

Furthermore, the present disclosure provides CAR-expressing cell compositions and their use in medicaments or methods for treating, among other diseases, cancer or any malignancy or autoimmune diseases involving cells or tissues which express an antigen as described herein. In some embodiments, provided herein are pharmaceutical compositions comprising a CAR-expressing cell, e.g., a plurality of CAR-expressing cells, made by a manufacturing process described herein, in combination with one or more pharmaceutically or physiologically acceptable carriers, diluents or excipients.

### Chimeric Antigen Receptor (CAR)

The present invention provides immune effector cells (e.g., T cells or NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that themselves are intracellular, however, fragments (peptides) of such antigens are presented on the surface of the cancer cells by MHC (major histocompatibility complex).

Accordingly, an immune effector cell, e.g., obtained by a method described herein, can be engineered to contain a CAR that targets one of the following cancer associated antigens (tumor antigens): CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ ID | | |
|---|---|---|
| NO | Description | Sequence |
| SEQ ID | EF-1α | CGTGAGGCTCCGGTGCCCGT |
| NO: 11 | promoter (na) | CAGTGGGCAGAGCGCACATC |
| | | GCCCACAGTCCCCGAGAAGT |
| | | TGGGGGGAGGGGTCGGCAAT |
| | | TGAACCGGTGCCTAGAGAAG |
| | | GTGGCGCGGGGTAAACTGGG |
| | | AAAGTGATGTCGTGTACTGG |
| | | CTCCGCCTTTTTCCCGAGGG |
| | | TGGGGGAGAACCGTATATAA |
| | | GTGCAGTAGTCGCCGTGAAC |
| | | GTTCTTTTTCGCAACGGGTT |
| | | TGCCGCCAGAACACAGGTAA |
| | | GTGCCGTGTGTGGTTCCCGC |
| | | GGGCCTGGCCTCTTTACGGG |
| | | TTATGGCCCTTGCGTGCCTT |
| | | GAATTACTTCCACCTGGCTG |
| | | CAGTACGTGATTCTTGATCC |
| | | CGAGCTTCGGGTTGGAAGTG |
| | | GGTGGGAGAGTTCGAGGCCT |
| | | TGCGCTTAAGGAGCCCCTTC |
| | | GCCTCGTGCTTGAGTTGAGG |
| | | CCTGGCCTGGGCGCTGGGGC |
| | | CGCCGCGTGCGAATCTGGTG |
| | | GCACCTTCGCGCCTGTCTCG |
| | | CTGCTTTCGATAAGTCTCTA |
| | | GCCATTTAAAATTTTTGATG |
| | | ACCTGCTGCGACGCTTTTTT |
| | | TCTGGCAAGATAGTCTTGTA |
| | | AATGCGGGCCAAGATCTGCA |
| | | CACTGGTATTTCGGTTTTTG |
| | | GGGCCGCGGGCGGCGACGGG |
| | | GCCCGTGCGTCCCAGCGCAC |
| | | ATGTTCGGCGAGGCGGGGCC |
| | | TGCGAGCGCGGCCACCGAGA |
| | | ATCGGACGGGGGTAGTCTCA |
| | | AGCTGGCCGGCCTGCTCTGG |
| | | TGCCTGGCCTCGCGCCGCCG |
| | | TGTATCGCCCCGCCCTGGGC |
| | | GGCAAGGCTGGCCCGGTCGG |
| | | CACCAGTTGCGTGAGCGGAA |
| | | AGATGGCCGCTTCCCGGCCC |
| | | TGCTGCAGGGAGCTCAAAAT |
| | | GGAGGACGCGGCGCTCGGGA |
| | | GAGCGGGCGGGTGAGTCACC |
| | | CACACAAAGGAAAAGGGCCT |
| | | TTCCGTCCTCAGCCGTCGCT |
| | | TCATGTGACTCCACGGAGTA |
| | | CCGGGCGCCGTCCAGGCACC |
| | | TCGATTAGTTCTCGAGCTTT |
| | | TGGAGTACGTCGTCTTTAGG |
| | | TTGGGGGGAGGGGTTTTATG |
| | | CGATGGAGTTTCCCCACACT |
| | | GAGTGGGTGGAGACTGAAGT |
| | | TAGGCCAGCTTGGCACTTGA |
| | | TGTAATTCTCCTTGGAATTT |
| | | GCCCTTTTTGAGTTTGGATC |
| | | TTGGTTCATTCTCAAGCCTC |
| | | AGACAGTGGTTCAAAGTTTT |
| | | TTTCTTCCATTTCAGGTGTC |
| | | GTGA |
| SEQ ID | Leader (aa) | MALPVTALLLPLALLLHAARP |
| NO: 1 | | |
| SEQ ID | Leader (na) | ATGGCCCTGCCTGTGACAGC |
| NO: 12 | | CCTGCTGCTGCCTCTGGCTC |
| | | TGCTGCTGCATGCCGCTAGA |
| | | CCC |
| SEQ ID | CD 8 hinge (aa) | TTTPAPRPPTPAPTIASQPL |
| NO: 2 | | SLRPEACRPAAGGAVHTRGL |
| | | DFACD |
| SEQ ID | CD8 hinge (na) | ACCACGACGCCAGCGCCGCGA |
| NO: 13 | | CCACCAACACCGGCGCCCAC |
| | | CATCGCGTCGCAGCCCCTGT |
| | | CCCTGCGCCCAGAGGCGTGC |
| | | CGGCCAGCGGCGGGGGGCGC |
| | | AGTGCACACGAGGGGGCTGG |
| | | ACTTCGCCTGTGAT |
| SEQ ID | Ig4 hinge (aa) | ESKYGPPCPPCPAPEFLGGP |
| NO: 3 | | SVFLFPPKPKDTLMISRTPE |
| | | VTCVVVDVSQEDPEVQFNWY |
| | | VDGVEVHNAKTKPREEQFNS |
| | | TYRVVSVLTVLHQDWLNGKE |
| | | YKCKVSNKGLPSSIEKTISK |
| | | AKGQPREPQVYTLPPSQEEM |
| | | TKNQVSLTCLVKGFYPSDIA |
| | | VEWESNGQPENNYKTTPPVL |
| | | DSDGSFFLYSRLTVDKSRWQ |
| | | EGNVFSCSVMHEALHNHYTQ |
| | | KSLSLSLGKM |
| SEQ ID | Ig4 hinge (na) | GAGAGCAAGTACGGCCCTCC |
| NO: 14 | | CTGCCCCCCTTGCCCTGCC |
| | | CCCGAGTTCCTGGGCGGACC |
| | | CAGCGTGTTCCTGTTCCCCC |
| | | CCAAGCCCAAGGACACCCTG |
| | | ATGATCAGCCGGACCCCCGA |
| | | GGTGACCTGTGTGGTGGTGG |
| | | ACGTGTCCCAGGAGGACCCC |
| | | GAGGTCCAGTTCAACTGGTA |
| | | CGTGGACGGCGTGGAGGTGC |
| | | ACAACGCCAAGACCAAGCCC |
| | | CGGGAGGAGCAGTTCAATAG |
| | | CACCTACCGGGTGGTGTCCG |
| | | TGCTGACCGTGCTGCACCAG |
| | | GACTGGCTGAACGGCAAGGA |
| | | ATACAAGTGTAAGGTGTCCA |
| | | ACAAGGGCCTGCCCAGCAGC |
| | | ATCGAGAAAACCATCAGCAA |
| | | GGCCAAGGGCCAGCCTCGGG |
| | | AGCCCCAGGTGTACACCCTG |
| | | CCCCCTAGCCAAGAGGAGAT |
| | | GACCAAGAACCAGGTGTCCC |
| | | TGACCTGCCTGGTGAAGGGC |
| | | TTCTACCCCAGCGACATCGC |
| | | CGTGGAGTGGGAGAGCAACG |
| | | GCCAGCCCGAGAACAACTAC |
| | | AAGACCACCCCCCCTGTGCT |
| | | GGACAGCGACGGCAGCTTCT |
| | | TCCTGTACAGCCGGCTGACC |
| | | GTGGACAAGAGCCGGTGGCA |
| | | GGAGGGCAACGTCTTTAGCT |
| | | GCTCCGTGATGCACGAGGCC |
| | | CTGCACAACCACTACACCCA |
| | | GAAGAGCCTGAGCCTGTCCC |
| | | TGGGCAAGATG |
| SEQ ID | IgD hinge (aa) | RWPESPKAQASSVPTAQPQA |
| NO: 4 | | EGSLAKATTAPATTRNTGRG |
| | | GEEKKKEKEKEEQEERETKT |
| | | PECPSHTQPLGVYLLTPAVQ |
| | | DLWLRDKATFTCFVVGSDLK |
| | | DAHLTWEVAGKVPTGGVEEG |
| | | LLERHSNGSQSQHSRLTLPR |
| | | SLWNAGTSVTCTLNHPSLPP |
| | | QRLMALREPAAQAPVKLSLN |
| | | LLASSDPPEAASWLLCEVSG |
| | | FSPPNILLMWLEDQREVNTS |
| | | GFAPARPPPQPGSTTFWAWS |
| | | VLRVPAPPSPQPATYTCVVS |
| | | HEDSRTLLNASRSLEVSYVT |
| | | DH |
| SEQ ID | IgD hinge (na) | AGGTGGCCCGAAAGTCCCAA |
| NO: 15 | | GGCCCAGGCATCTAGTGTT |
| | | CCTACTGCACAGCCCCAGGC |
| | | AGAAGGCAGCCTAGCCAAAG |
| | | CTACTACTGCACCTGCCACT |
| | | ACGCGCAATACTGGCCGTGG |
| | | CGGGGAGGAGAAGAAAAAGG |
| | | AGAAAGAGAAAGAAGAACAG |
| | | GAAGAGAGGGAGACCAAGAC |
| | | CCCTGAATGTCCATCCCATA |
| | | CCCAGCCGCTGGGCGTCTAT |
| | | CTCTTGACTCCCGCAGTACA |
| | | GGACTTGTGGCTTAGAGATA |
| | | AGGCCACCTTTACATGTTTC |
| | | GTCGTGGGCTCTGACCTGAA |
| | | GGATGCCCATTTGACTTGGG |
| | | AGGTTGCCGGAAAGGTACCC |
| | | ACAGGGGGGGTTGAGGAAGG |
| | | GTTGCTGGAGCGCCATTCCA |
| | | ATGGCTCTCAGAGCCAGCAC |
| | | TCAAGACTCACCCTTCCGAG |
| | | ATCCCTGTGGAACGCCGGGA |
| | | CCTCTGTCACATGTACTCTA |
| | | AATCATCCTAGCCTGCCCCC |
| | | ACAGCGTCTGATGGCCCTTA |
| | | GAGAGCCAGCCGCCCAGGCA |
| | | CCAGTTAAGCTTAGCCTGAA |
| | | TCTGCTCGCCAGTAGTGATC |
| | | CCCCAGAGGCCGCCAGCTGG |
| | | CTCTTATGCGAAGTGTCCGG |
| | | CTTTAGCCCGCCCAACATCT |
| | | TGCTCATGTGGCTGGAGGAC |
| | | CAGCGAGAAGTGAACACCAG |
| | | CGGCTTCGCTCCAGCCCGGC |
| | | CCCCACCCCAGCCGGGTTCT |
| | | ACCACATTCTGGGCCTGGAG |
| | | TGTCTTAAGGGTCCCAGCAC |
| | | CACCTAGCCCCCAGCCAGCC |
| | | ACATACACCTGTGTTGTGTC |
| | | CCATGAAGATAGCAGGACCC |
| | | TGCTAAATGCTTCTAGGAGT |
| | | CTGGAGGTTTCCTACGTGAC |
| | | TGACCATT |
| SEQ ID | CD8 | IYIWAPLAGTCGVLLLSLVI |
| NO: 6 | Transmembrane | TLYC |
| | (aa) | |
| SEQ ID | CDS | ATCTACATCTGGGCGCCCTT |
| NO: 17 | Transmembrane | GGCCGGGACTTGTGGGGTCC |
| | (na) | TTCTCCTGTCACTGGTTATC |
| | | ACCCTTTACTGC |
| SEQ ID | 4-IBB | KRGRKKLLYIEKQPEMRPVQ |
| NO: 7 | intracellular | TTQEEDGCSCREPEEEEGGC |
| | domain (aa) | EL |
| SEQ ID | 4-IBB | AAACGGGGCAGAAAGAAACT |
| NO: 18 | intracellular | CCTGTATATATTCAAACAAC |
| | domain (na) | CATTTATGAGACCAGTACAA |
| | | ACTACTCAAGAGGAAGATGG |
| | | CTGTAGCTGCCGATTTCCAG |
| | | AAGAAGAAGAAGGAGGATGT |
| | | GAACTG |
| SEQ ID | CD27 (aa) | QRRKYRSNKGESPVEPAEPC |
| NO: 8 | | RYSCPREEEGSTIPIQEDYR |
| | | KPEPACSP |
| SEQ ID | CD27 (na) | AGGAGTAAGAGGAGCAGGCT |
| NO: 19 | | CCTGCACAGTGACTACATGA |
| | | ACATGACTCCCCGCCGCCCC |
| | | GGGCCCACCCGCAAGCATTA |
| | | CCAC.CCCTATGCCCCACCA |
| | | CGCGACTTCGCAGCCTATCG |
| | | CTCC |
| SEQ ID | CD3-zeta (aa) | RVKFSRSADAPAYKQGQNQL |
| NO: 9 | (Q/K mutant) | YNELNLGRREEYDVLDKRRG |
| | | RDPEMGGKPRRKNPQEGLYN |
| | | ELQKDKMAEAYSEIGMKGER |
| | | RRGKGHDGLYQGLSTATKDT |
| | | YDALHMQALPPR |
| SEQ ID | CD3-zeta (na) | AGAGTGAAGTTCAGCAGGAG |
| NO: 20 | (Q/K mutant) | CGCAGACGCCCCCGCGTACA |
| | | AGCAGGGCCAGAACCAGCTC |
| | | TATAACGAGCTCAATCTAGG |
| | | ACGAAGAGAGGAGTACGATG |
| | | TTTTGGACAAGAGACGTGGC |
| | | CGGGACCCTGAGATGGGGGG |
| | | AAAGCCGAGAAGGAAGAACC |
| | | CTCAGGAAGGCCTGTACAAT |
| | | GAACTGCAGAAAGATAAGAT |
| | | GGCGGAGGCCTACAGTGAGA |
| | | TTGGGATGAAAGGCGAGCGC |
| | | CGGAGGGGCAAGGGGCACGA |
| | | TGGCCTTTACCAGGGTCTCA |
| | | GTACAGCCACCAAGGACACC |
| | | TACGACGCCCTTCACATGCA |
| | | GGCCCTGCCCCCTCGC |
| SEQ ID | CD3-zeta (aa) | RVKFSRSADAPAYQQGQNQL |
| NO: 10 | (NCBI | YNELNLGRREEYDVLDKRRG |
| | Reference | RDPEMGGKPRRKNPQEGLYN |
| | Sequence | ELQKDKMAEAYSEIGMKGER |
| | NM_000734.3) | RRGKGHDGLYQGLSTATKDT |
| | | YDALHMQALPPR |
| SEQ ID | CD3-zeta (na) | AGAGTGAAGTTCAGCAGGAG |
| NO: 21 | (NCBI | CGCAGACGCCCCCGCGTACC |
| | Reference | AGCAGGGCCAGAACCAGCTC |
| | Sequence | TATAACGAGCTCAATCTAGG |
| | NM_000734.3) | ACGAAGAGAGGAGTACGATG |
| | | TTTTGGACAAGAGACGTGGC |
| | | CGGGACCCTGAGATGGGGGG |
| | | AAAGCCGAGAAGGAAGAACC |
| | | CTCAGGAAGGCCTGTACAAT |
| | | GAACTGCAGAA |
| | | AGATAAGATGGCGGAGGCCT |
| | | ACAGTGAGATTGGGATGAAA |
| | | GGCGAGCGCCGGAGGGGCAA |
| | | GGGGCACGATGGCCTTTACC |
| | | AGGGTCTCAGTACAGCCACC |
| | | AAGGACACCTACGACGCCCT |
| | | TCACATGCAGGCCCTGCCCC |
| | | CTCGC |
| SEQ ID | CD28 | RSKRSRLLHSDYMNMTPRRP |
| NO: 36 | Intracellular | GPTRKHYQPYAPPRDEAAYR |
| | domain | S |
| | (amino acid | |
| | sequence) | |
| SEQ ID | CD28 | AGGAGTAAGAGGAGCAGGCT |
| NO: 37 | Intracellular | CCTGCACAGTGACTACATGA |
| | domain | ACATGACTCCCCGCCGCCCC |
| | (nucleotide | GGGCCCACCCGCAAGCATTA |
| | sequence) | CCAGCCCTATGCCCCACCAC |
| | | GCGACTTCGCAGCCTATCGC |
| | | TCC |
| SEQ ID | ICOS | T K K K Y S S S V H |
| NO: 38 | Intracellular | D P N G E Y M E M R |
| | domain | A V N T A K K S R L |
| | (amino acid | T D V T L |
| | sequence) | |
| SEQ ID | ICOS | ACAAAAAAGAAGTATTCATC |
| NO: 39 | Intracellular | CAGTGTGCACGACCCTAACG |
| | domain | GTGAATACATGTTCATGAGA |
| | (nucleotide | GCAGTGAACACAGCCAAAAA |
| | sequence) | ATCCAGACTCACAGATGTGA |
| | | CCCTA |
| SEQ ID | GS hinge/ | GGGGSGGGGS |
| NO: 5 | linker | |
| | (aa) | |
| SEQ ID | GS hinge/ | GGTCGCGCAGGTTCTGGAGG |
| NO: 16 | linker | TGGAGGTTCC |
| | (na) | |
| SEQ ID | GS hinge/ | GGTGGCGGAGGTTCTGGAGG |
| NO: 40 | linker | TGGGGGTTCC |
| | (na) | |
| SEQ ID | linker | GGGGS |
| NO: 25 | | |
| SEQ ID | linker | (Gly-Gly-Gly- |
| NO: 26 | | Gly-Ser)n, |
| | | where |
| | | n = 1-6, e.g., |
| | | GGGGSGGGGS |
| | | GGGGSGGGGS |
| | | GGGGSGGGGS |
| SEQ ID | linker | GGGGSGGGGSGGGGSGGGGS |
| NO: 27 | | |
| SEQ ID | linker | GGGGSGGGGSGGGGS |
| NO: 28 | | |
| SEQ ID | linker | GGGS |
| NO: 29 | | |
| SEQ ID | linker | (Gly-Gly-Gly-Ser)n |
| NO: 41 | | where n is a positive |
| | | integer equal to or |
| | | greater than 1 |
| SEQ ID | linker | (Gly-Gly-Gly-Ser)n, |
| NO: 42 | | where n = 1-10. |
| | | e.g., GGGSGGGSGG |
| | | GSGGGSGGGS GGGSGGGSGG |
| | | GSGGGSGGGS |
| SEQ ID | linker | GSTSGSGKPGSGEGSTKG |
| NO: 43 | | |
| SEQ ID | polyA | (A)₅₀₀₀ |
| NO: 30 | | This sequence may |
| | | encompass 50-5000 |
| | | adenines. |
| SEQ ID | polyT | (T)₁₀₀ |
| NO: 31 | | |
| SEQ ID | polyT | (T)₅₀₀₀ |
| NO: 32 | | This sequence may |
| | | encompass 50-5000 |
| | | thymines. |
| SEQ ID | polyA | (A)₅₀₀₀ |
| NO: 33 | | This sequence may |
| | | encompass 100-5000 |
| | | adenines. |
| SEQ ID | polyA | (A)₄₀₀ |
| NO: 34 | | This sequence may |
| | | encompass 100-400 |
| | | adenines. |
| SEQ ID | PolyA | (A)₂₀₀₀ |
| NO: 35 | | This sequence may |
| | | encompass 50-2000 |
| | | adenines. |
| SEQ ID | PD1 CAR (aa) | pgwfldspdrpwnpptfspa |
| NO: 22 | | llvvtegdnatftcsfsnts |
| | | esfvlnwvrmspsnqtdkla |
| | | afpedrsqpgqdcrfrvtql |
| | | pngrdfhmsvvrarrndsgt |
| | | vlcgaislapkaqikeslra |
| | | elrvterraevptahpspsp |
| | | rpagafqtlvtttpaprppt |
| | | paptiasqplslrpeacrpa |
| | | aggavhtrgldfacdiyiwa |
| | | plagtcgvlllslvitlyck |
| | | rgrkkllyifkqpfmrpvqt |
| | | tqeedgcscrfpeeeeggce |
| | | lrvkfsrsadapaykqgqnq |
| | | lynelnlgrreeydvldkrr |
| | | grdpemggkprrknpqegly |
| | | nelqkdkmaeayseigmkge |
| | | rrrgkghdglyqglstatkd |
| | | tydalhmqalppr |
| SEQ ID | PD-1 CAR (na) | atggccctccctgtcactgc |
| NO: 23 | (PD1 ECD | cctgcttctccccctcgcac |
| | underlined) | tcctgctccacgccgctaga |
| | | ccacccggatggtttctgga |
| | | ctctccggatcgcccgtgga |
| | | atcccccaaccttctcaccg |
| | | gcactcttggttgtgactga |
| | | gggcgataatgcgaccttca |
| | | cgtgctcgttctccaacacc |
| | | tccgaatcattcgtgctgaa |
| | | ctggtaccgcatgagcccgt |
| | | caaaccagaccgacaagctc |
| | | gccgcgtttccggaagatcg |
| | | gtcgcaaccgggacaggatt |
| | | gtcggttccgcgtgactcaa |
| | | ctgccgaatggcagagactt |
| | | ccacatgagcgtggtccgcg |
| | | ctaggcgaaacgactccggg |
| | | acctacctgtgcggagccat |
| | | ctcgctggcgcctaaggccc |
| | | aaatcaaagagagcttgagg |
| | | gccgaactgagagtgaccga |
| | | gcgcagagctgaggtgccaa |
| | | ctgcacatccatccccatcg |
| | | cctcggcctgcggggcagtt |
| | | tcagaccctggtcacgacca |
| | | ctccggcgccgcgcccaccg |
| | | actccggccccaactatcgc |
| | | gagccagcccctgtcgctga |
| | | ggccggaagcatgccgccct |
| | | gccgccggaggtgctgtgca |
| | | tacccggggattggacttcg |
| | | catgcgacatctacatttgg |
| | | gctcctctcgccggaacttg |
| | | tggcgtgctccttctgtccc |
| | | tggtcatcaccctgtactgc |
| | | aagcggggtcggaaaaagct |
| | | tctgtacattttcaagcagc |
| | | ccttcatgaggcccgtgcaa |
| | | accacccaggaggaggacgg |
| | | ttgctcctgccggttccccg |
| | | aagaggaagaaggaggttgc |
| | | gagctgcgcgtgaagttctc |
| | | ccggagcgccgacgcccccg |
| | | cctataagcagggccagaac |
| | | cagctgtacaacgaactgaa |
| | | cctgggacggcgggaagagt |
| | | acgatgtgctggacaagcgg |
| | | cgcggccgggaccccgaaat |
| | | gggcgggaagcctagaagaa |
| | | agaaccctcaggaaggcctg |
| | | tataacgagctgcagaagga |
| | | caagatggccgaggcctact |
| | | ccgaaattgggatgaaggga |
| | | gagcggcggaggggaaaggg |
| | | gcacgacggcctgtaccaag |
| | | gactgtccaccgccaccaag |
| | | gacacatacgatgccctgca |
| | | catgcaggcccttccccctc |
| | | gc |
| SEQ ID | PD-1 CAR (aa) | Malpvtalllplalllhaarp |
| NO: 24 | with signal | pgwfldspdrpwnpptfspa |
| | (PD1 ECD | llvvtegdnatftcsfsnts |
| | underlined) | esfvlnwvrmspsnqtdkla |
| | | afpedrsqpgqdcrfrvtql |
| | | pngrdfhmsvvrarmdsgty |
| | | lcgaislapkaqikeslrae |
| | | lrvterracvptahpspspr |
| | | pagafqtlvtttpaprpptp |
| | | aptiasqplslrpeacrpaa |
| | | ggavhtrgldfacdiyiwap |
| | | lagtcgvlllslvitlyckr |
| | | grkkllyifkqpfmrpvqtt |
| | | qeedgcscrfpeeeeggcel |
| | | rvkfsrsadapaykqgqnql |
| | | ynelnlgrreeydvldkrrg |
| | | rdpemggkprrknpqeglyn |
| | | elqkdkmaeayseigmkger |
| | | rrgkghdglyqglstatkdt |
| | | ydalhmqalppr |

Bispecific CARs

In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, e.g., the same protein (or subunit of a multimeric protein). In some embodiments the first and second epitopes overlap. In some embodiments the first and second epitopes do not overlap. In some embodiments the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules, and various configurations for bispecific antibody molecules, are described in, e.g., paragraphs 455-458 of WO2015/142675, filed Mar. 13, 2015, which is incorporated by reference in its entirety.

In some embodiments, the bispecific antibody molecule is characterized by a first immunoglobulin variable domain sequence, e.g., a scFv, which has binding specificity for CD19, e.g., comprises a scFv as described herein, or comprises the light chain CDRs and/or heavy chain CDRs from a scFv described herein, and a second immunoglobulin variable domain sequence that has binding specificity for a second epitope on a different antigen.

Chimeric TCR

In some embodiments, the antibodies and antibody fragments of the present invention (e.g., CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, e.g., by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

Non-Antibody Scaffolds

In embodiments, the antigen binding domain comprises a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

In some embodiments the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen binding domain (e.g., antibody or antibody fragment, TCR or TCR fragment) that binds specifically to a tumor antigen, e.g., a tumor antigen described herein, and an intracellular signaling domain. The intracellular signaling domain can comprise a costimulatory signaling domain and/or a primary signaling domain, e.g., a zeta chain. As described elsewhere, the methods described herein can include transducing a cell, e.g., from the population of T regulatory-depleted cells, with a nucleic acid encoding a CAR, e.g., a CAR described herein.

In specific aspects, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signaling domain that includes SEQ ID NO:7 or SEQ ID NO:16 and a CD3 zeta sequence that includes SEQ ID NO:9 or SEQ ID NO:10, e.g., wherein the domains are contiguous with and in the same reading frame to form a single fusion protein.

In some embodiments, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In some embodiments, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In some embodiments, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, e.g., a retroviral or lentiviral vector construct.

Nucleic acids encoding a CAR can also be introduced into the immune effector cell using, e.g., an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (SEQ ID NO: 35) (e.g., described in the Examples, e.g., SEQ ID NO:35). RNA so produced can efficiently transfect different kinds of cells. In some embodiments, the template includes sequences for the CAR. In some embodiments, an RNA CAR vector is transduced into a cell, e.g., a T cell by electroporation.

### Antigen Binding Domain

In some embodiments, a plurality of the immune effector cells, e.g., the population of T regulatory-depleted cells, include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In some embodiments, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CD19 CAR

In some embodiments, the CAR-expressing cell described herein is a CD19 CAR-expressing cell (e.g., a cell expressing a CAR that binds to human CD19).

In some embodiments, the antigen binding domain of the CD19 CAR has the same or a similar binding specificity as the FMC63 scFv fragment described in Nicholson et al. Mol. Immun. 34 (16-17): 1157-1165 (1997). In some embodiments, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (e.g., a humanized antigen binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000.

In some embodiments, the amino acid sequence is:

diqmtqttsslsaslgdrvtiscrasqdiskylnwyqqkpdgtvklliyhtsrlhsgvpsrfsgsgsgtdysltisnleqediat yfcqqgntlpytfgggtkleitggggsggggsggggsevklqesgpglvapsqslsvtctvsgvslpdygvswirqpprkglewlgv iwgsettyynsalksrltiikdnsksqvflkmnslqtddtaiyycakhyyygsyamdywgqgtsvtvsstttpaprpptpaptiasq plslrpeacrpaaggavhtrgldfacdiyiwaplagtcgvlllslvitlyckrgrkkllyifkqpfmrpvqttqeedgcscrfpeeeeggc elrvkfsrsadapaykqgqnqlynelnlgrreeydvldkrrgrdpemggkprrknpqeglynelqkdkmaeayseigmkgerrrg kghdglyqglstatkdtydalhmqalppr (SEQ ID NO: 123), or a sequence substantially homologous thereto.

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the CAR molecule is a humanized CD19 CAR comprising the amino acid sequence of:

(SEQ ID NO: 124)
EIVMTQSPATLSLSPGERATLSCRASQDISKYLNW
YQQKPGQAPRLLIYHTSRLHSGIPARFSGSGSGTD
YTLTISSLQPEDFAVYFCQQGNTLPYTFGQGTKLE
IKGGGGSGGGGSGGGGSQVQLQESGPGLVKPSETL
SLTCTVSGVSLPDYGVSWIRQPPGKGLEWIGVIWG
SETTYYQSSLKSRVTISKDNSKNQVSLKLSSVTAA
DTAVYYCAKHYYYGGSYAMDYWGQGTLVTVSS

In some embodiments, the CAR molecule is a humanized CD19 CAR comprising the amino acid sequence of:

(SEQ ID NO: 125)
EIVMTQSPATLSLSPGERATLSCRASQDISKYLNW
YQQKPGQAPRLLIYHTSRLHSGIPARFSGSGSGTD
YTLTISSLQPEDFAVYFCQQGNTLPYTFGQGTKLE
IKGGGGSGGGGSGGGGSQVQLQESGPGLVKPSETL
SLTCTVSGVSLPDYGVSWIRQPPGKGLEWIGVIWG
SETTYYQSSLKSRVTISKDNSKNQVSLKLSSVTAA
DTAVYYCAKHYYYGGSYAMDYWGQGTLVTVSSTTT
PAPRPPTPAPTIASQPLSLRPEACRPAAGGAVHTR
GLDFACDIYIWAPLAGTCGVLLLSLVITLYCKRGR
KKLLYIFKQPFMRPVQTTQEEDGCSCRFPEEEEGG
CELRVKFSRSADAPAYKQGQNQLYNELNLGRREEY
DVLDKRRGRDPEMGGKPRRKNPQEGLYNELQKDKM
AEAYSEIGMKGERRRGKGHDGLYQGLSTATKDTYD
ALHMQALPPR

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16^{th} Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### BCMA CAR

In some embodiments, the CAR-expressing cell described herein is a BCMA CAR-expressing cell (e.g., a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In certain embodiments, the domains are contiguous and in the same reading frame to form a single fusion protein. In other embodiments, the domain are in separate polypeptides, e.g., as in an RCAR molecule as described herein.

In some embodiments, the BCMA CAR molecule includes one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

### CD20 CAR

In some embodiments, the CAR-expressing cell described herein is a CD20 CAR-expressing cell (e.g., a cell expressing a CAR that binds to human CD20). In some embodiments, the CD20 CAR-expressing cell includes an antigen binding domain according to WO2016/164731 and PCT/US2017/055627, incorporated herein by reference. Exemplary CD20-binding sequences or CD20 CAR sequences are disclosed in, e.g., Tables 1-5 of PCT/US2017/055627. In some embodiments, the CD20 CAR comprises a CDR, variable region, scFv, or full-length sequence of a CD20 CAR disclosed in PCT/US2017/055627 or WO2016/164731.

### CD22 CAR

In some embodiments, the CAR-expressing cell described herein is a CD22 CAR-expressing cell (e.g., a cell expressing a CAR that binds to human CD22). In some embodiments, the CD22 CAR-expressing cell includes an antigen binding domain according to WO2016/164731 and PCT/US2017/055627, incorporated herein by reference. Exemplary CD22-binding sequences or CD22 CAR sequences are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22 CAR sequences comprise a CDR, variable region, scFv or full-length sequence of a CD22 CAR disclosed in PCT/US2017/055627 or WO2016/164731.

### EGFR CAR

In some embodiments, the CAR-expressing cell described herein is an EGFR CAR-expressing cell (e.g., a cell expressing a CAR that binds to human EGFR). In some embodiments, the CAR-expressing cell described herein is an EGFRvIII CAR-expressing cell (e.g., a cell expressing a CAR that binds to human EGFRvIII). Exemplary EGFRvIII CARs can include sequences disclosed in WO2014/130657, e.g., Table 2 of WO2014/130657, incorporated herein by reference.

Exemplary EGFRvIII-binding sequences or EGFR CAR sequences may comprise a CDR, a variable region, an scFv, or a full-length CAR sequence of a EGFR CAR disclosed in WO2014/130657.

### Mesothelin CAR

In some embodiments, the CAR-expressing cell described herein is a mesothelin CAR-expressing cell (e.g., a cell expressing a CAR that binds to human mesothelin). Exemplary mesothelin CARs can include sequences disclosed in WO2015090230 and WO2017112741, e.g., Tables 2, 3, 4, and 5 of WO2017112741, incorporated herein by reference.

### Other Exemplary CARs

In other embodiments, the CAR-expressing cells can specifically bind to CD123, e.g., can include a CAR molecule (e.g., any of the CAR1 to CARE), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635. In other embodiments, the CAR-expressing cells can specifically bind to CD123, e.g., can include a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In some embodiments, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In other embodiments, the CAR-expressing cells can specifically bind to CLL-1, e.g., can include a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In some embodiments, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference. In other embodiments, the CAR-expressing cells can specifically bind to CD33, e.g., can include a CAR molecule (e.g., any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In some embodiments, the antigen binding domain comprises one, two three (e.g., all three) heavy chain CDRs, HC CDR1, HC CDR2 and HC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (e.g., all three) light chain CDRs, LC CDR1, LC CDR2 and LC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1. In embodiments, the antigen binding domain targets CD19 and is described in US-2015-0283178-A1. In embodiments, the antigen binding domain targets CD123 and is described in US2014/0322212A1, US2016/0068601A1. In embodiments, the antigen binding domain targets CLL1 and is described in US2016/0051651A1. In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to GFR ALPHA-4, e.g., can include a CAR molecule, or an antigen binding domain according to Table 2 of WO2016/025880, incorporated herein by reference. The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In some embodiments, the antigen binding domain of any of the CAR molecules described herein (e.g., any of CD19, CD123, EGFRvIII, CD33, mesothelin, BCMA, and GFR ALPHA-4) comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGicp(1-1)Cer); transglutaminase 5 (TGS5); high molecular weight-melanoma-associated antigen (HMWMAA); o-acetyl-GD2 ganglioside (OacGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member lA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the anti-tumor antigen binding domain is a fragment, e.g., a single chain variable fragment (scFv). In some embodiments, the anti-a cancer associate antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In some embodiments, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser)n, where n is a positive integer equal to or greater than 1 (SEQ ID NO: 126). In some embodiments, the linker can be (Gly4Ser)₄ (SEQ ID NO: 27) or (Gly4Ser)₃(SEQ ID NO: 28). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the CAR-expressing cell, e.g., CART cell surface. In a different aspect the amino acid sequence of the transmembrane domain may be modified or substituted so as to minimize interactions with the binding domains of the native binding partner present in the same CAR-expressing cell, e.g., CART.

The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target. A transmembrane domain of particular use in this invention may include at least the transmembrane region(s) of, e.g., the alpha, beta or zeta chain of T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, e.g., the antigen binding domain of the CAR, via a hinge, e.g., a hinge from a human protein. For example, in some embodiments, the hinge can be a human Ig (immunoglobulin) hinge, e.g., an IgG4 hinge, or a CD8a hinge. In some embodiments, the hinge or spacer comprises (e.g., consists of) the amino acid sequence of SEQ ID NO: 2. In some embodiments, the transmembrane domain comprises (e.g., consists of) a transmembrane domain of SEQ ID NO: 6.

In some embodiments, the hinge or spacer comprises an IgG4 hinge. For example, in some embodiments, the hinge or spacer comprises a hinge of SEQ ID NO: 3. In some embodiments, the hinge or spacer comprises a hinge encoded by the nucleotide sequence of SEQ ID NO: 14.

In some embodiments, the hinge or spacer comprises an IgD hinge. For example, in some embodiments, the hinge or spacer comprises a hinge of the amino acid sequence of SEQ ID NO: 4. In some embodiments, the hinge or spacer comprises a hinge encoded by the nucleotide sequence of SEQ ID NO:15.

In some embodiments, the transmembrane domain may be recombinant, in which case it will comprise predominantly hydrophobic residues such as leucine and valine. In some embodiments a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some embodiments, the linker comprises the amino acid sequence of SEQ ID NO: 5. In some embodiments, the linker is encoded by a nucleotide sequence of SEQ ID NO: 16.

In some embodiments, the hinge or spacer comprises a KIR2DS2 hinge.

### Cytoplasmic Domain

The cytoplasmic domain or region of a CAR of the present invention includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FccRI, DAP10, DAP12, and CD66d. In some embodiments, a CAR of the invention comprises an intracellular signaling domain, e.g., a primary signaling domain of CD3-zeta.

In some embodiments, a primary signaling domain comprises a modified ITAM domain, e.g., a mutated ITAM domain which has altered (e.g., increased or decreased) activity as compared to the native ITAM domain. In some embodiments, a primary signaling domain comprises a modified ITAM-containing primary intracellular signaling domain, e.g., an optimized and/or truncated ITAM-containing primary intracellular signaling domain. In some embodiments, a primary signaling domain comprises one, two, three, four or more ITAM motifs.

Further examples of molecules containing a primary intracellular signaling domain that are of particular use in the invention include those of DAP10, DAP12, and CD32.

The intracellular signaling domain of the CAR can comprise the primary signaling domain, e.g., CD3-zeta signaling domain, by itself or it can be combined with any other desired intracellular signaling domain(s) useful in the context of a CAR of the invention. For example, the intracellular signaling domain of the CAR can comprise a primary signaling domain, e.g., CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In some embodiments, a glycine-serine doublet can be used as a suitable linker. In some embodiments, a single amino acid, e.g., an alanine, a glycine, can be used as a suitable linker.

In some embodiments, the intracellular signaling domain is designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 7. In some embodiments, the signaling domain of CD3-zeta is a signaling domain of SEQ ID NO: 9 (mutant CD3zeta) or SEQ ID NO: 10 (wild type human CD3zeta).

In some embodiments, the intracellular signaling domain is designed to comprise the signaling domain of CD3-zeta and the signaling domain of CD27. In some embodiments, the signaling domain of CD27 comprises the amino acid sequence of SEQ ID NO: 8. In some embodiments, the signaling domain of CD27 is encoded by the nucleic acid sequence of SEQ ID NO: 19.

In some embodiments, the intracellular is designed to comprise the signaling domain of CD3-zeta and the signaling domain of CD28. In some embodiments, the signaling domain of CD28 comprises the amino acid sequence of SEQ ID NO: 36. In some embodiments, the signaling domain of CD28 is encoded by the nucleic acid sequence of SEQ ID NO: 37.

In some embodiments, the intracellular is designed to comprise the signaling domain of CD3-zeta and the signaling domain of ICOS. In some embodiments, the signaling domain of ICOS comprises the amino acid sequence of SEQ ID NO: 38. In some embodiments, the signaling domain of ICOS is encoded by the nucleic acid sequence of SEQ ID NO: 39.

Co-Expression of CAR with Other Molecules or Agents

Co-Expression of a Second CAR

In some embodiments, the CAR-expressing cell described herein can further comprise a second CAR, e.g., a second CAR that includes a different antigen binding domain, e.g., to the same target (e.g., CD19) or a different target (e.g., a target other than CD19, e.g., a target described herein). In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In some embodiments, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In another embodiment, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In some embodiments, the CAR-expressing cell comprises an XCAR described herein and an inhibitory CAR. In some embodiments, the inhibitory CAR comprises an antigen binding domain that binds an antigen found on normal cells but not cancer cells, e.g., normal cells that also express X. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta).

In some embodiments, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In some embodiments, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising antigen binding domains that minimize such interactions, as well as methods of making and using such cells and nucleic acids. In some embodiments the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, e.g., 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, e.g., 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in some embodiments, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In some embodiments the agent is an shRNA, e.g., an shRNA described herein. In some embodiments, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In some embodiments, the agent which inhibits an inhibitory molecule comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In some embodiments, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In some embodiments, the PD1 CAR, when used in combinations with an XCAR described herein, improves the persistence of the T cell. In some embodiments, the CAR is a PD1 CAR comprising the extracellular domain of PD1 indicated as underlined in SEQ ID NO: 24. In some embodiments, the PD1 CAR comprises the amino acid sequence of SEQ ID NO: 24.

In some embodiments, the PD1 CAR comprises the amino acid sequence of SEQ ID NO: 22.

In some embodiments, the agent comprises a nucleic acid sequence encoding the PD1 CAR, e.g., the PD1 CAR described herein. In some embodiments, the nucleic acid sequence for the PD1 CAR is provided as SEQ ID NO: 23, with the PD1 ECD underlined.

In another example, in some embodiments, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In some embodiments, the costimulatory molecule ligand is 4-1BBL. In some embodiments, the costimulatory ligand is CD80 or CD86. In some embodiments, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In some embodiments, the chemokine receptor molecule to be expressed with a CAR described herein is selected based on the chemokine(s) secreted by the tumor. In some embodiments, the CAR-expressing cell described herein further comprises, e.g., expresses, a CCR2b receptor or a CXCR2 receptor. In some embodiments, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, e.g., made by a method described herein, that includes a nucleic acid molecule encoding one or more CAR constructs described herein. In some embodiments, the nucleic acid molecule is provided as a messenger RNA transcript. In some embodiments, the nucleic acid molecule is provided as a DNA construct.

The nucleic acid molecules described herein can be a DNA molecule, an RNA molecule, or a combination thereof. In some embodiments, the nucleic acid molecule is an mRNA encoding a CAR polypeptide as described herein. In other embodiments, the nucleic acid molecule is a vector that includes any of the aforesaid nucleic acid molecules.

In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Accordingly, in some embodiments, an immune effector cell, e.g., made by a method described herein, includes a nucleic acid molecule encoding a chimeric antigen receptor (CAR), wherein the CAR comprises an antigen binding domain that binds to a tumor antigen described herein, a transmembrane domain (e.g., a transmembrane domain described herein), and an intracellular signaling domain (e.g., an intracellular signaling domain described herein) comprising a stimulatory domain, e.g., a costimulatory signaling domain (e.g., a costimulatory signaling domain described herein) and/or a primary signaling domain (e.g., a primary signaling domain described herein, e.g., a zeta chain described herein).

The present invention also provides vectors in which a nucleic acid molecule encoding a CAR, e.g., a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR is an adenoviral vector (A5/35). In another embodiment, the expression of nucleic acids encoding CARs can be accomplished using of transposons such as sleeping beauty, crisper, CAS9, and zinc finger nucleases. See below June et al. 2009Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EFla promoter. The native EFla promoter drives expression of the alpha subunit of the elongation factor-1 complex, which is responsible for the enzymatic delivery of aminoacyl tRNAs to the ribosome. The EFla promoter has been extensively used in mammalian expression plasmids and has been shown to be effective in driving CAR expression from nucleic acid molecules cloned into a lentiviral vector. See, e.g., Milone et al., Mol. Ther. 17(8): 1453-1464 (2009). In some embodiments, the EFla promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:
(SEQ ID NO: 109)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCGCCTCGTCCTTCGCAGCGGCCCCCCGG
GTGTTCCCATCGCCGCTTCTAGGCCCACTGCGACG
CTTGCCTGCACTTCTTACACGCTCTGGGTCCCAGC
CGCGGCGACGCAAAGGGCCTTGGTGCGGGTCTCGT
CGGCGCAGGGACGCGTTTGGGTCCCGACGGAACCT
TTTCCGCGTTGGGGTTGGGGCACCATAAGCT
Exemplary truncated PGK Promoters:
PGK100:
(SEQ ID NO: 110)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTG
PGK200:
(SEQ ID NO: 111)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACG
PGK300:
(SEQ ID NO: 112)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCG
PGK400:
(SEQ ID NO: 113)
ACCCCTCTCTCCAGCCACTAAGCCAGTTGCTCCCT
CGGCTGACGGCTGCACGCGAGGCCTCCGAACGTCT
TACGCCTTGTGGCGCGCCCGTCCTTGTCCCGGGTG
TGATGGCGGGGTGTGGGGCGGAGGGCGTGGCGGGG
AAGGGCCGGCGACGAGAGCCGCGCGGGACGACTCG
TCGGCGATAACCGGTGTCGGGTAGCGCCAGCCGCG
CGACGGTAACGAGGGACCGCGACAGGCAGACGCTC
CCATGATCACTCTGCACGCCGAAGGCAAATAGTGC
AGGCCGTGCGGCGCTTGGCGTTCCTTGGAAGGGCT
GAATCCCCGCCTCGTCCTTCGCAGCGGCCCCCCGG
GTGTTCCCATCGCCGCTTCTAGGCCCACTGCGACG
CTTGCCTGCACTTCTTACACGCTCTGGGTCCCAGC
CG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a CAR described herein, e.g., a CD19 CAR, and a second CAR, e.g., an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, e.g., mammalian, bacterial, yeast, or insect cell by any method, e.g., one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Natural Killer Cell Receptor (NKR) CARs

In some embodiments, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, K1R2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di Stasa et al., N Engl. J. Med. 2011 Nov. 3; 365(18):1673-1683), can be used as a safety switch in the CAR therapy of the instant invention. In some embodiments, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In some embodiments, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, an RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. In some embodiments, a CAR of the present invention utilizes a dimerization switch as those described in, e.g., WO2014127261, which is incorporated by reference herein. Additional description and exemplary configurations of such regulatable CARs are provided herein and in, e.g., paragraphs 527-551 of International Publication No. WO 2015/090229 filed Mar. 13, 2015, which is incorporated by reference in its entirety. In some embodiments, an RCAR involves a switch domain, e.g., a FKBP switch domain, as set out SEQ ID NO: 114, or comprise a fragment of FKBP having the ability to bind with FRB, e.g., as set out in SEQ ID NO: 115. In some embodiments, the RCAR involves a switch domain comprising a FRB sequence, e.g., as set out in SEQ ID NO: 116, or a mutant FRB sequence, e.g., as set out in any of SEQ ID Nos. 117-122.

(SEQ ID NO: 114)
DVPDYASLGGPSSPKKKRKVSRGVQVETISPGDGRTFPKRGQT
CVVHYTGMLEDGKKFDSSRDRNKPFKFMLGKQEVIRGWEEGVA
QMSVGQRAKLTISPDYAYGATGHPGI1PPHATLVFDVELLKLE
TSY
(SEQ ID NO: 115)
VQVETISPGDGRTFPKRGQTCVVHYTGMLEDGKKFDSSRDRNK
PFKFMLGKQEVIRGWEEGVAQMSVGQRAKLTISPDYAYGATGH
PGIIPPHATLVFDVELLKLETS
(SEQ ID NO: 116)
ILWHEMWHEGLEEASRLYFGERNVKGMFEVLEPLHAMMERGPQ
TLKETSFNQAYGRDLMEAQEWCRKYMKSGNVKDLTQAWDLYYH
VFRRISK

TABLE 2
Exemplary mutant FRB having increased
affinity for a dimerization molecule.

| | | SEQ |
|---|---|---|
| | | ID |
| FRB mutant | Amino Acid Sequence | NO: |
| E2032I mutant | ILWHEMWHEGLIEASRLYFGERNVK | 117 |
| | GMFEVLEPLHAMMERGPQTLKETSF | |
| | NQAYGRDLMEAQEWCRKYMKSGNVK | |
| | DLTQAWDLYYHVERRISKTS | |
| E2032L mutant | ILWHEMWHEGLLEASRLYFGERNVK | 118 |
| | GMFEVLEPLHAMMERGPQTLKETSF | |
| | NQAYGRDLMEAQEWCRKYMKSGNVK | |
| | DLTQAWDLYYHVERRISKTS | |
| 12098L mutant | ILWHEMWHEGLEEASRLYFGERNVK | 119 |
| | GMFEVLEPLHAMMERGPQTLKETSF | |
| | NQAYGRDLMEAQEWCRKYMKSGNVK | |
| | DLLQAWDLYYHVERRISKTS | |
| E2032, 12098 | ILWHEMWHEGLXEASRLYFGERNVK | 120 |
| | GMFEVLEPLHAMMERGPQTLKETSF | |
| mutant | NQAYGRDLMEAQEWCRKYMKSGNVK | |
| | DLXQAWDLYYHVERRISKTS | |
| E20321, 12098L | ILWHEMWHEGLTEASRLYFGERNVK | 121 |
| | GMFEVLEPLHAMMERGPQTLKETSF | |
| mutant | NQAYGRDLMEAQEWCRKYMKSGNVK | |
| | DLLQAWDLYYHVERRISKTS | |
| E2032L, 12098L | ILWHEMWHEGLLEASRLYFGERNVK | 122 |
| | GMFEVLEPLHAMMERGPQTLKETSF | |
| mutant | NQAYGRDLMEAQEWCRKYMKSGNVK | |
| | DLLQAWDLYYHVERRISKTS | |

### RNA Transfection

Disclosed herein are methods for producing an in vitro transcribed RNA CAR. RNA CAR and methods of using the same are described, e.g., in paragraphs 553-570 of in International Application WO2015/142675, filed Mar. 13, 2015, which is herein incorporated by reference in its entirety.

An immune effector cell can include a CAR encoded by a messenger RNA (mRNA). In some embodiments, the mRNA encoding a CAR described herein is introduced into an immune effector cell, e.g., made by a method described herein, for production of a CAR-expressing cell.

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (e.g., a hinge region described herein), a transmembrane domain (e.g., a transmembrane domain described herein such as a transmembrane domain of CD8a); and a cytoplasmic region that includes an intracellular signaling domain, e.g., an intracellular signaling domain described herein, e.g., comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 31) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines (e.g., SEQ ID NO: 33).

Poly(A) tails of RNAs can be further extended following in vitro transcription with the use of a poly(A) polymerase, such as E. coli polyA polymerase (E-PAP). In some embodiments, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some embodiments, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

Methods of Manufacture/Production

In some embodiments, the methods disclosed herein further include administering a T cell depleting agent after treatment with the cell (e.g., an immune effector cell as described herein), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In some embodiments, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In some embodiments, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In some embodiments, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19-expressing normal cells or CD19-expressing cancer cells.

### Elutriation

In some embodiments, the methods described herein feature an elutriation method that removes unwanted cells, e.g., monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, e.g., a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, e.g., cell sample, e.g., thawed cell sample, by dilution with certain isotonic solutions (e.g., PBS), and using an optimized combination of flow rates and collection volume for each fraction collected by an elutriation device. Exemplary elutriation methods that could be applied in the present invention are described on pages 48-51 of WO 2017/117112, herein incorporated by reference in its entirety.

### Density Gradient Centrifugation

Manufacturing of adoptive cell therapeutic product requires processing the desired cells, e.g., immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, e.g., as described in the Examples herein. For example, a loss of T cells in the final product, with a relative gain of non-T cells, especially undesirable B cells, blast cells and monocytes, was observed in cell preparations isolated by density gradient centrifugation through Ficoll solution.

Without wishing to be bound by theory, it is believed that immune effector cells, e.g., T cells, dehydrate during cryopreservation to become denser than fresh cells. Without wishing to be bound by theory, it is also believed that immune effector cells, e.g., T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, e.g., 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, e.g., greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, e.g., selection for the desired immune effector cells. In another embodiment, the selection comprises a negative selection, e.g., selection for unwanted cells, e.g., removal of unwanted cells. In embodiments, the positive or negative selection methods described herein are performed under flow conditions, e.g., by using a flow-through device, e.g., a flow-through device described herein. Exemplary positive and negative selections are described on pages 53-57 of WO 2017/117112, herein incorporated by reference in its entirety. Selection methods can be performed under flow conditions, e.g., by using a flow-through device, also referred to as a cell processing system, to further enrich a preparation of cells for desired immune effector cells, e.g., T cells, suitable for CAR expression. Exemplary flow-through devices are described on pages 57-70 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS Plus or CliniMACS Prodigy) can be used.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion.

In one such process, illustrated here by way of example, cells, for example, T cells, are collected from a donor (for example, a patient to be treated with an autologous chimeric antigen receptor T cell product) via apheresis (e.g., leukapheresis). Collected cells may then be optionally purified, for example, by an elutriation step, or via positive or negative selection of target cells (e.g., T cells). The process may also include a transduction step, wherein nucleic acid encoding one or more desired proteins, for example, a CAR, for example a CAR targeting CD19, is introduced into the cell. The nucleic acid may be introduced in a lentiviral vector. The cells, e.g., the lentivirally transduced cells, may then be expanded for a period of days, for example, 1, 2, 3, 4, 5, 6, 7, 8, 9, 10, or more days, for example in the presence of a suitable medium. Examples of CAR T cells and their manufacture are further described, for example, in WO2012/079000, which is incorporated herein by reference in its entirety. The systems and methods of the present disclosure may be used for any cell separation/purification processes described in or associated with WO2012/079000. Additional CAR T manufacturing processes are described in, e.g., WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate cells to produce a cell product.

Additional manufacturing methods and processes that can be combined with the present invention have been described in the art. For examples, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, e.g., T cells, and removing unwanted materials, e.g., unwanted cells. The additional methods or steps described in this section can be used in combination with any of the elutriation, density gradient centrifugation, selection under flow conditions, or improved wash step described in the preceding sections.

A source of cells, e.g., T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present disclosure, immune effector cells, e.g., T cells, can be obtained from a unit of blood collected from a subject using any number of techniques known to the skilled artisan, and any of the methods disclosed herein, in any combination of steps thereof. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. In another embodiment, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In some embodiments, desired immune effector cells, e.g., T cells, are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, e.g., CD25+ depleted cells or CD25^{high} depleted cells, using, e.g., a negative selection technique, e.g., described herein. In some embodiments, the population of T regulatory-depleted cells contains less than 30%, 25%, 20%, 15%, 10%, 5%, 4%, 3%, 2%, 1% of CD25+ cells or CD25^{high} cells.

In some embodiments, T regulatory cells, e.g., CD25+ T cells or CD25^{high} T cells, are removed from the population using an anti-CD25 antibody, or fragment thereof, or a CD25-binding ligand, e.g. IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells or CD25^{high} T cells, are removed from the population using CD25 depleting reagent from Miltenyi™. In some embodiments, the ratio of cells to CD25 depletion reagent is 1×10⁷ cells to 20 μL, or 1×10⁷ cells to 15 μL, or 1×10⁷ cells to 10 μL, or 1×10⁷ cells to 5 μL, or 1×10⁷ cells to 2.5 μL, or 1×10⁷ cells to 1.25 μL. In some embodiments, e.g., for T regulatory cells, greater than 500 million cells/mL is used. In a further aspect, a concentration of cells of 600, 700, 800, or 900 million cells/mL is used.

In some embodiments, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In some embodiments, the resulting population T regulatory-depleted cells has 2×10⁹ T regulatory cells, e.g., CD25+ cells or CD25^{high} cells, or less (e.g., 1×10⁹, 5×10⁸, 1×10⁸, 5×10⁷, 1×10⁷, or less T regulatory cells).

In some embodiments, the T regulatory cells, e.g., CD25+ cells or CD25^{high} cells, are removed from the population using the CliniMAC system with a depletion tubing set, such as, e.g., tubing 162-01. In some embodiments, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) Treg cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete Treg cells prior to manufacturing of the CAR-expressing cell (e.g., T cell, NK cell) product.

Without wishing to be bound by a particular theory, decreasing the level of negative regulators of immune cells (e.g., decreasing the number of unwanted immune cells, e.g., Treg cells), in a subject prior to apheresis or during manufacturing of a CAR-expressing cell product can reduce the risk of a subject's relapse. In some embodiments, a subject is pre-treated with one or more therapies that reduce Treg cells prior to collection of cells for CAR-expressing cell product manufacturing, thereby reducing the risk of subject relapse to CAR-expressing cell treatment. In some embodiments, methods of decreasing Treg cells include, but are not limited to, administration to the subject of one or more of cyclophosphamide, anti-GITR antibody, CD25-depletion, or a combination thereof. In some embodiments, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (e.g., depleting) Treg cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete Treg cells prior to manufacturing of the CAR-expressing cell (e.g., T cell, NK cell) product.

In some embodiments, a subject is pre-treated with cyclophosphamide prior to collection of cells for CAR-expressing cell product manufacturing, thereby reducing the risk of subject relapse to CAR-expressing cell treatment (e.g., CTL019 treatment). In some embodiments, a subject is pre-treated with an anti-GITR antibody prior to collection of cells for CAR-expressing cell (e.g., T cell or NK cell) product manufacturing, thereby reducing the risk of subject relapse to CAR-expressing cell treatment.

In some embodiments, the CAR-expressing cell (e.g., T cell, NK cell) manufacturing process is modified to deplete Treg cells prior to manufacturing of the CAR-expressing cell (e.g., T cell, NK cell) product (e.g., a CTL019 product). In some embodiments, CD25-depletion is used to deplete Treg cells prior to manufacturing of the CAR-expressing cell (e.g., T cell, NK cell) product (e.g., a CTL019 product).

In some embodiments, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory-depleted, e.g., CD25+ depleted or CD25^{high} depleted, and tumor antigen depleted cells that are suitable for expression of a CAR, e.g., a CAR described herein. In some embodiments, tumor antigen expressing cells are removed simultaneously with the T regulatory, e.g., CD25+ cells or CD25^{high} cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells or CD25^{high} cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory-depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta), e.g., as described herein. In some embodiments, check point inhibitor expressing cells are removed simultaneously with the T regulatory, e.g., CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells or CD25^{high} cells, and the removal of the check point inhibitor expressing cells is sequential, and can occur, e.g., in either order.

In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in some embodiments, a concentration of 10 billion cells/mL, 9 billion cells/mL, 8 billion cells/mL, 7 billion cells/mL, 6 billion cells/mL, or 5 billion cells/mL is used. In some embodiments, a concentration of 1 billion cells/mL is used. In some embodiments, a concentration of cells from 75, 80, 85, 90, 95, or 100 million cells/mL is used. In further aspects, concentrations of 125 or 150 million cells/mL can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In other aspects, the concentration used can be from about 1×10⁵/mL to 1×10⁶/mL, and any integer value in between.

In other aspects, the cells may be incubated on a rotator for varying lengths of time at varying speeds at either 2-10° C. or at room temperature.

In some embodiments, a plurality of the immune effector cells of the population do not express diaglycerol kinase (DGK), e.g., is DGK-deficient. In some embodiments, a plurality of the immune effector cells of the population do not express Ikaros, e.g., is Ikaros-deficient. In some embodiments, a plurality of the immune effector cells of the population do not express DGK and Ikaros, e.g., is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In some embodiments a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the immune effector cells expressing a CAR molecule, e.g., a CAR molecule described herein, are obtained from a subject that has received a low, immune enhancing dose of an mTOR inhibitor. In some embodiments, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one embodiment, the methods of the application can utilize culture media conditions comprising serum-free medium. In one embodiment, the serum free medium is OpTmizer CTS (LifeTech), Immunocult XF (Stemcell technologies), CellGro (CellGenix), TexMacs (Miltenyi), Stemline (Sigma), Xvivol5 (Lonza), PrimeXV (Irvine Scientific), or StemXVivo (RandD systems).

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In another embodiment, the NK cells are an NK cell line, e.g., NK-92 cell line (Conkwest).

### Allogeneic CAR-Expressing Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta). Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

Expression systems for siRNA and shRNAs, and exemplary shRNAs, are described, e.g., in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

The CRISPR/Cas system, and uses thereof, are described, e.g., in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

TALENs and uses thereof, are described, e.g., in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

ZFNs, and uses thereof, are described, e.g., in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386, Aug. 28 2008), and may be even shorter in manufactured CAR-expressing cells, e.g., CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in some embodiments, an immune effector cell, e.g., a T cell, ectopically expresses a telomerase subunit, e.g., the catalytic subunit of telomerase, e.g., TERT, e.g., hTERT. In some embodiments, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

Telomerase expression may be stable (e.g., the nucleic acid may integrate into the cell's genome) or transient (e.g., the nucleic acid does not integrate, and expression declines after a period of time, e.g., several days). Stable expression may be accomplished by transfecting or transducing the cell with DNA encoding the telomerase subunit and a selectable marker, and selecting for stable integrants. Alternatively or in combination, stable expression may be accomplished by site-specific recombination, e.g., using the Cre/Lox or FLP/FRT system.

Transient expression may involve transfection or transduction with a nucleic acid, e.g., DNA or RNA such as mRNA. In some embodiments, transient mRNA transfection avoids the genetic instability sometimes associated with stable transfection with TERT. Transient expression of exogenous telomerase activity is described, e.g., in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. Nos. 8,710,200, 8,822,663, 8,680,069, 8,754,062, 8,664,194, or 8680069.

In some embodiments, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 108)
MPRAPRCRAVRSLLRSHYREVLPLATFVRRLGPQGW
RLVQRGDPAAFRALVAQCLVCVPWDARPPPAAPSF
RQVSCLKELVARVLQRLCERGAKNVLAFGFALLDG
ARGGPPEAFTTSVRSYLPNTVTDALRGSGAWGLLL
RRVGDDVLVHLLARCALFVLVAPSCAYQVCGPPLY
QLGAATQARPPPHASGPRRRLGCERAWNHSVREAG
VPLGLPAPGARRRGGSASRSLPLPKRPRRGAAPEP
ERTPVGQGSWAHPGRTRGPSDRGFCVVSPARPAEE
ATSLEGALSGTRHSHPSVGRQHHAGPPSTSRPPRP
WDTPCPPVYAETKHFLYSSGDKEQLRPSFLLSSLR
PSLTGARRLVETIFLGSRPWMPGTPRRLPRLPQRY
WQMRPLFLELLGNHAQCPYGVLLKTHCPLRAAVTP
AAGVCAREKPQGSVAAPEEEDTDPRRLVQLLRQHS
SPWQVYGFVRACLRRLVPPGLWGSRHNERRFLRNT
KKFISLGKHAKLSLQELTWKMSVRGCAWLRRSPGV
GCVPAAEHRLREEILAKFLHWLMSVYVVELLRSFF
YVTETTFQKNRLFFYRKSVWSKLQSIGIRQHLKRV
QLRELSEAEVRQHREARPALLTSRLRFIPKPDGLR
PIVNMDYVVGARTFRREKRAERLTSRVKALFSVLN
YERARRPGLLGASVLGLDDIHRAWRTFVLRVRAQD
PPPELYFVKVDVTGAYDTIPQDRLTEVIASIIKPQ
NTYCVRRYAVVQKAAHGHVRKAFKSHVSTLTDLQP
YMRQFVAHLQETSPLRDAVVIEQSSSLNEASSGLF
DVFLRFMCHHAVRIRGKSYVQCQGIPQGSILSTLL
CSLCYGDMENKLFAGIRRDGLLLRLVDDFLLVTPH
LTHAKTFLRTLVRGVPEYGCVVNLRKTVVNFPVED
EALGGTAFVQMPAHGLFPWCGLLLDTRTLEVQSDY
SSYARTSIRASLTFNRGFKAGRNMRRKLFGVLRLK
CHSLFLDLQVNSLQTVCTNIYKILLLQAYRFHACV
LQLPFHQQVWKNPTFFLRVISDTASLCYSILKAKN
AGMSLGAKGAAGPLPSEAVQWLCHQAFLLKLTRHR
VTYVPLLGSLRTAQTQLSRKLPGTTLTALEAAANP
ALPSDFKTILD

In some embodiments, the hTERT has a sequence at least 80%, 85%, 90%, 95%, 96%, 97%, 98%, or 99% identical to the sequence of SEQ ID NO: 108. In some embodiments, the hTERT has a sequence of SEQ ID NO: 108. In some embodiments, the hTERT comprises a deletion (e.g., of no more than 5, 10, 15, 20, or 30 amino acids) at the N-terminus, the C-terminus, or both. In some embodiments, the hTERT comprises a transgenic amino acid sequence (e.g., of no more than 5, 10, 15, 20, or 30 amino acids) at the N-terminus, the C-terminus, or both.

In some embodiments, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### Pharmaceutical Compositions and Treatments

In some embodiments, the disclosure provides a method of treating a patient, comprising administering CAR-expressing cells produced as described herein, optionally in combination with one or more other therapies. In some embodiments, the disclosure provides a method of treating a patient, comprising administering a reaction mixture comprising CAR-expressing cells as described herein, optionally in combination with one or more other therapies. In some embodiments, the disclosure provides a method of shipping or receiving a reaction mixture comprising CAR-expressing cells as described herein. In some embodiments, the disclosure provides a method of treating a patient, comprising receiving a CAR-expressing cell that was produced as described herein, and further comprising administering the CAR-expressing cell to the patient, optionally in combination with one or more other therapies. In some embodiments, the disclosure provides a method of treating a patient, comprising producing a CAR-expressing cell as described herein, and further comprising administering the CAR-expressing cell to the patient, optionally in combination with one or more other therapies. The other therapy may be, e.g., a cancer therapy such as chemotherapy.

In some embodiments, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In some embodiments, a therapy described herein, e.g., a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In some embodiments, the GITR binding molecules and/or molecules modulating GITR functions (e.g., GITR agonist and/or Treg depleting GITR antibodies) are administered prior to the CAR-expressing cell. For example, in some embodiments, a GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In some embodiments, the subject has cancer (e.g., a solid cancer or a hematological cancer such as ALL or CLL). In some embodiments, the subject has CLL. In embodiments, the subject has ALL. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In some embodiments, a CAR expressing cell described herein is administered to a subject in combination with a GITR agonist, e.g., a GITR agonist described herein. In some embodiments, the GITR agonist is administered prior to the CAR-expressing cell. For example, in some embodiments, the GITR agonist can be administered prior to apheresis of the cells. In some embodiments, the subject has CLL.

The methods described herein can further include formulating a CAR-expressing cell in a pharmaceutical composition. Pharmaceutical compositions may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated, e.g., for intravenous administration.

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises about 1.1×10⁶-1.8×10⁷ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, e.g., by intradermal or subcutaneous injection. The compositions of immune effector cells (e.g., T cells, NK cells) may be injected directly into a tumor, lymph node, or site of infection.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Rapid Manufacturing of Potent Quiescent CAR-T Cells in Less than 24 Hours Using Lentiviral Vectors

Current CAR-T cell therapies rely upon manufacturing approaches that include ex vivo culture over days to weeks that is associated with significant labor and material costs. Described herein is a simple and rapid process for generation of CAR-T cells using lentiviral vector transduction that can be completed within less than 24 hours of T cell collection. In a murine xenograft model of acute lymphoblastic leukemia, CD19-specific CAR-T cells generated using this ultrashort manufacturing process exhibit potent, dose-dependent anti-leukemic activity associated with persistent engraftment and durable anti-leukemic activity.

Adoptive cellular immunotherapy (ACT) using T cells that are genetically modified to express a chimeric antigen receptor (CAR) or cloned T cell receptor (TCR) induce robust anti-tumor responses in patients with hematologic malignancies (Brentjens, R. J. et al. Science translational medicine 5, 177ra138 (2013); Grupp, S. A. et al. The New England journal of medicine 368, 1509-1518 (2013); Kalos, M. et al. Science translational medicine 3 (2011); Maude, S. L. et al. The New England journal of medicine 371, 1507-1517 (2014); Porter, D. L., The New England journal of medicine 365, 725-733 (2011); Porter, D. L. et al. Science translational medicine 7, 303ra139 (2015)). T cell engraftment following adoptive transfer is associated with both the depth and the duration of clinical response (Porter, D. L. et al. Science translational medicine 7, 303ra139 (2015); Maude, S. L. et al. Blood 125, 4017-4023 (2015); Kochenderfer, J. N. et al. J Clin Oncol, JCO2016713024 (2017)). Despite the high overall rate of complete response to CD19-specific CAR therapy in acute lymphoblastic leukemia (ALL), some patients still relapse due to premature loss of the engineered T cells (Maude, S. L. et al. The New England journal of medicine 371, 1507-1517 (2014)). The ability of T cells to engraft following adoptive transfer is related to their state of differentiation (Berger, C. et al. The Journal of clinical investigation 118, 294-305 (2008); Graef, P. et al. Immunity 41, 116-126 (2014); Hinrichs, C. S. et al. Proceedings of the National Academy of Sciences of the United States of America 106, 17469-17474 (2009); Hinrichs, C. S. et al. Blood 117, 808-814 (2011)). Following activation, T cells enter a phase of rapid proliferation that is associated with progressive differentiation (Ghassemi, S. et al. Cancer Immunol Res 6, 1100-1109 (2018)). A number of approaches have been developed to limit activation-induced differentiation including inhibition of Fas-FasL interactions (Klebanoff, C. A. et al. J Clin Invest 126, 318-334 (2016)), inhibition of Akt signaling (Crompton, J. G. et al. Cancer Res 75, 296-305 (2015); van der Waart, A. B. et al. Blood 124, 3490-3500 (2014)) or alternatively activating Wnt signaling (Gattinoni, L. et al. Nature medicine 15, 808-813 (2009); Gattinoni, L. et al. Nat Med 17, 1290-1297 (2011); Muralidharan, S. et al. The Journal of Immunology 187, 5221-5232 (2011)). While approaches to limiting T cell differentiation during ex vivo expansion may aid engraftment following adoptive transfer, eliminating the stimulation and ex vivo culture of T cells offers a potentially far simpler and more cost-effective approach provided the barriers to gene transfer into quiescent T cells can be overcome.

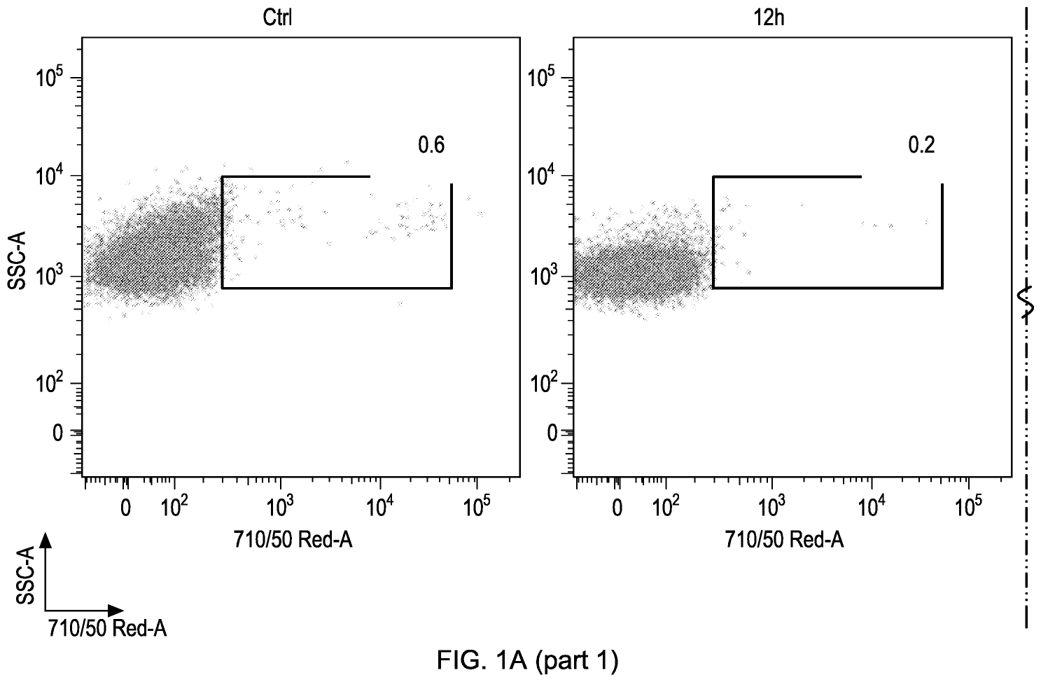

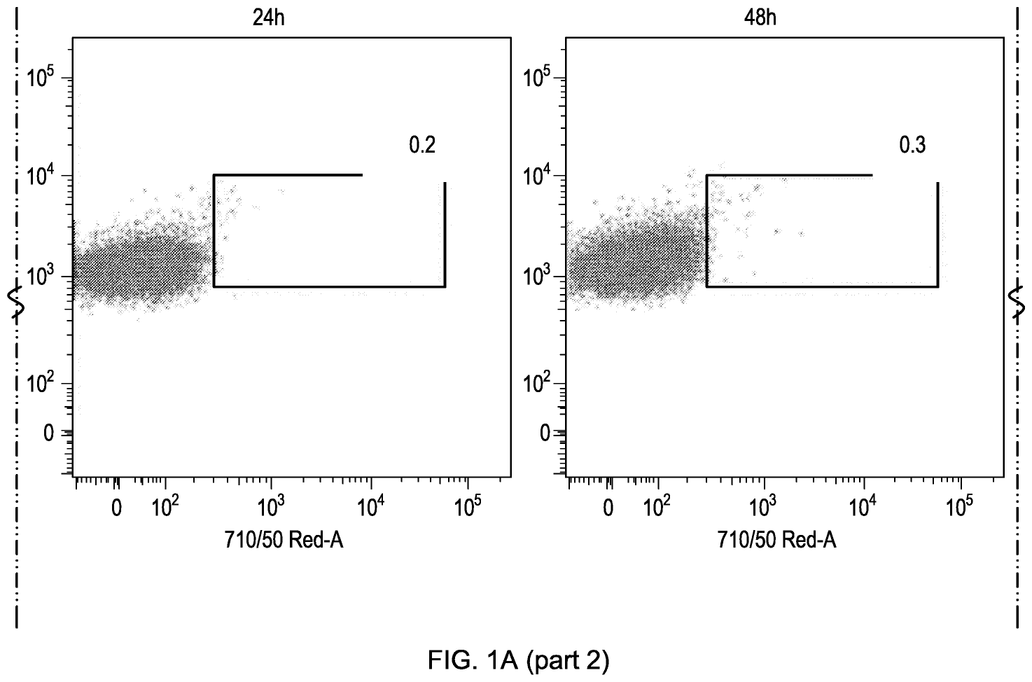

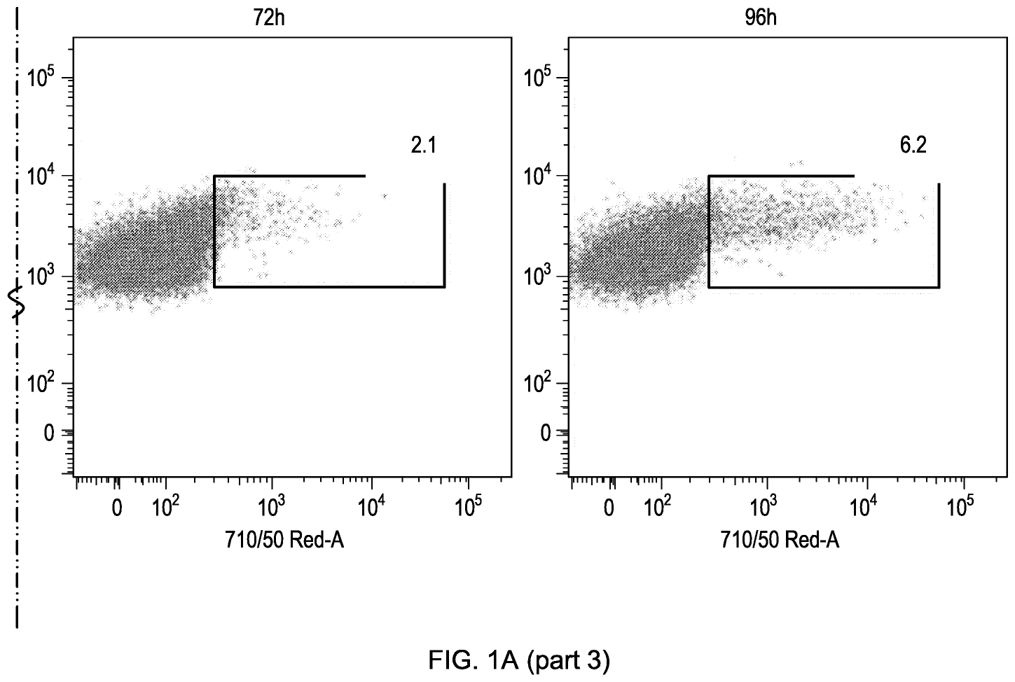

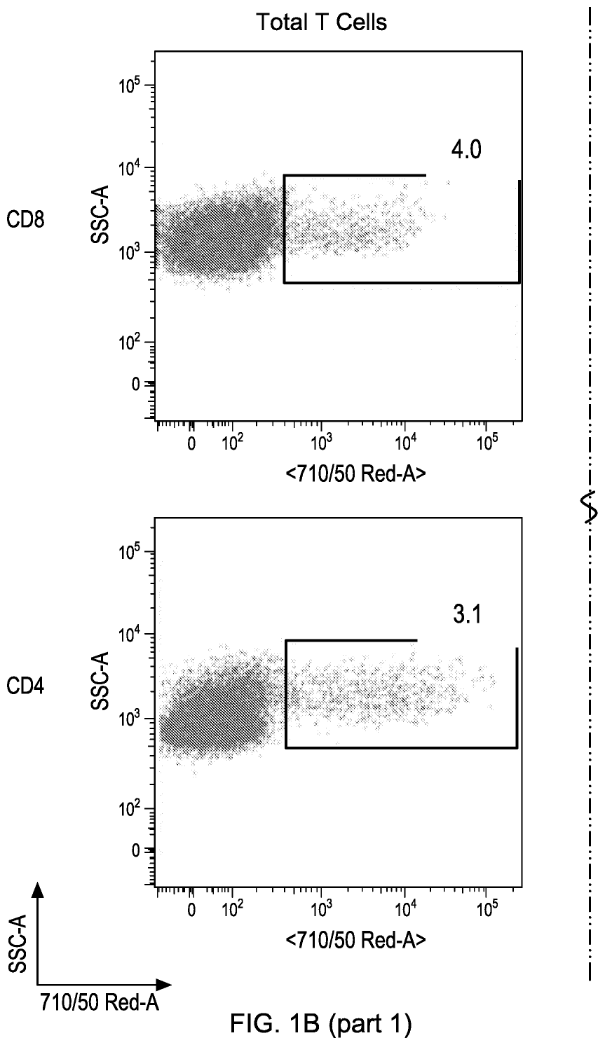

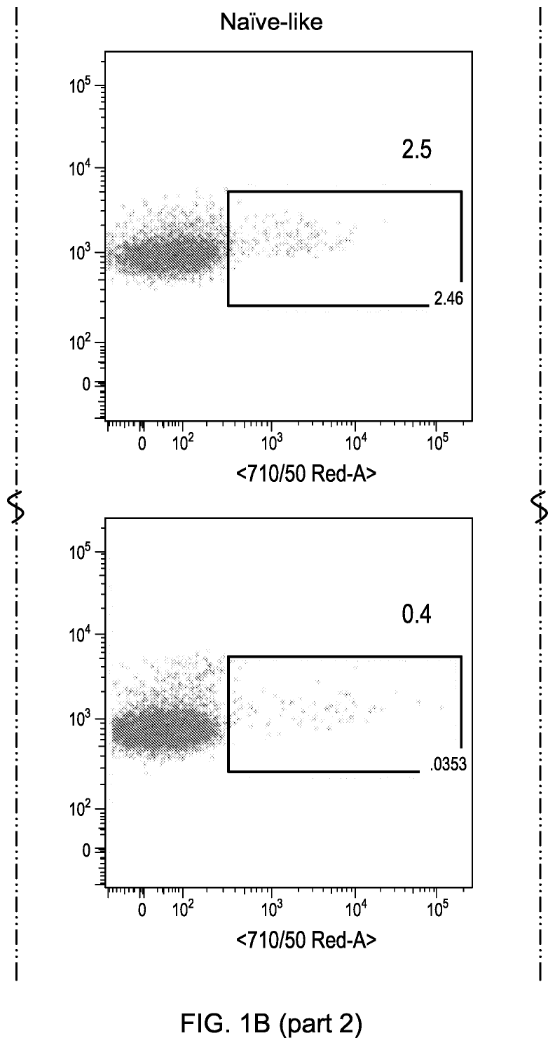

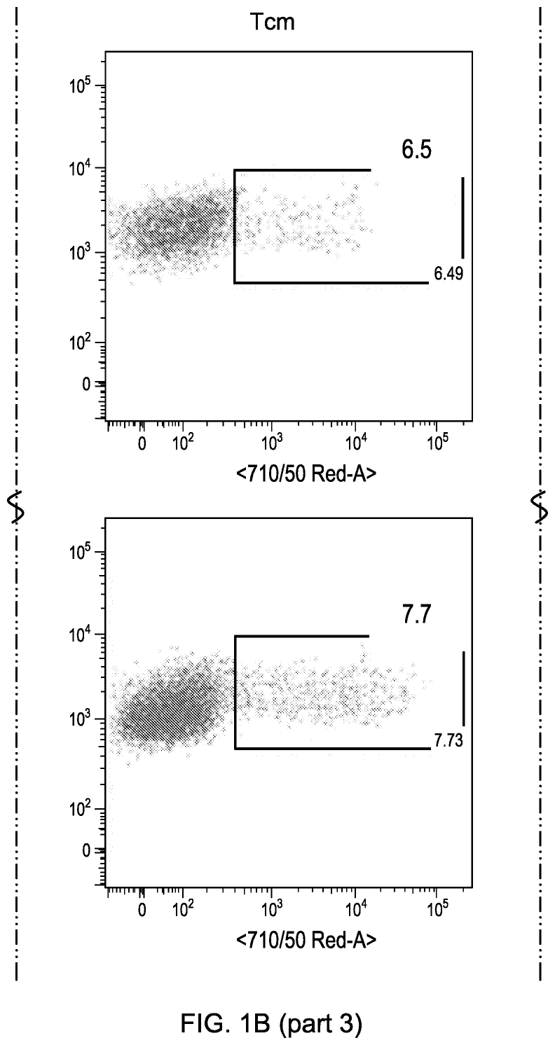

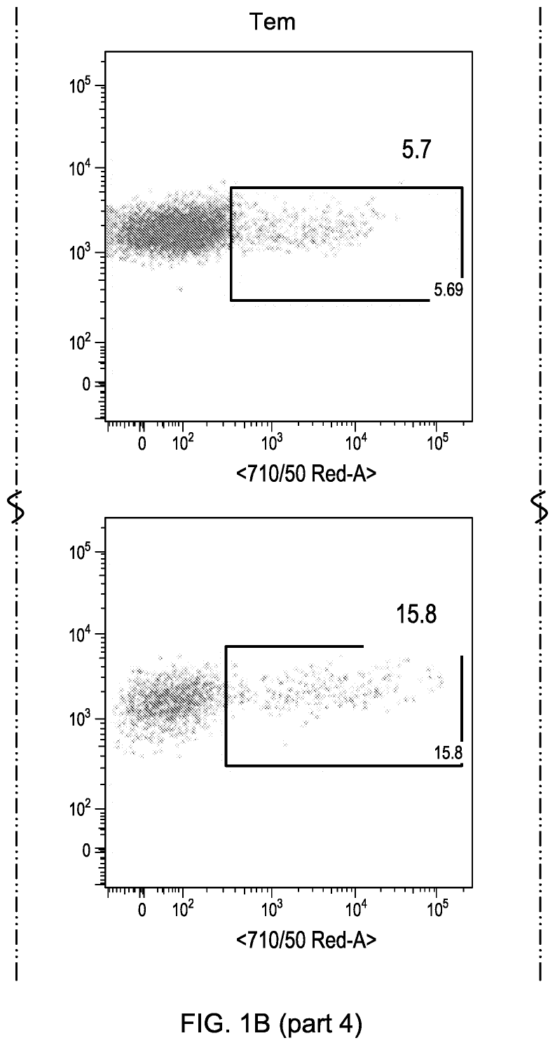

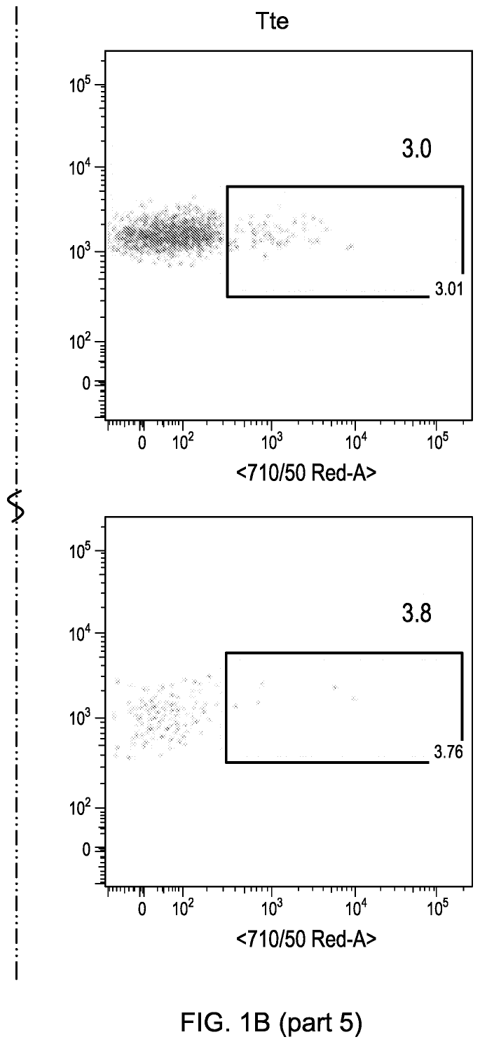

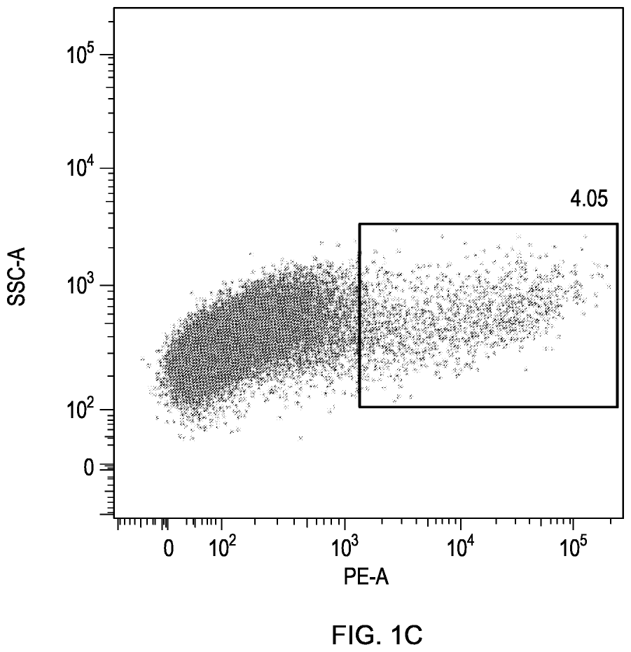

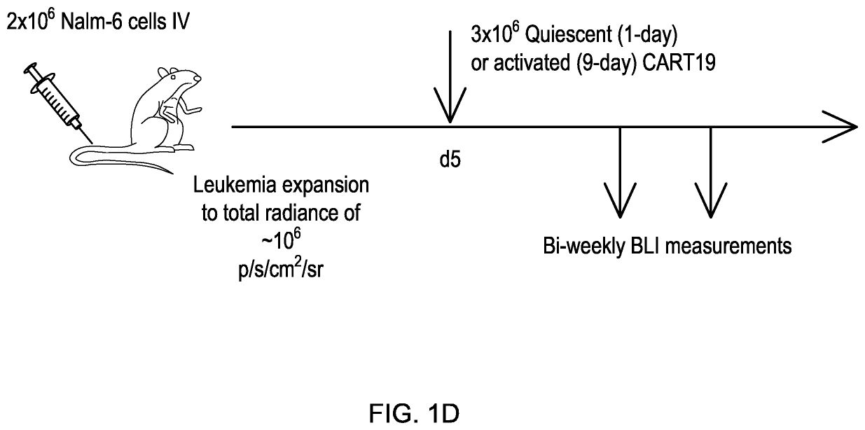

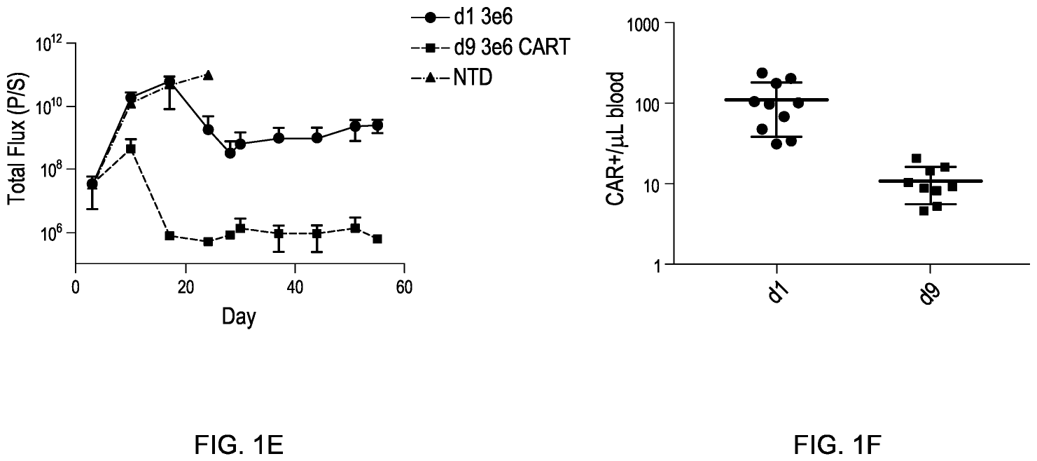

Natural HIV has the ability to infect quiescent T cells in the G0 stage of the cell cycle (Naldini, L. et al. Science 272, 263-267 (1996); Plesa, G. et al. J Virol 81, 13938-13942 (2007); Swiggard, W. J. et al. J Virol 79, 14179-14188 (2005)). Unlike gamma-retroviruses, HIV-derived lentiviral vectors are capable of transducing both dividing and non-dividing cells. IL-7 can enhance the transduction efficiency of quiescent T cells (Cavalieri, S. et al. Blood 102, 497-505 (2003)). However, transduction efficiency of quiescent T cells is reported to be relatively low compared with activated T cells (Korin, Y. D. & Zack, J. A. J Virol 73, 6526-6532 (1999); Rausell, A. et al. Retrovirology 13, 43 (2016)). This low transduction efficiency was confirmed using a 3rd generation lentiviral vector encoding an infrared fluorescent protein as shown in FIG. 1A. In addition to low overall efficiency, the kinetics of transduction of quiescent T cells is also slower than activated T cells, requiring at least 96 hours to achieve detectable expression of the iRFP transgene compared with less than 48 hours for activated T cells. This slower kinetics is consistent with the slower rate of reverse transcription reported for natural HIV in quiescent T cells compared with activated T cells (Pierson, T. C. et al. J Virol 76, 8518-8531 (2002)). Transduction was also observed in both memory and naive subsets of CD4+ and CD8+ T cells with the highest efficiency in cells with a memory phenotype (FIG. 1B).

Despite the low efficiency and slower kinetics of transduction, the in vivo anti-leukemic activity of CD19-specific CAR engineered T cells (CART19) was evaluated. These cells were generated by the simple mixing of freshly isolated, quiescent human T cells with a CAR-encoding lentiviral vector for 16 hours under static culture conditions followed by washing and injection into tumor bearing mice. Similar to experiments with an iRFP transgene, ˜4% of these transduced T cells maintained in medium containing IL-7 and IL-15 for 16 hours, and then activated through their CAR. They exhibited detectable CAR expression (FIG. 1C). Despite this low transduction efficiency, 2×10⁶ T cells transduced using this approach partially controlled the growth of established Nalm6 leukemia for more than 1 month (FIGS. 1D and 1E). This sustained control of Nalm6 growth was associated with persistent engraftment of CAR+ cells (FIG. 1F) indicating that the low number of CAR+ T cells transferred into these mice, estimated at ˜120,000 cells based upon the ˜4% transduction efficiency, had extensive replicative capacity required to maintain CART cell activity in the setting of the rapidly proliferating Nalm6 leukemic cells.

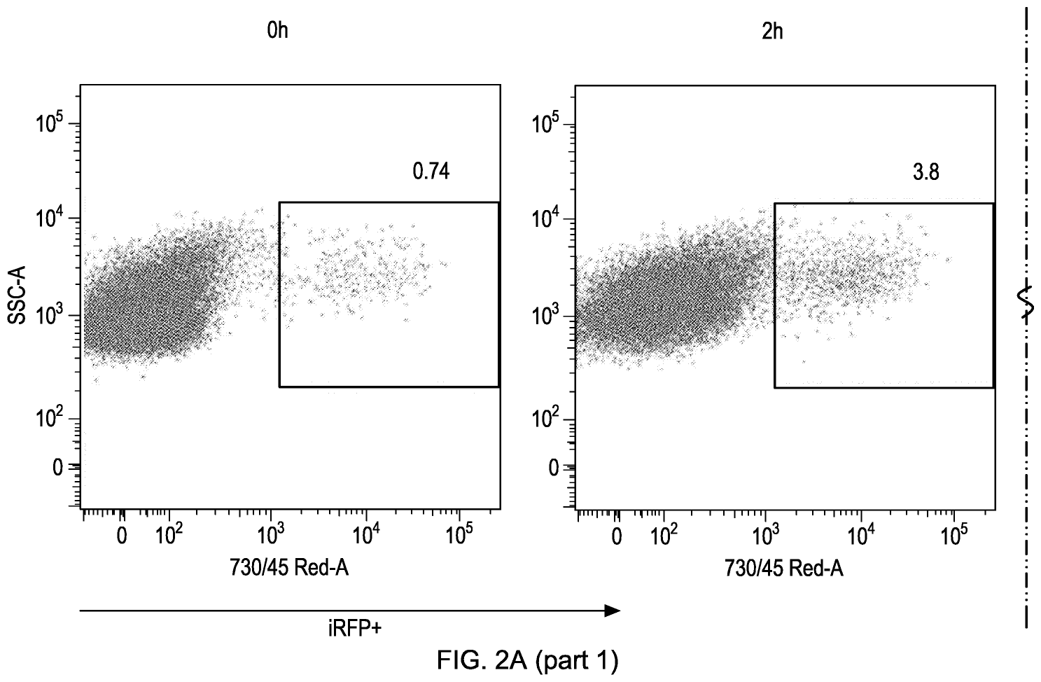

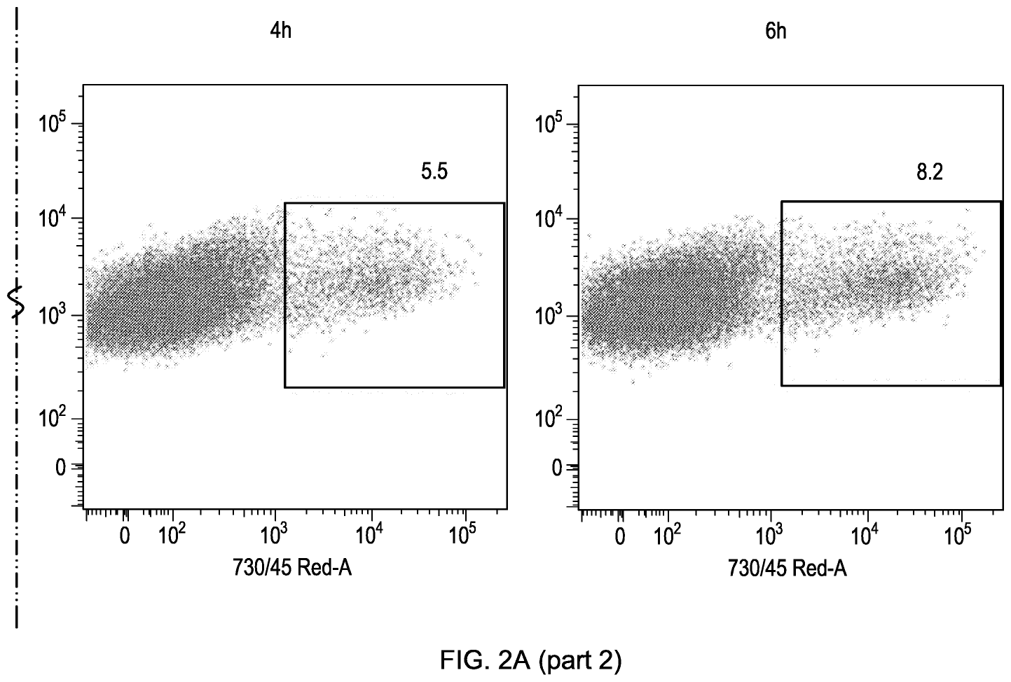

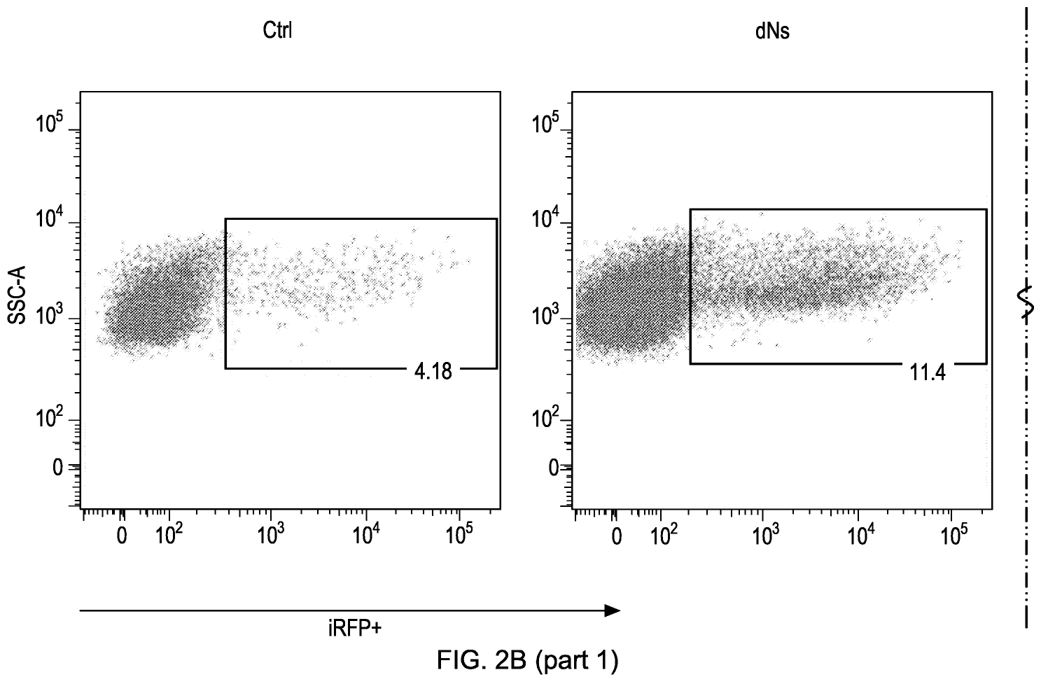

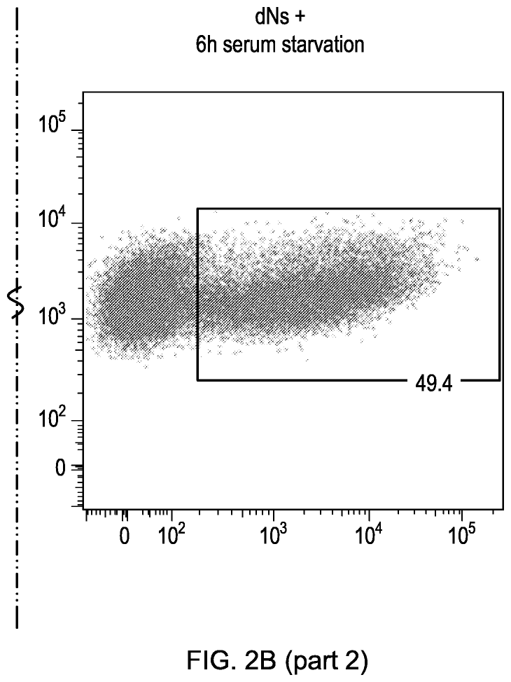

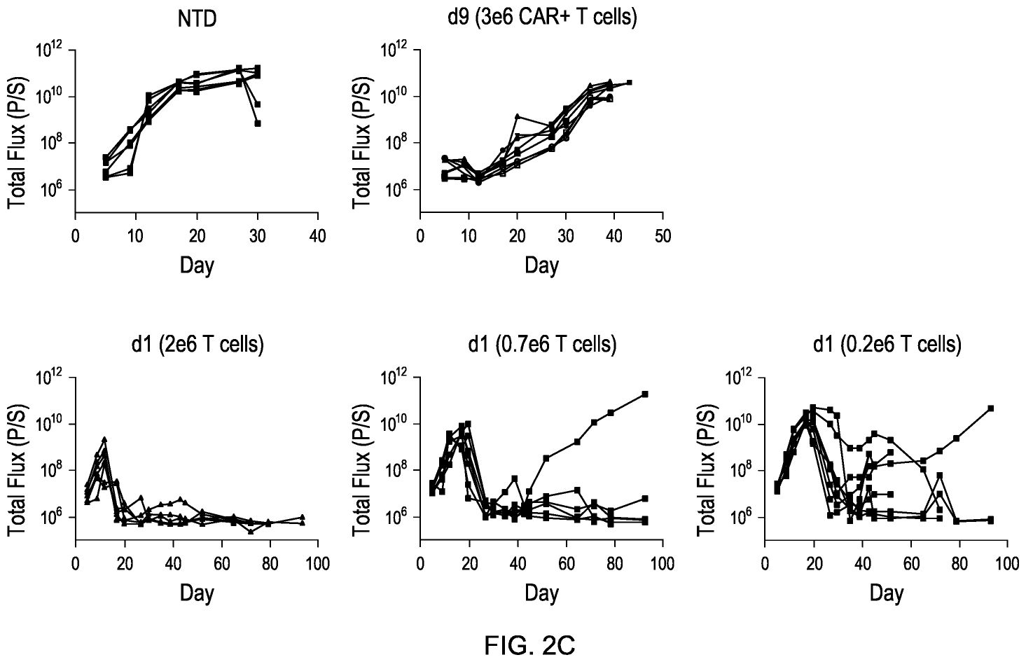

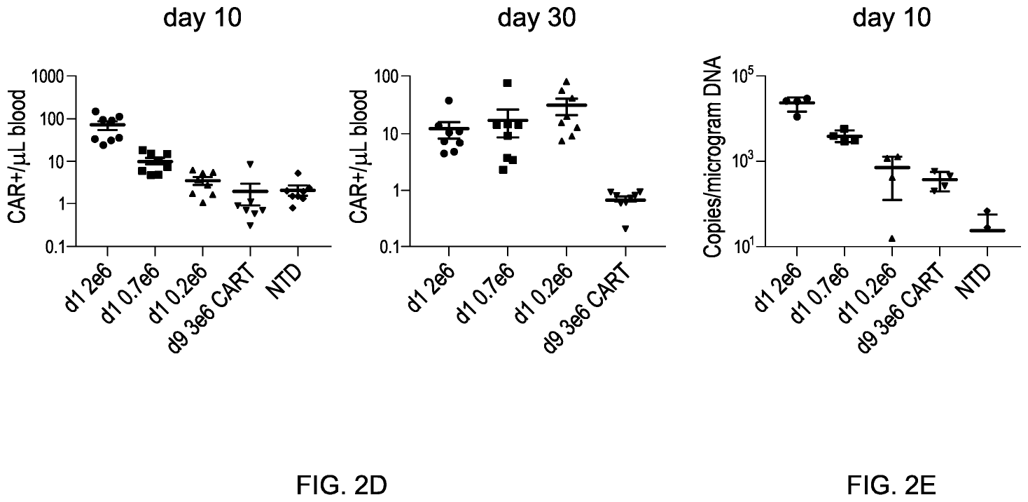

The sustained anti-leukemic activity observed in the context of a low number of transduced T cells suggested that improving the efficiency of transduction should enhance the potency of CART19 cells generated from quiescent T cells. It was evaluated whether a brief serum starvation prior to lentiviral vector transduction would increase lentiviral vector transduction of quiescent T cells. As shown in FIG. 2A, as little as 2 hours of serum starvation increases the transduction of quiescent T cells with about 10-fold increase achieved after 6 hours.

The slow kinetics of reverse transcription in quiescent T cells by both natural HIV and lentiviral vectors may also contribute to reduced transduction efficiency. As shown in FIG. 2B, supplementation of the culture medium with deoxynucleosides (dNs), which enhance reverse transcription, at a 50 μM concentration increases transduction efficiency of quiescent T cells by 3-fold compared with cells maintained in medium containing IL-7 and IL-15. Combining both serum starvation and high concentrations of dNs permits transduction efficiencies exceeding 50% in quiescent T cells (FIG. 2B).

To evaluate the potency of CART19 cells generated using the optimized transduction method, an in vivo functional “CAR stress test” was performed using limited numbers of CART cells in the established Nalm6 leukemia model similar to that used to evaluate CART cells generated by vector-free gene editing approaches (Brown, M. S. & Goldstein, J. L. Science 232, 34-47 (1986)). Following a 2-hr serum starvation of freshly isolated T cells followed by 20 hours of transduction in the presence of 50 μM dNs, the transduced T cells were washed to remove remaining free vectors and 2×10⁶, 7×10⁵ or 2×10⁵ total cells were injected in NSG mice bearing pre-established Nalm6 xenografts. 3×10⁶ CART19 cells prepared by CD3/CD28 bead stimulation followed by lentiviral transduction and 9 days of expansion were used as a control as well as 3×10⁶ mock transduced quiescent T cells. As shown in FIG. 2C, complete regression of Nalm6 leukemia was observed in all CAR-expressing groups. The kinetics of the anti-leukemic response for the CART19 cells generated using quiescent T cells was dose dependent with a median time to complete response for the lowest dose group of 30 days compared with 20 days for the highest dose group. The CD3/CD28 stimulated and expanded CART19 cells showed the most rapid leukemia clearance. However, the durability of the response for this particular donor was limited with all mice ultimately relapsing. In contrast, CART19 cells manufactured from quiescent T cells maintained control of leukemia to below detectable levels by bioluminescence imaging for the duration of the experiment, which included >1 month in the highest dose group. This sustained control of Nalm6 growth was associated with persistent engraftment of CAR+ cells (FIG. 2D-2E). The observed durability of CAR-T cell function in the Nalm6 preclinical xenograft model suggests that engineering of quiescent T cells may improve the overall quality of T cells, perhaps by eliminating the effector differentiation associated with ex vivo culture following CD3 and CD28 stimulation. Studies in syngeneic murine models suggest that memory T cell subsets, particularly the central memory and stem cell memory subsets, may be the most optimal subsets for adoptive T cell therapy. However, these studies may depend upon the primary and secondary costimulatory signals available. As the signals required to promote proliferation and differentiation of T cells likely differ across T cell subsets, it is possible that alternative CAR designs may be required for maximally potent therapies based upon quiescent T cells.

In summary, the ability to generate genetically-modified T cells with superior therapeutic potential in a shorter time period has important implications for CAR-T manufacturing and therapy. Our results indicate that extended ex vivo culture is unnecessarily costly and redundant as an effective CAR-T cell product with durable engraftment in vivo can be generated in as little as 1 day using lentiviral vectors by taking advantage of their unique ability to infect and integrate into the genome of non-dividing cells. In addition to reducing labor, the simple manufacturing methods used in the studies are highly amenable to automation. The more limited ex vivo manipulation also conserves limited resources such as human serum and manufacturing space as expansion occurs entirely in vivo. There are also potential regulatory advantages as well since current FDA guidelines require replication competent lentivirus/retrovirus (PCR/RCL) testing for culture periods greater than 96 hours post transduction (Guidance for Industry, Hum Gene Ther 12, 315-320 (2001)) Eliminating the need for culture will avoid the need for this expensive testing. Finally, more rapid product generation will lead to a shorter period of time between apheresis collection and re-infusion of product T cells that may be important to feasibility of treating patients, especially with rapidly progressive disease (Couzin-Frankel, J. Science 356, 1112-1113 (2017)).

### Materials and Methods

### Generation of CAR Constructs

The CD19-BBζ CAR consisting of a CD8 hinge, 4-1BB costimulatory domain, and CD3ζ signaling domain was generated as previously described (Milone M C, et al. Molecular therapy:the journal of the American Society of Gene Therapy. 2009; 17(8):1453-64). This is the same construct used in CTL019 trials at the University of Pennsylvania (Porter D L, et al. Blood. 2006; 107(4):1325-31).

### Cells and Cell Cultures

Peripheral blood leukocytes from healthy donors were obtained from the Human Immunology Core. Informed consent was obtained from all participants prior to collection. All methods and experimental procedures were approved by the University of Pennsylvania Institutional Review Board. T cells were purified at the University's Human Immunology Core by negative selection using the RosetteSep T cell enrichment Cocktail. T cells were serum starved in RPMI media containing 10 mM HEPES, 2 mM L-glutamine 100 U/mL penicillin G and 100 μg/mL streptomycin for 2-6 hours and then resuspended at a concentration of 10⁷ T cells/mL in X-VIVO 15 (Cambrex, Walkersville, Md.) supplemented with 5% normal human AB serum (Valley Biomedical, Winchester, Va.), 2 mM L-glutamine (Cambrex), 20 mM HEPES (Cambrex), IL7 and IL15 (10 ng/mL, Miltenyi Biotec), and deoxynucleosides (50 μM, Sigma). As the medium was switched to X-VIVO 15, T cells were simultaneously transduced with CD19-BBζ CAR or iRFP lentiviral supernatants for 24 hours.

In some assays, T cells were activated using stimulatory and culture conditions identical to the clinical test expansions for the CTL019 trials (Couzin-Frankel, J. Science 356, 1112-1113 (2017)). Briefly, fresh or cryopreserved donor cells were stimulated with magnetic beads precoated with agonist antibodies against CD3 and CD28 (Life Technologies) at a ratio of three beads per cell, and then resuspended at a concentration of 10⁶ T cells/mL for expansion in X-VIVO 15 supplemented with 5% normal human AB serum, 2 mM L-glutamine, 20 mM HEPES, and IL2 (100 units/mL; R&D Systems). T cells were then lentivirally transduced with CD19-BBζ CAR or iRFP at day 1 and expanded for 9 days or harvested at specific time points for analyses. Cells were maintained in culture at a concentration of 0.5×10⁶ cells/mL by adjusting the concentration every other day based on counting by flow cytometry using countbright beads (BD Bioscience) and monoclonal antibodies to human CD4 (clone OKT4) and CD8 (clone SK1) (O'Connor R S, et al., J Immunol. 2012; 189(3):1330-9). Cell volume was also measured with a Multisizer III particle counter (Beckman-Coulter) every other day.

### Flow Cytometry

1×10⁶ cells were stained for cell surface markers to analyze T-cell differentiation status. The following pre-titrated antibodies were used: anti-CCR7-FITC (clone 150503) (BD Pharmingen); anti-CD45RO-PE (clone UCHL1), anti-CD8-H7APC (clone SK1) (BD Biosciences); anti-CD4-BV510 (clone OKT4), anti-CD3-BV605 (clone OKT3), anti-CD14-Pacific Blue (PB) (clone HCD14), and anti-CD19-PB (clone HIB19) (BioLegend). The anti-CAR19 idiotype for surface expression of CAR19 was provided by Novartis (Basel, Switzerland). Cells were washed with phosphate-buffered saline (PBS) and stained for viability using LIVE/DEAD Fixable Violet (Molecular Probes) for 15 minutes, washed once, and resuspended in fluorescence activated cell sorting (FACS) buffer containing PBS, 1% BSA, and 5 mM EDTA. Cells were then incubated with the above indicated antibodies for 1 hour at 4° C. Samples were then washed three times with FACS buffer and fixed in 1% paraformaldehyde. Positively stained cells were differentiated from background using fluorescence-minus-one (FMO) controls. Flow cytometry was performed on BD LSR Fortessa. Analysis was performed using Flowjo software (Tree Star Inc. version 10.1).

### Quantitative (q) PCR Analysis

CAR T cells were harvested and genomic DNA was isolated. Using 200 ng genomic DNA, qPCR analysis was performed to detect the integrated BBζ CAR transgene sequence using ABI Taqman technology as described in (Kalos, M. et al. Science translational medicine 3 (2011); Guidance for Industry, Hum Gene Ther 12, 315-320 (2001)). To determine copy number per unit DNA, an 8-point standard curve was generated consisting of 5 to 10⁶ copies of the MK lentivirus plasmid spiked into 100 ng non-transduced control genomic DNA. The number of copies of plasmid present in the standard curve was verified using digital qPCR with the same CAR primer/probe set and performed on a QuantStudio™ 3D digital PCR instrument (Life Technologies). A CDKN1A-specific primer-probe set was used as a normalization control. Each datapoint (sample, standard curve) was evaluated in triplicate with a positive Ct value in 3/3 replicates with % CV<0.95% for all quantifiable values.

### In Vivo Models

A xenograft model was used as previously reported (Guidance for Industry, Hum Gene Ther 12, 315-320 (2001); Milone M C, et al., Molecular therapy: the journal of the American Society of Gene Therapy. 2009; 17(8):1453-64). Briefly, 6-10-week-old NOD-SCID γc−/− (NSG) mice, which lack an adaptive immune system, were obtained from Jackson laboratories (Bar Harbor) or bred in-house under a protocol approved by the Institutional Animal Care and Use Committees of the University of Pennsylvania. Animals were assigned in all experiments to treatment/control groups using a randomized approach. Animals were injected IV via tail vein with 2×10⁶ NALM6 cells (ATCC) in 0.1 mL sterile PBS. CART19 cells or non-transduced (UTD) human T cells were injected via tail vein at the indicated dose in a volume of 100 μL of sterile PBS/Ca²⁺ 4 days after injection of NALM6. Anesthetized mice were imaged using a Xenogen IVIS Spectrum system (Caliper Life Science) twice a week. Mice were given an intraperitoneal injection of D-luciferin (150 mg/kg; Caliper Life Sciences). Total flux was quantified using Living Image 4.4 (PerkinElmer) by drawing rectangles of identical area around mice, reaching from head to 50% of the tail length. Background bioluminescence was subtracted for each image individually. Peripheral blood was obtained by retro-orbital bleeding in an EDTA coated tube, and blood was examined immediately for evidence of T cell engraftment by flow cytometry using BD Trucount (BD Biosciences). Animals were euthanized at the end of the experiment or when they met pre-specified endpoints according to the IACUC protocols (before reaching signals higher than 1×10¹¹ p/s total flux per mouse, or before the disease was too well established to reverse with therapy).

### Statistical Analysis

The graphs represent the mean value+standard deviation (SD), unless otherwise indicated. A student's t test for paired data, Wilcoxon rank-sum test, or a one-way ANOVA were performed using GraphPad Prism version 4.0a (GraphPad Software). Multiple-comparison post-hoc corrections were performed using the Neuman-Keuls test. A p-value <0.05 was considered statistically significant.

### Example 2: Enhancing the Therapeutic Index of CAR T Cell Therapy

Chimeric antigen receptor (CAR) T cells are potent therapies for cancer. The success of CAR T cell therapy in various hematopoietic malignancies including B cell acute lymphoblastic leukemia (ALL), chronic lymphocytic leukemia (CLL) and multiple myeloma highlights the therapeutic promise of this approach. The recent FDA approvals for two CD19-specific CAR T cell therapies, tisagenlecleucel for pediatric and young adult ALL and axicabtagene ciloleucel for diffuse large B-cell lymphoma, reinforces the translational merit of CAR T Therapy. Despite the high overall rate of complete response to CD19-specific CAR therapy in ALL, some patients still relapse as CAR-T cells undergo senescence (Maude, S. L. et al. The New England journal of medicine 371, 1507-1517 (2014)).

The efficacy of CAR T cell immunotherapy relies on the quality of manufactured CAR T cells. The success of redirected CAR T depends on the ability of the infused T cells to engraft, expand and persist, providing long term immunosurveillance following adoptive transfer (Grupp, S. A. et al. The New England journal of medicine 368, 1509-1518 (2013); Porter, D. L. et al. Science translational medicine 7, 303ra139 (2015); Maude, S. L., Blood 125, 4017-4023 (2015); Kochenderfer, J. N. et al. J Clin Oncol 35, 1803-1813 (2017)). T cells consist of several distinct subsets including: naïve T cells (Tn), central memory (Tcm), effector memory (Tem), effector differentiated (Tte) and stem cell memory (Tscm). Effector differentiated T cells have potent cytolytic ability, however, they are short lived and engraft poorly (Bollard, C. M., et al. Nature reviews. Clinical oncology 9, 510-519 (2012); Brestrich, G. et al. Am J Transplant 9, 1679-1684 (2009); Savoldo, B. et al. Blood 108, 2942-2949 (2006)). In contrast, T cells with a less-differentiated phenotype including naïve T cells and Tcm exhibit superior engraftment and proliferative abilities following adoptive cell transfer (Berger, C. et al. The Journal of clinical investigation 118, 294-305 (2008); Gattinoni, L. et al. Nat Med 17, 1290-1297 (2011); Hinrichs, C. S. et al. Proceedings of the National Academy of Sciences of the United States of America 106, 17469-17474 (2009); Klebanoff, C. A. et al. Proceedings of the National Academy of Sciences of the United States of America 102, 9571-9576 (2005); Wang, X. et al. Blood 117, 1888-1898 (2011); Wang, X. et al. Oncoimmunology 5, e1072671 (2016)). Recent correlative data from clinical trials of tisagenlecleucel at the University of Pennsylvania that show the proportion of T cells with a naïve-like immunophenotype in the starting apheresis product is highly correlated with both engraftment and clinical response (Fraietta J, et al., Determinants of Response and Resistance to CD19 Chimeric Antigen Receptor (CAR) T cell Therapy of Chronic Lymphocytic Leukemia. Nature Medicine In press 2018). Taking together, these findings show that cultured T cells derived from less differentiated precursors give rise to superior engraftment and persistence following adoptive transfer in both tumor and infectious disease models (Berger, C. et al. The Journal of clinical investigation 118, 294-305 (2008); Gattinoni, L. et al. Nat Med 17, 1290-1297 (2011); Hinrichs, C. S. et al. Proceedings of the National Academy of Sciences of the United States of America 106, 17469-17474 (2009); Graef, P. et al. Immunity 41, 116-126 (2014); Hinrichs, C. S. et al. Blood 117, 808-814 (2011)) and human patients (Oliveira, G. et al. Science translational medicine 7, 317ra198 (2015)).

T cells expansion ex vivo leads to progressive differentiation. CAR T-based approaches almost universally involve the isolation of T cells from peripheral blood, activation, genetic modification, and expansion of patient T cells ex vivo. Beads coated with agonist antibodies to the CD3 complex and the CD28 costimulatory receptor, are commonly used to activate T cells. Following activation, T cells enter a phase of rapid proliferation that is associated with progressive differentiation. A number of approaches have been developed to limit activation-induced differentiation including inhibition of Fas-FasL interactions (Klebanoff, C. A. et al. J Clin Invest 126, 318-334 (2016)), inhibition of Akt signaling (Crompton, J. G. et al. Cancer Res 75, 296-305 (2015); van der Waart, A. B. et al. Blood 124, 3490-3500 (2014)) or alternatively activating Wnt signaling (Gattinoni, L. et al. Nat Med 17, 1290-1297 (2011); Gattinoni, L. et al. Nature medicine 15, 808-813 (2009); Muralidharan, S. et al. The Journal of Immunology 187, 5221-5232 (2011)). While these approaches may limit differentiation, eliminating the stimulation and ex vivo culture of T cells offer a far simpler and more cost-effective approach. Effective transduction of naïve T cells without inducing differentiation could markedly enhance engraftment as naïve T cells have been shown to have a very long intermitotic half-life, with estimates for humans of approximately 3.5 years (McLean, A. R. & Michie, C. A. Proceedings of the National Academy of Sciences of the United States of America 92, 3707-3711 (1995); Tough, D. F. & Sprent, J. The Journal of experimental medicine 179, 1127-1135 (1994)). Such cell cycle parameters are an important determinant underlying effective hematopoietic stem cell engraftment.

The ability to generate CAR T cells with less ex vivo culture has important practical implications for CAR T manufacturing. Ultra-short culture durations will lead to a shorter period of time between apheresis collection and reinfusion of T cell products. This has important implications for treating patients, especially those with rapidly progressive disease (Couzin-Frankel, J. Science 356, 1112-1113 (2017)). Extended culture durations are costly. In addition to reduced labor, a limited ex vivo culture process would conserve resources such as human serum and scarce manufacturing space. Lentiviral vectors are capable of transducing non-dividing cells. It is well established that natural HIV has the ability to infect quiescent T cells in the G0 stage of the cell cycle (Naldini, L. et al. Science 272, 263-267 (1996); Plesa, G. et al. J Virol 81, 13938-13942 (2007); Swiggard, W. J. et al. J Virol 79, 14179-14188 (2005)). Unlike gamma-retroviruses, HIV-derived lentiviral vectors are capable of transducing both dividing and non-dividing cells. However, transduction efficiency of quiescent T cells has been generally quite low compared with activated T cells (Korin, Y. D. & Zack, J. A. J Virol 73, 6526-6532 (1999); Rausell, A. et al. Retrovirology 13, 43 (2016)).

Without wishing to be bound by theory, CAR T cells generated by transducing quiescent T cells will preserve the intrinsic stem-like properties of naïve and memory T cells. This approach yields CAR T cells with enhanced replicative capacity, engraftment and in vivo activity.

### Example 3: Ultra-Short Manufacturing of Quiescent Chimeric Antigen Receptor T Cells for Adoptive Immunotherapy

The recent success of immunotherapy using chimeric antigen receptor modified T cells (CART) in ALL highlights the potential of these cytotoxic “drugs” for cancer therapy. All current CART therapies rely on the prior activation, genetic modification and expansion of patient-derived T cells. 6-14 days of ex vivo culture are routinely used to generate large numbers of cells for adoptive transfer. However, the commonly used CD3/CD28 stimulation, while a potent mitogenic signal for T cells, promotes progressive T cell effector differentiation over time in culture. This differentiation is associated with reduced engraftment and reduced long-term persistence of T cells required for durable anti-tumor efficacy. Since cell division is not a prerequisite for lentiviral vector-mediated gene delivery, it was hypothesized that lentiviral transduction of quiescent T cells without prior activation will enhance engraftment and persistence of CART cells that is associated with long-term leukemia control.

Described here is a novel approach that can generate potent CD19-specific CART cells using lentiviral vectors that can be infused within 24 hours of T cell collection. In a murine xenograft model of acute lymphoblastic leukemia, CD19-specific CART cells generated using this ultrashort manufacturing process exhibit potent, dose-dependent anti-leukemic activity associated with persistent engraftment and durable anti-leukemic activity (FIGS. 1D and 2C). CART cells manufactured using this highly abbreviated process also exhibit a greater fraction of naïve-like and central memory T cells when compared with standard anti-CD3/CD28 microbead-based manufacturing.

The ultrashort manufacturing approach described has the potential to markedly reduce the ex vivo manipulations required for CART cell manufacturing, providing a fast, simple and less costly method for achieving a potent CART cell immunotherapy.

### Example 4: Rapid Manufacturing of Chimeric Antigen Receptor (CAR) T Cells without T Cell Activation

### Abstract

Chimeric antigen receptor (CAR) T cell therapies are able to generate deep and durable clinical responses in hematologic malignancies of the B-cell lineage. The manufacturing of these T cell-based therapies typically relies upon viral transduction of T cell-receptor (TCR) activated T cell followed by ex vivo expansion for 6 or more days prior to infusion. In addition to the required time and labor, the TCR/CD3 activation and ex vivo expansion leads to progressive differentiation of the CAR T cells with associated loss of anti-leukemic activity. This example shows that functional CAR T cells can be generated within less than 24 hours from peripheral blood-derived T cells without the need for prior T cell activation in a process that is significantly influenced by the medium formulation and geometry of the transduction vessel. Using a CD19-specific CAR, T cells generated using this simple and rapid manufacturing approach exhibited anti-leukemic activity in vitro. In a well-established xenograft model of B-cell acute lymphoblastic leukemia, these non-activated and rapidly produced T cells engrafted and showed durable control of leukemia despite a lower transduced T cell dose when compared to T cells that have been activated and expanded ex-vivo by a process used in the development of tisagenlecleucel. These data illustrate the potential for significantly reducing the time and cost of CAR T cell production required to expand the application of this therapy to patients with rapidly progressive disease as well as resource-poor healthcare settings.

### Introduction

Adoptive cellular immunotherapy (ACT) using T cells that are genetically modified to express a chimeric antigen receptor (CAR) or cloned T cell receptor (TCR) yield durable clinical responses in patients with cancer (Salter et al., Blood. 2018; 131(24):2621-2629; Brudno et al., J Clin Oncol. 2018; 36(22):2267-2280; Cohen et al., J Clin Invest. 2019; 129(6):2210-2221; D'Angelo et al., Cancer Discov. 2018; 8(8):944-957; Raje N, et al., N Engl J Med. 2019; 380(18):1726-1737; Kalos et al., Sci Transl Med. 2011; 3(95):95ra73). The effectiveness of ACT led to the regulatory approval of two CD19-specific CAR T cell therapies, tisagenlecleucel and axicabtagene ciloleucel. Both of these therapies involve the isolation of mononuclear cells containing T cells from a patient's peripheral blood, followed by T cell activation through their endogenous TCR/CD3 complex, genetic modification using a viral vector and expansion ex vivo before reinfusion. It was recently shown that activated T cells undergoing rapid proliferation ex vivo differentiate toward effector cells with loss of anti-leukemic potency (Ghassemi et al., Cancer Immunol Res. 2018; 6(9):1100-1109). The ability of T cells to engraft following adoptive transfer is related to their state of differentiation with less differentiated naïve-like and central memory cells showing the greatest potency in several preclinical studies (Berger et al., J Clin Invest. 2008; 118(1):294-305; Graef P, et al., Immunity. 2014; 41(1):116-126; Hinrichs et al., Proc Natl Acad Sci USA. 2009; 106(41):17469-17474; Hinrichs et al., Blood. 2011; 117(3):808-814). A number of interventions have been reported to limit the differentiation of T cells and enhance the potency of ex vivo expanded T cells such as blockade of Fas-FasL interactions (Klebanoff et al., J Clin Invest. 2016; 126(1):318-334), inhibition of Akt signaling (Crompton et al., Cancer Res. 2015; 75(2):296-305; van der Waart et al., Blood. 2014; 124(23):3490-3500) or activation of Wnt signaling (Gattinoni et al., Nature medicine. 2009; 15(7):808-813; Gattinoni et al., Nat Med. 2011; 17(10):1290-1297; Muralidharan et al., The Journal of Immunology. 2011; 187(10):5221-5232). However, eliminating the activation step and corresponding proliferative phase of T cells ex vivo offers a far simpler and more cost-effective approach provided the barriers to gene transfer into quiescent T cells can be overcome.

Natural HIV has the ability to infect quiescent T cells in the G0 stage of the cell cycle (Naldini et al., Science. 1996; 272(5259):263-267; Plesa et al., J Virol. 2007; 81(24):13938-13942; Swiggard et al., J Virol. 2005; 79(22):14179-14188). Unlike gamma-retroviruses, HIV-based lentiviral vectors can infect both dividing and non-dividing cells. However, the transduction efficiencies in quiescent T cells are typically lower than their activated counterparts. Lentiviral infection is a multi-step process involving binding of the viral particle to the T cell plasma membrane and endocytosis followed by envelope fusion, reverse transcription (RT) to form a pre-integrated provirus and finally integration into the host T cell genome. Lentiviral particles that are pseudotyped with a vesicular stomatitis virus g-glycoprotein (VSV-G) to broaden the viral tropism depend upon the low-density lipoprotein (LDL) receptor which is ubiquitously expressed on the surface of various cells including lymphocytes for entry (Amirache et al., Blood. 2014; 123(9):1422-1424; Finkelshtein et al., Proc Natl Acad Sci USA. 2013; 110(18):7306-7311). Limitations to efficient lentiviral transduction of quiescent T cells have been identified at each step. Lentiviral particles fuse inefficiently with quiescent T cells (Agosto et al., J Virol. 2009; 83(16):8153-8162; Pace et al., J Virol. 2011; 85(1):644-648). Conditioning the cell culture medium with recombinant cytokines IL-7 and IL-15 can overcome this limitation (Unutmaz et al., J Exp Med. 1999; 189(11):1735-1746) and increase transduction efficiencies as well as cell survival in quiescent T cells (Cavalieri et al., Blood. 2003; 102(2):497-505; Trinite et al., J Virol. 2016; 90(2):904-916). Engineering viral particles to express an IL-7 fusion protein increased quiescent T cell lentiviral transduction (Verhoeyen et al., Blood. 2003; 101(6):2167-2174). Post-entry, low concentrations of nucleotides and the presence of additional restriction factors such as SAMHD1 limit the rate of reverse transcription in quiescent T cells (Korin et al., J Virol. 1999; 73(8):6526-6532; Rausell et al., Retrovirology. 2016; 13(1):43; Descours et al., Retrovirology. 2012; 9:87). Collectively, these factors make lentiviral transduction of non-activated T cell inefficient.

Having previously shown that shortening the ex vivo culture of CAR T cells yields a cellular product with less differentiated T cells and significantly enhanced effector function, we hypothesized that elimination of the CD3/CD28 activation step, if effective gene transfer into T cells could be achieved, would yield a T cell product with high functional potency. Reducing the culture duration could also substantially reduce the vein-to-vein time, expensive medium and labor required to make CAR T cell products. This example shows that non-activated T cells can be effectively transduced with a CAR by lentiviral vector. This example further demonstrates that some of the restriction factors that limit the infection of quiescent T cells can be overcome by changes to the medium formulation. Most importantly, this example shows that non-activated T cells exhibit potent antitumor function with deep and durable control of leukemia achieved in vivo with greater than 1-log fewer CD19-specific CAR T cells produced within 24 hours of mononuclear cell collection compared with a 9-day process currently used by tisagenlecleucel. These results demonstrate the potential for vastly reducing the time, materials and labor required to generate CAR T cells, which could be especially beneficial in patients with rapidly progressive disease and in resource-poor health care environments.

### Methods

### Generation of Lentiviral Vectors

Replication defective lentivirus was produced by standard methods using a 3rd generation lentiviral vector transfer plasmid encoding infrared fluorescent protein (iRFP) or a CD19-BBζ CAR (Milone et al., Mol Ther. 2009; 17(8):1453-1464) mixed with three packaging plasmids encoding VSVg (pMDG.1), gag/pol (pMDLg/pRRE) and rev (pRSV-rev), and transfected into HEK293T cells using Lipofectamine 2000 (Invitrogen).

### Cells

Peripheral blood leukocytes from healthy donors were obtained from the Human Immunology Core. Informed consent was obtained from all participants prior to collection. All methods and experimental procedures were approved by the University of Pennsylvania Institutional Review Board. T cells were purified at the University's Human Immunology Core by negative selection using the RosetteSep T cell enrichment Cocktail.

All cell lines (NALM-6 and HEK293T cell) were originally obtained from the American Type Culture Collection (ATCC). Cells were expanded in RPMI medium containing 10% fetal bovine serum (FBS), penicillin and Streptomycin at a low passage and tested for mycoplasma.

### Transduction of T Cells

For activated T cells, cells were resuspended at 10⁶ T cells/mL in X-VIVO 15 (Cambrex, Walkersville, Md.) supplemented with 5% human AB serum (Valley Biomedical, Winchester, Va.), 2 mM L-glutamine (Cambrex), 20 mM HEPES (Cambrex), IL-7 and IL-15 (10 ng/mL, Miltenyi Biotec). Dynabeads™ Human T-Activator CD3/CD28 beads (Thermofisher) were added to a final ratio of 3 beads to 1 cell. After 24 hours, lentiviral vector supernatant was added at a multiplicity of infection (MOI) as indicated. Cells were maintained in culture at a concentration of 0.5×10⁶ cells/mL by adjusting the concentration every other day based on counting by flow cytometry using countbright beads (BD Bioscience) and monoclonal antibodies to human CD4 and CD8 (O'Connor et al., J Immunol. 2012; 189(3):1330-1339). Cell volume was also measured with a Multisizer III particle counter (Beckman-Coulter) every other day. For non-activated T cells, cells were resuspended at 10⁷ T cells/mL in X-VIVO 15 supplemented with 2 mM L-glutamine, 20 mM HEPES, IL-7 and IL-15 (10 ng/mL) for 3-6 hours followed by addition of human AB serum to 5% of the total volume and lentiviral vector supernatant to achieve an MOI as indicated. Deoxynucleosides (50 μM) were also added to the medium in some experiments as indicated.

### Flow Cytometry

T cell differentiation was assessed using the following antibodies: anti-CCR7-FITC (BD Pharmingen); anti-CD45RO-PE, anti-CD8-H7APC (BD Biosciences); anti-CD4-BV510, anti-CD3-BV605, anti-CD14-Pacific Blue (PB), anti-CD19-PB (BioLegend). The anti-CAR19 idiotype for surface expression of CAR19 was provided by Novartis (Basel, Switzerland). Cells were washed with phosphate-buffered saline (PBS), incubated with LIVE/DEAD Fixable Violet (Molecular Probes) for 15 minutes, and resuspended in fluorescence activated cell sorting (FACS) buffer consisting of PBS, 1% BSA, and 5 mM EDTA. Cells were then incubated with antibodies for 1 hour at 4° C. Positively stained cells were differentiated from background using fluorescence-minus-one (FMO) controls. Flow cytometry was performed on BD LSR Fortessa. Analysis was performed using Flowjo software (Tree Star Inc. version 10.1).

### Quantitative (q) PCR Analysis

Genomic DNA was isolated using a QlAamp DNA Micro Kit (Qiagen). Using 200 ng genomic DNA, qPCR analysis was performed to detect the integrated MK CAR transgene sequence using ABI Taqman technology as previously described (Milone et al., Mol Ther. 2009; 17(8):1453-1464; Kalos et al., Science Translational Medicine. 2011; 3(95)). To determine copy number per μg of genomic DNA, an 8-point standard curve was generated consisting of 5 to 10⁶ copies of the BBt lentivirus plasmid spiked into 100 ng non-transduced control genomic DNA. A primer-probe set specific for the CDKN1A gene, a single copy gene in the human haploid genome, was used as a normalization control to estimate vector copies per cell.

### Cytokine Secretion

T cells were incubated at a ratio of 1:1 with irradiated target cells at a concentration of 10⁶ T cells/mL in a cytokine free media. Supernatants were collected at 24 hours to assess cytokine production. Measurement of Cytokine was performed with a Luminex bead array platform (Life Technologies) according to the manufacturer's instructions (Maude et al., N Engl J Med. 2014; 371(16):1507-1517).

### Cytotoxicity Assays

Cytotoxicity assays were performed using a ⁵¹Cr release-assay as previously described (Ghas semi et al., Cancer Immunol Res. 2018; 6(9):1100-1109). In brief, Na2⁵¹CrO4-labeled target cells were incubated with CART cells for 20 hours at various effector:target ratios. Chromium release into the supernatant was measured with a liquid scintillation counter (MicroB eta trilux, Perkin Elmer).

### In Vivo Models

A xenograft model was used as previously reported (Milone M C, et al., Mol Ther. 2009; 17(8):1453-1464). Animals were assigned in all experiments to treatment/control groups using a randomized approach. Animals were injected IV via tail vein with 2×10⁶ NALM6 cells in 0.1 mL sterile PBS. CART19 cells or non-transduced (UTD) human T cells were injected via tail vein at the indicated dose in a volume of 100 μL, 4 days after injection of NALM6. Anesthetized mice were imaged using a Xenogen IVIS Spectrum system (Caliper Life Science). Total flux was quantified using Living Image 4.4 (PerkinElmer). T cell engraftment was defined as >1% human CD45+ cells in Peripheral blood by flow cytometry. Animals were euthanized at the end of the experiment or when they met pre-specified endpoints according to the protocols.

### Results

### Transduction of Non-Activated T Cells by Lentiviral Vectors

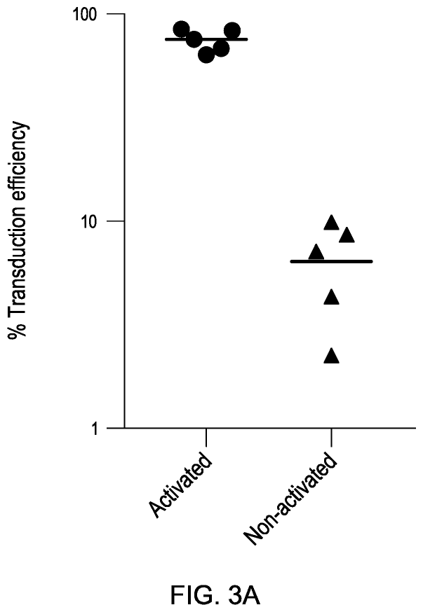

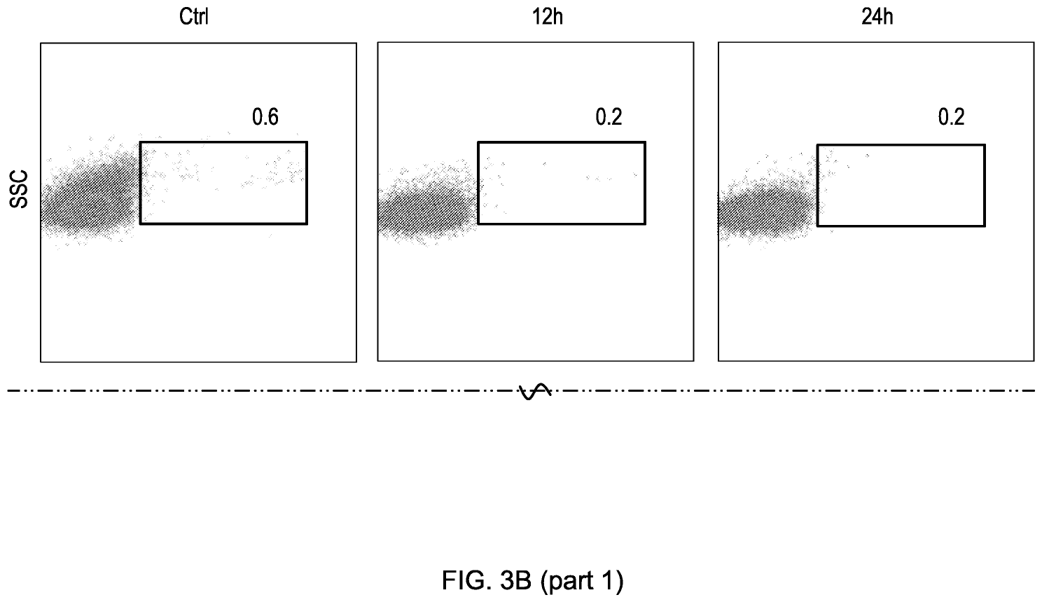

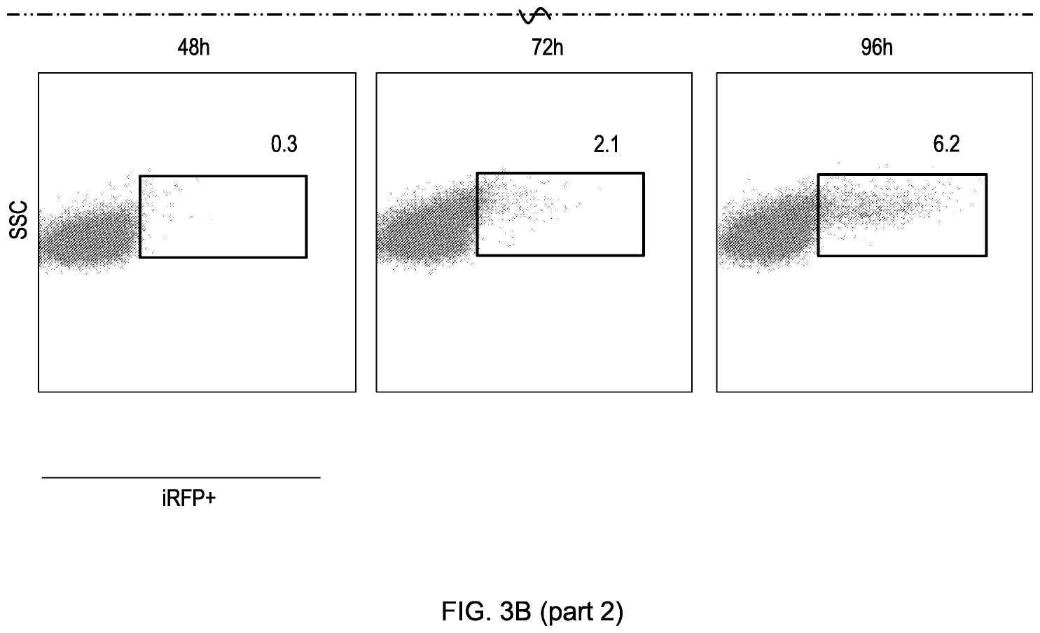

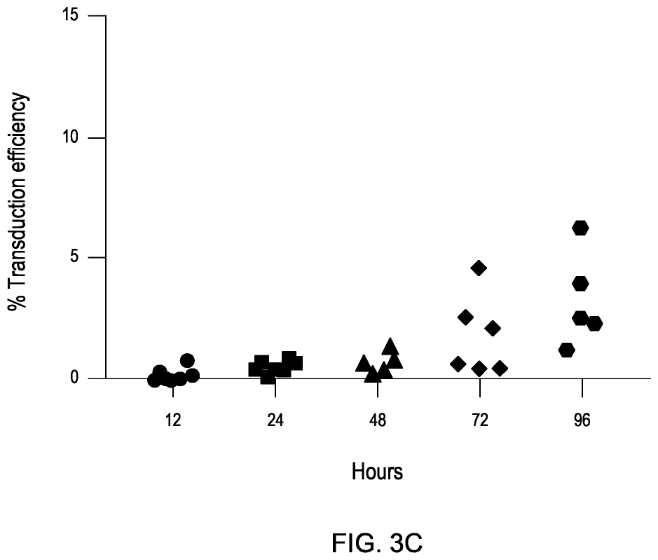

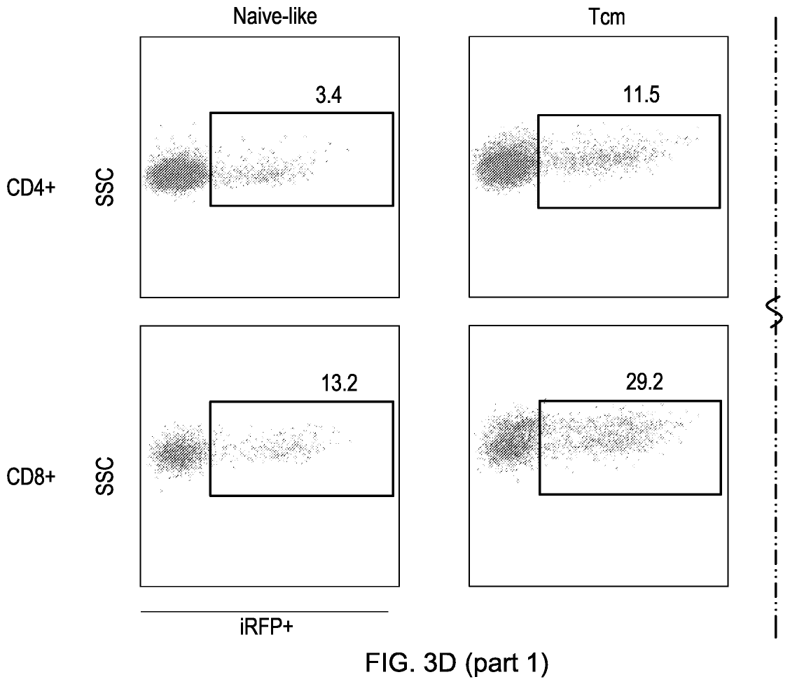

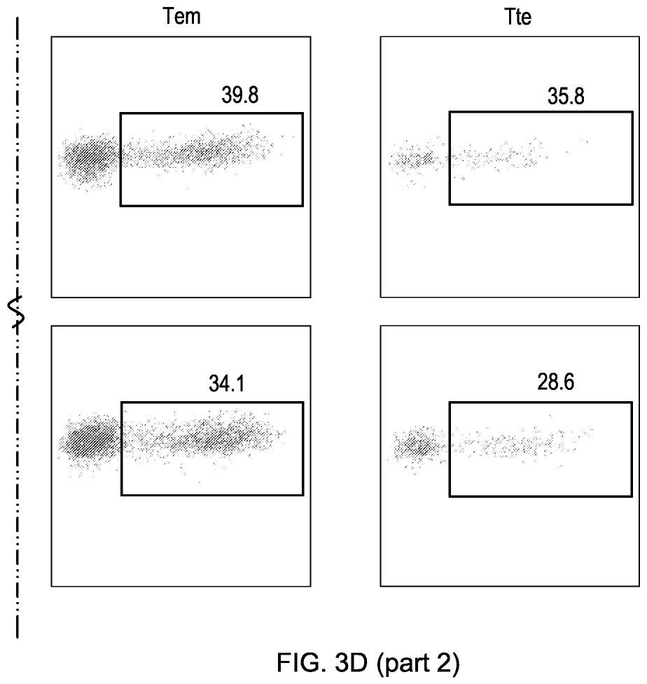

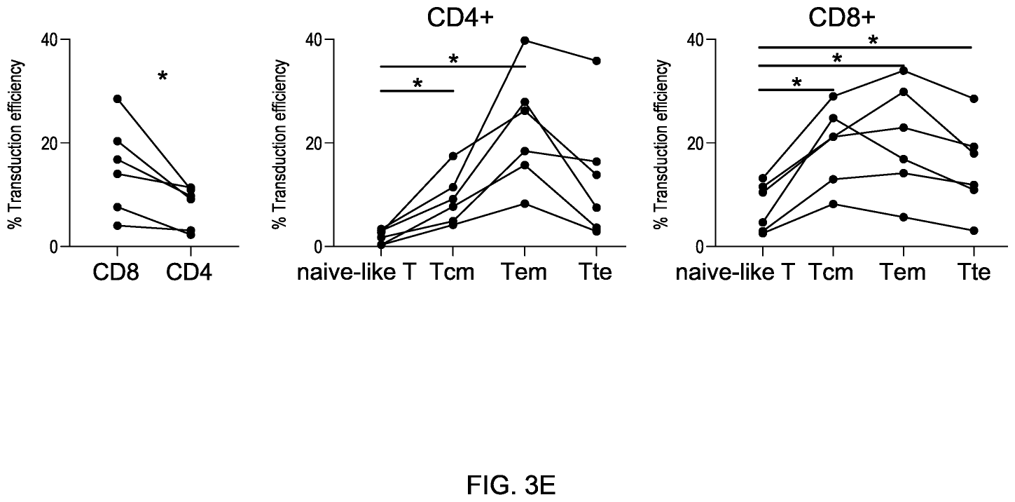

The previously reported, low transduction efficiency of freshly isolated, quiescent T cells was confirmed. Primary human T cells obtained from healthy donors were mixed with an infrared red fluorescent protein (iRFP)-encoding lentiviral vector under conditions identical to activated T cells, and followed for 96-hours to assess the efficiency and kinetics of transduction (FIG. 3A). In comparison to lentiviral transduction of T cells activated 24 hours prior with anti-CD3/28 microbeads at a multiplicity of infection (MOI) of 5 that yielded >85% transduction at 48 hours, the efficiency (FIG. 3A) and kinetics (FIG. 3B) of lentiviral transduction in non-activated T cells was substantially slower, requiring at least 72 hours to achieve detectable expression of an iRFP transgene with a transduction efficiency at 96 hours that was about 11 folds lower than activated T cells (FIGS. 3B-3C). The slower kinetics of this process is consistent with the decreased rate of reverse transcription reported for natural HIV in quiescent T cells compared with activated T cells (Pierson et al., J Virol. 2002; 76(17):8518-8531). Transduction was observed in both memory and naïve subsets of CD4+ and CD8+ T cells with the highest efficiency in CD8+ cells with a memory phenotype (FIGS. 3D-3E). Quiescent memory T cells appear more susceptible to lentiviral transduction than their naïve counterparts (FIGS. 3D-3E).

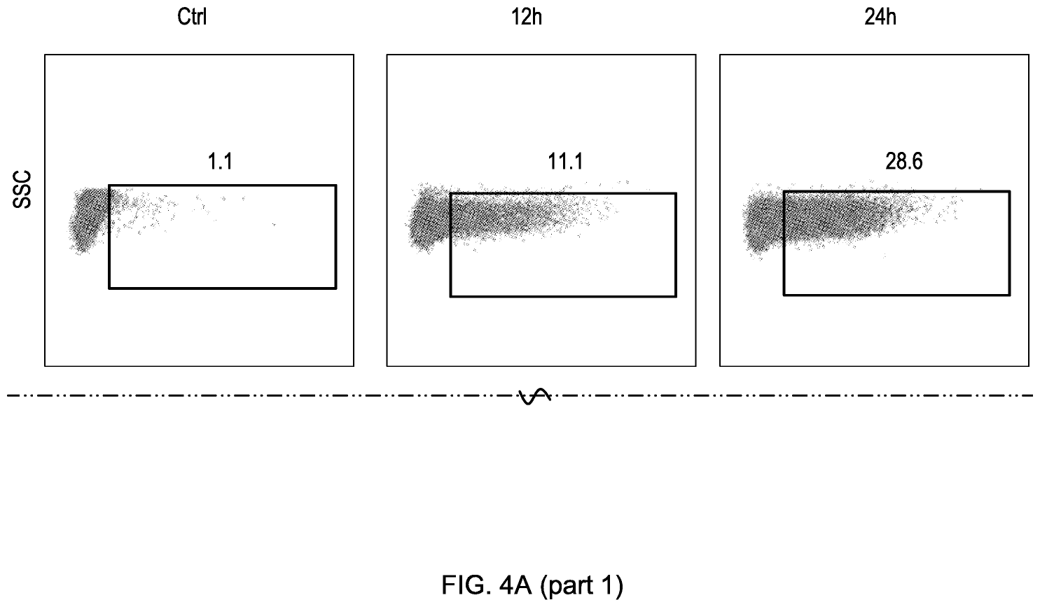

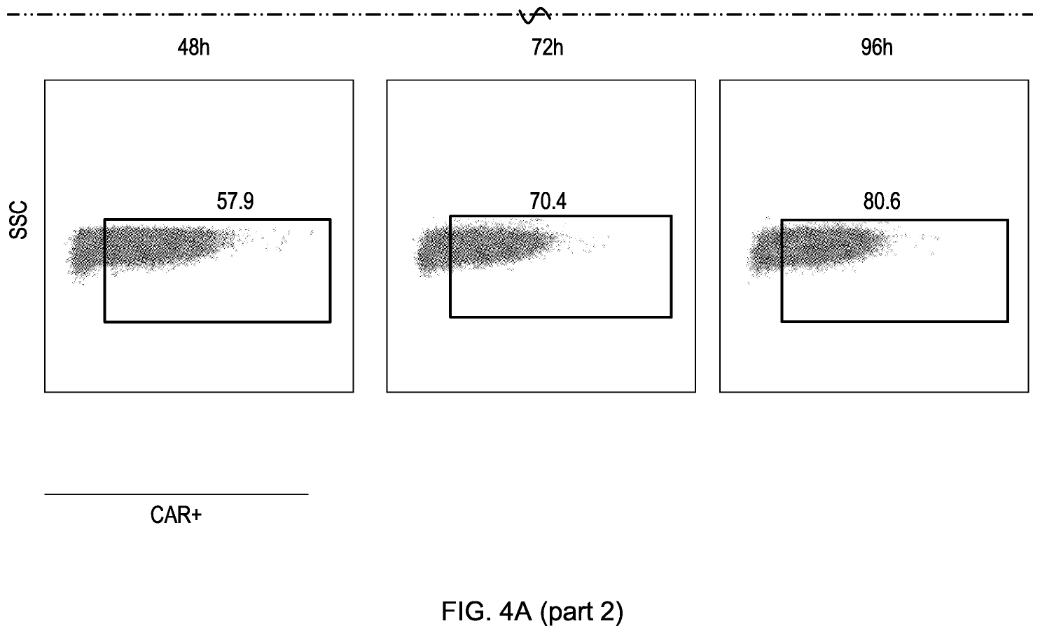

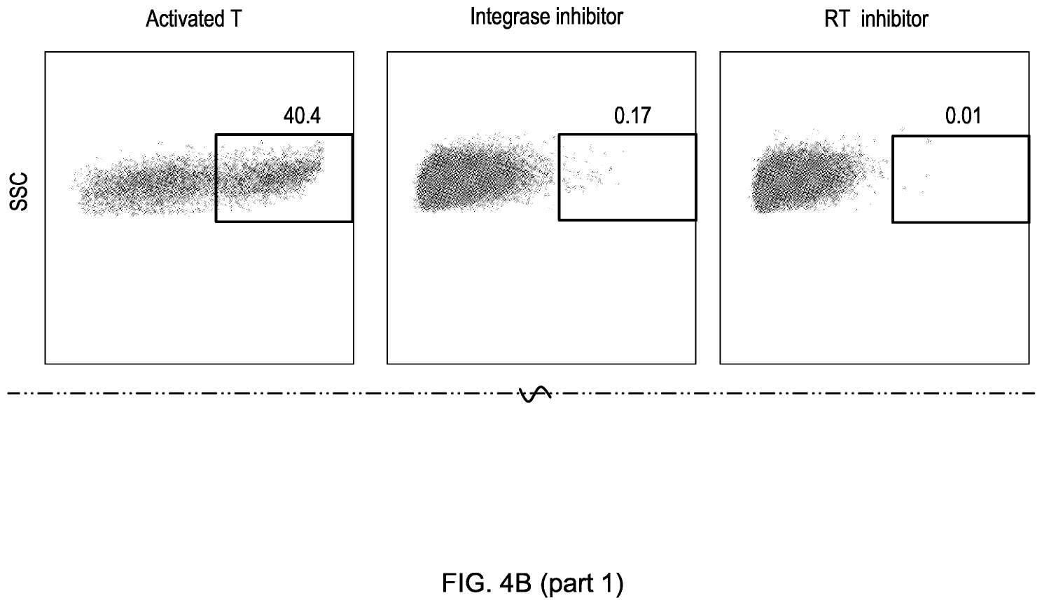

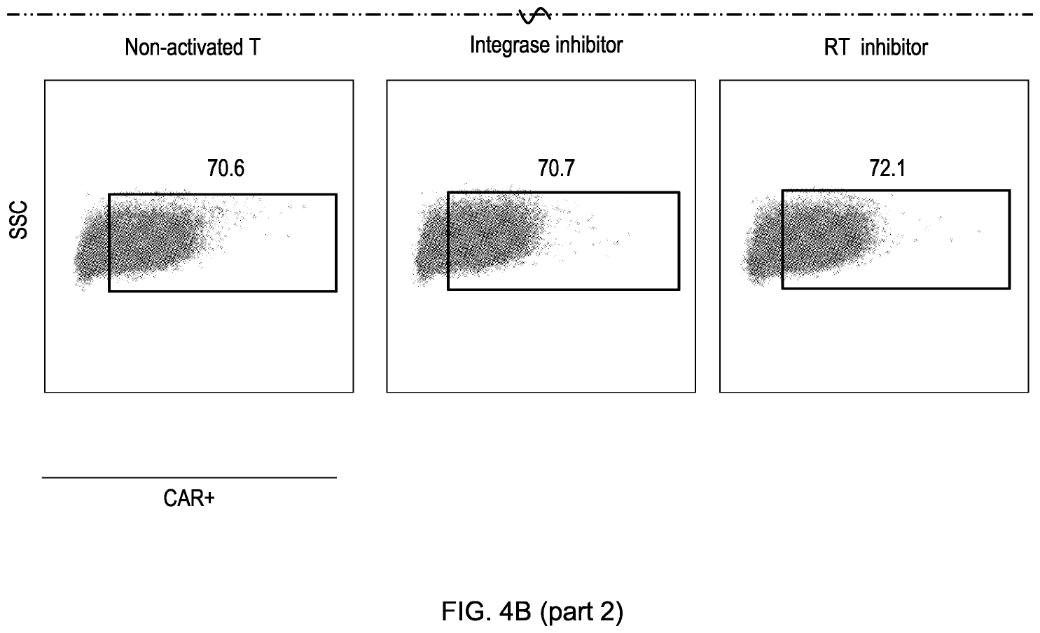

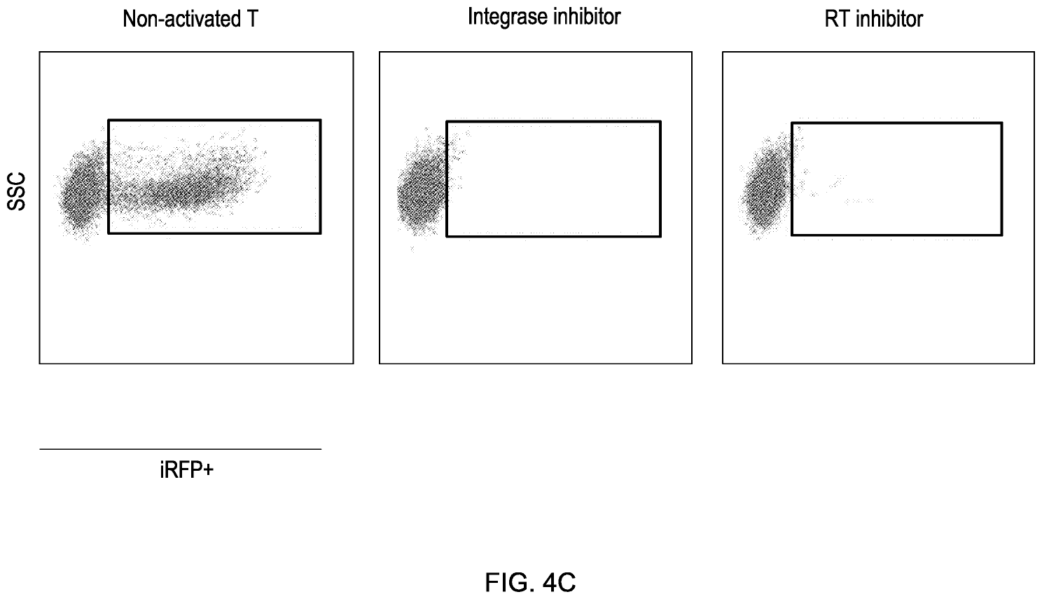

To extend these studies to a CAR, the above studies were repeated using a 3rd generation lentiviral vector encoding a CD19-specific CAR (CAR19). CAR expression was measured by immunostaining with a monoclonal antibody recognizing the idiotype of the single chain variable fragment (Milone et al., Mol Ther. 2009; 17(8):1453-1464). In a similar kinetic analysis to that performed with the iRFP-encoding lentiviral vector, it was observed that non-activated T cells initiated CAR expression as early as 12 hours with a steady increase to >80% of T cells by 96 hours (FIG. 4A). To determine if CARs were stably expressed, the T cells were treated with either a reverse transcriptase (RT) or integrase inhibitor during the lentiviral transduction process. As shown in FIG. 4B, CAR expression was unaffected by either compound in non-activated T cells in contrast with activated T cells where both RT or integrase inhibition completely abrogated CAR expression. This pseudotransduction observed in non-activated T cells is likely due to transfer of CAR protein from the lentiviral vector envelope to the T cell as membrane proteins expressed in the packaging cells are well known to incorporate into HIV's envelope (Burnie et al., Viruses. 2019; 11(1)). The absence of appreciable pseudotransduction with a vector that encodes iRFP, a cytoplasmic protein, supports this envelope-mediated transfer mechanism (FIG. 4C). Importantly, CD19-specific CAR T (CART19) cells generated by lentiviral transduction of non-activated T cells in the presence of RT and integrase inhibitors showed no specific cytolytic activity or cytokine production against CD19-expressing target cells (data not shown). Based on these data, it was concluded that long term persistence of CAR expression in T cells requires vector integration, which occurs at a substantially lower frequency in non-activated T cells compared with activated T cells.

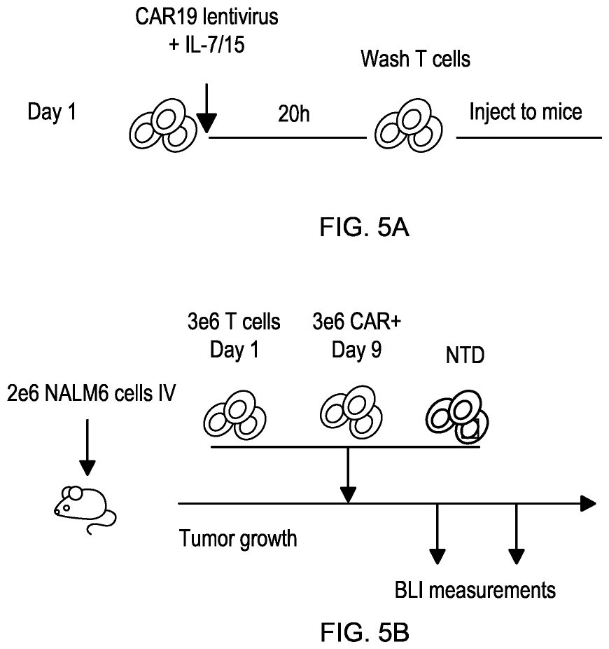

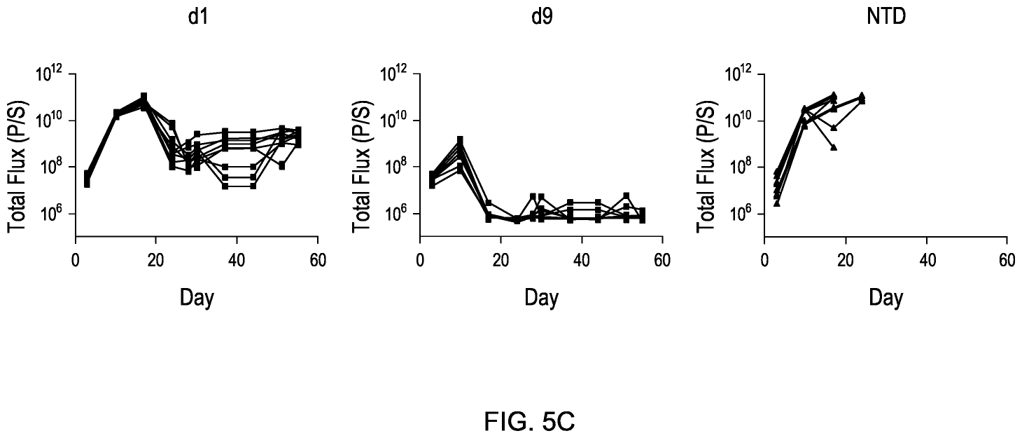

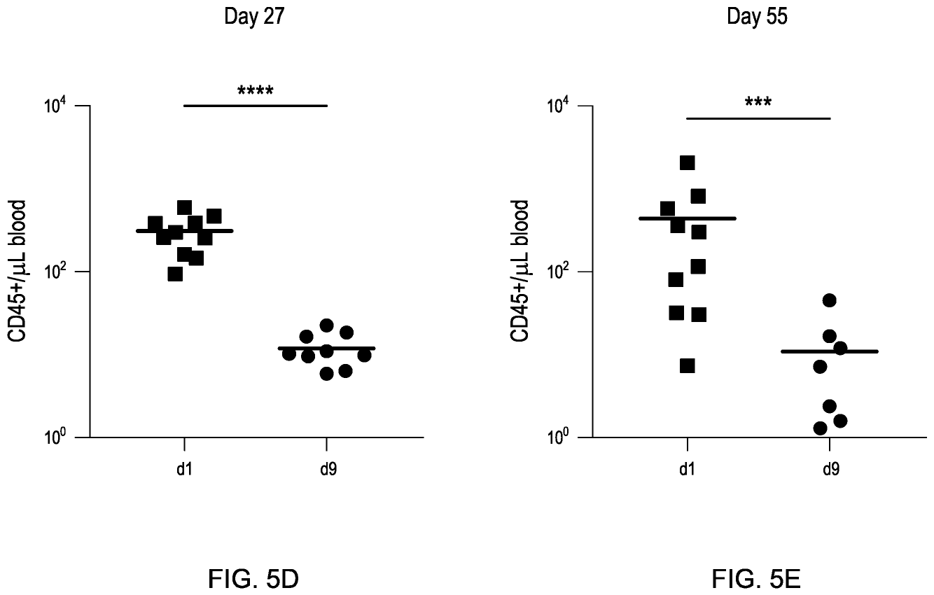

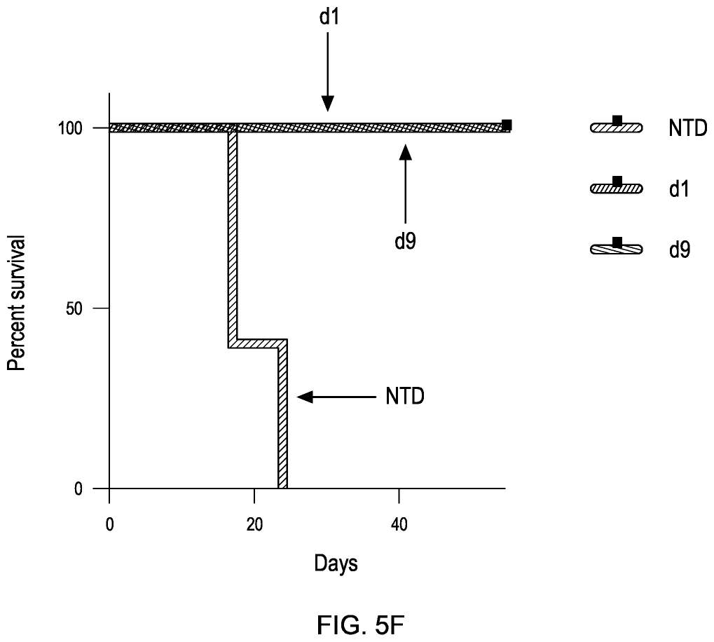

Although functional CAR expression was non assessible post-transduction without prolonged culture in IL-7 and IL-15 to permit completion of the viral RT and integration process, it was hypothesized that this process would likely complete itself following adoptive transfer in vivo giving rise to functional CART19 cells. An in vivo experiment was performed to evaluate non-activated CART19 cells transduced for 24 hours as in FIGS. 3A-3E using the well-established Nalm6 B-cell acute lymphoblastic leukemia model similar to that used to evaluate the potency of CAR T cells generated by vector-free gene editing approaches (FIGS. 5A-5F) (Eyquem et al., Nature. 2017; 543(7643):113-117). Day 9 activated CART19 cells prepared using a process comparable to that used to generate tisagenleculeucel was used as a control. As shown in FIGS. 5A-5F, a dose of 3×10⁶ non-activated CART19 cells induced regression of advanced Nalm6 disease, albeit with a substantial delay compared with activated CART19 cells prepared with the standard 9-day manufacturing process. The non-activated T cells were able to control leukemia for a prolonged period of time suggesting that the non-activated CART19 cells have considerable replicative capacity (FIG. 5C). The control of leukemia with non-activated T cells was consistent with the persistence of T cells in the peripheral blood of mice (FIGS. 5D-5E) and contributed to a significant increase in overall survival of the leukemia-bearing mice (FIG. 5F). These results support the functional nature of non-activated CART19 cells, and encouraged the optimization of the transduction process to further enhance their activity.

### Modifying the Culture Conditions to Enhance Non-Activated T Cell Transduction

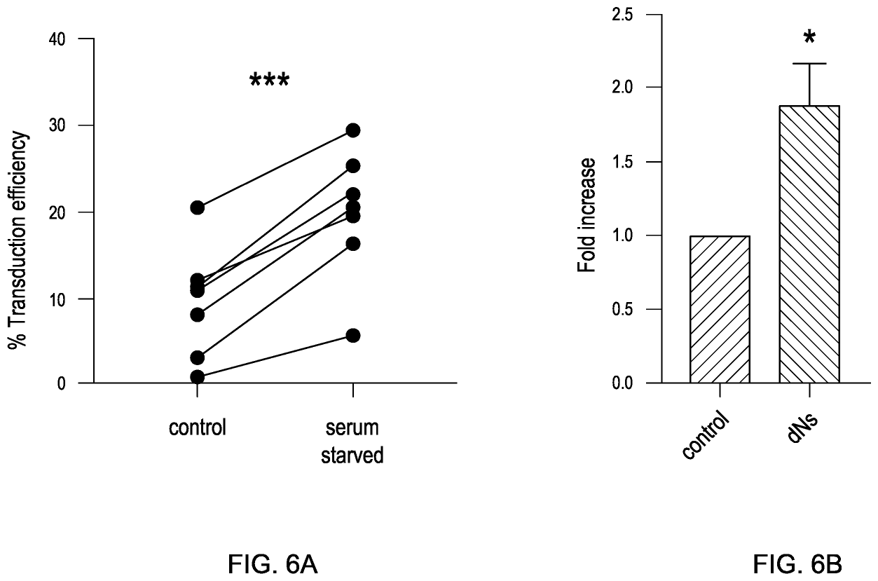

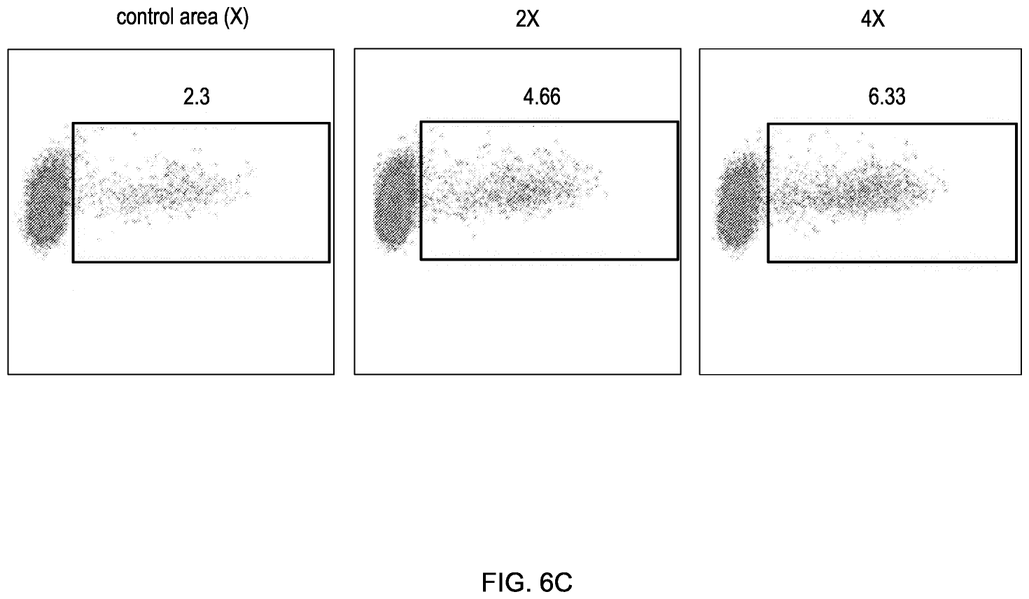

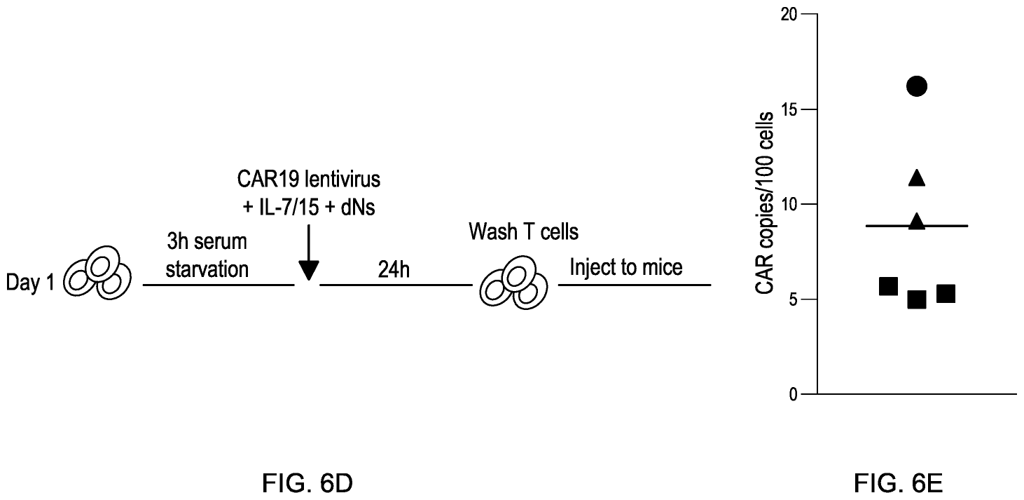

It was first evaluated whether a brief serum starvation prior to lentiviral vector transduction increases lentiviral vector transduction of non-activated T cells. 3 hours of serum starvation increases iRFP expression by an average of 2-fold in non-activated T cells (FIG. 6A).

The slow kinetics of reverse transcription in non-activated T cells by both natural HIV and lentiviral vectors also contribute to the reduced transduction efficiency. In was next shown that supplementing the culture medium with 50 μM dNs also increases lentiviral transduction of non-activated T cells by almost 2-fold (FIG. 6B).

Finally, the limited diffusibility of lentivirus in large culture vessels is another major barrier limiting transduction efficiency in T cells. To evaluate this, the geometric conditions were adjusted to enhance the colocalization of vector particles with T cells. By increasing the surface area to volume ratio of the culture vessel, while keeping the volume constant, transduction of non-activated T cells was increased by at least 2-fold (FIG. 6C).

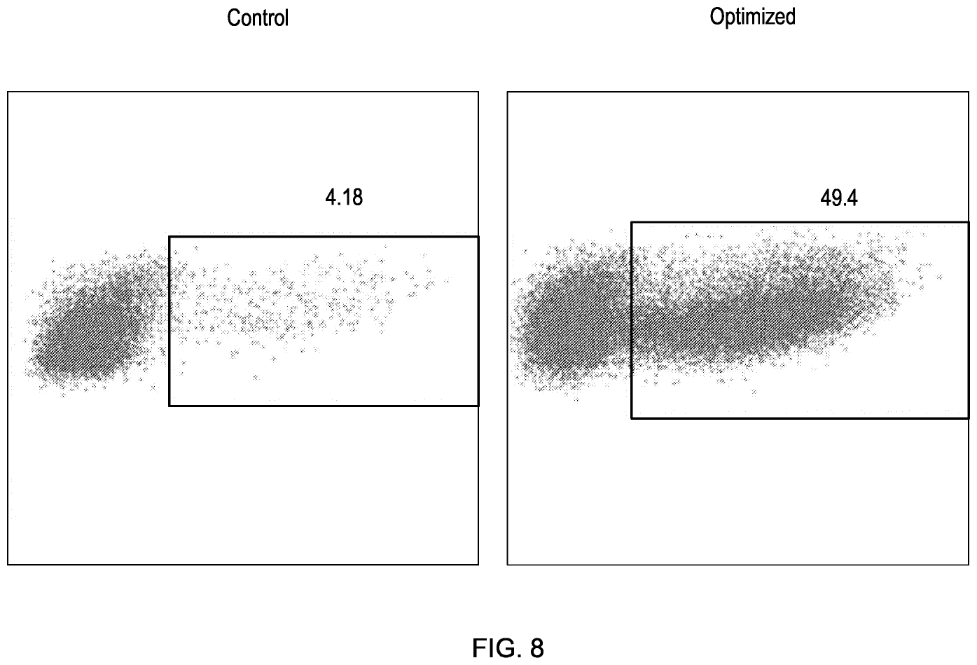

Combining these approaches, it was shown that the transduction efficiency of non-activated T cells maintained in IL-7 and IL-15 can be enhanced to achieve as high as 50% transduction (FIG. 8).

Since pseudotransduction with CD19-specific CAR interferes with the ability to estimate transduction efficiency (FIGS. 4A-4C) and vector integration is likely ongoing at the time of infusion, the efficiency of the optimized transduction process was estimated by adoptively transferring the transduced T cells into non-leukemia bearing NSG mice, which permits an estimation of the frequency of integrated vector in the absence of CAR stimulation that would normally enrich for CAR+ T cells. CART19 cells were generated from freshly isolated, peripheral blood T cells by serum starving the T cells for 3-hrs followed by transduction in a minimal volume of lentiviral vector encoding a CD19-specific CAR for 20 hours in the presence of 50 μM dNs, IL-7 and IL-15 at an MOI of 5 (FIG. 6D). Evaluating three CART19 products produced from three separate donors, a mean transduction frequency of 8% (range 6-16%) was observed based upon an integration analysis performed at 3 weeks following adoptive transfer (FIG. 6E), which is within the lower-end of the range of CART19 products using a 9-day manufacturing process (Maude et al., N Engl J Med. 2014; 371(16):1507-1517).

### In Vivo Functional Assessment of Optimally Transduced CD19-Specific CAR T Cells

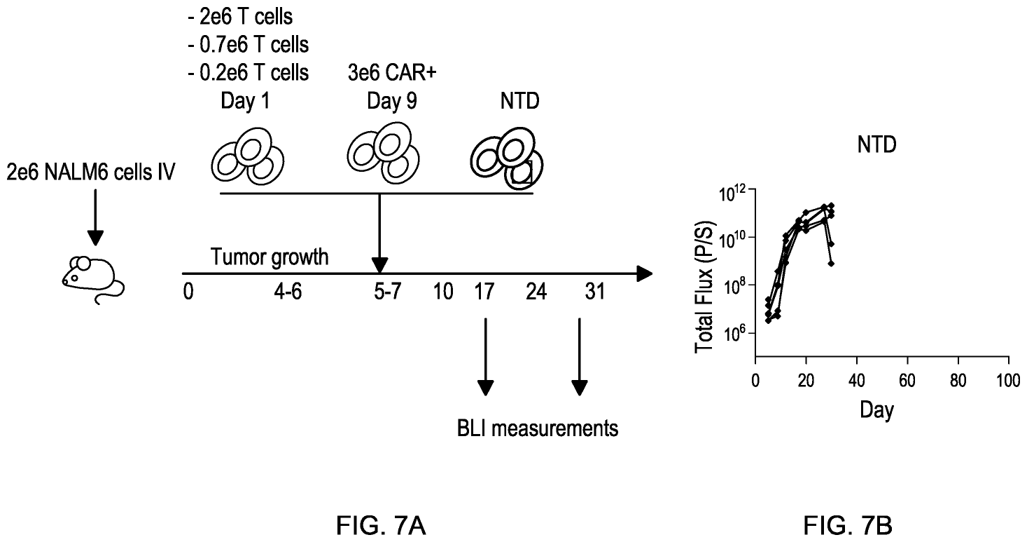

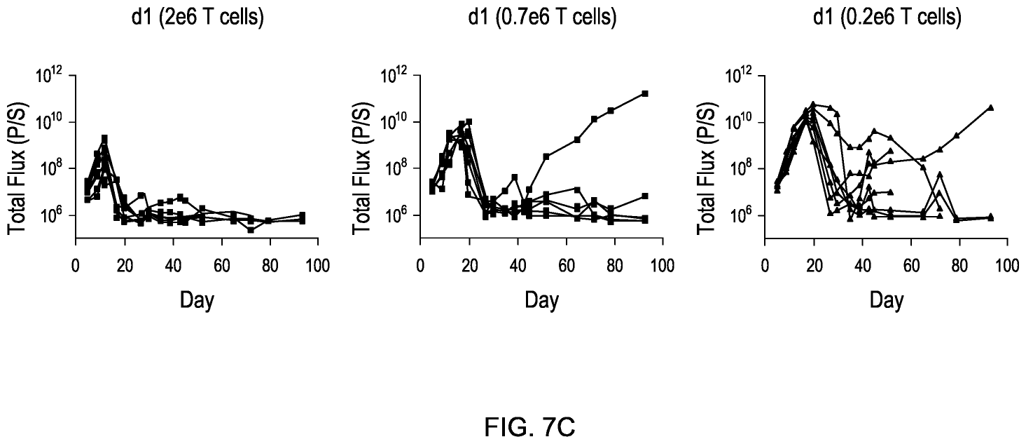

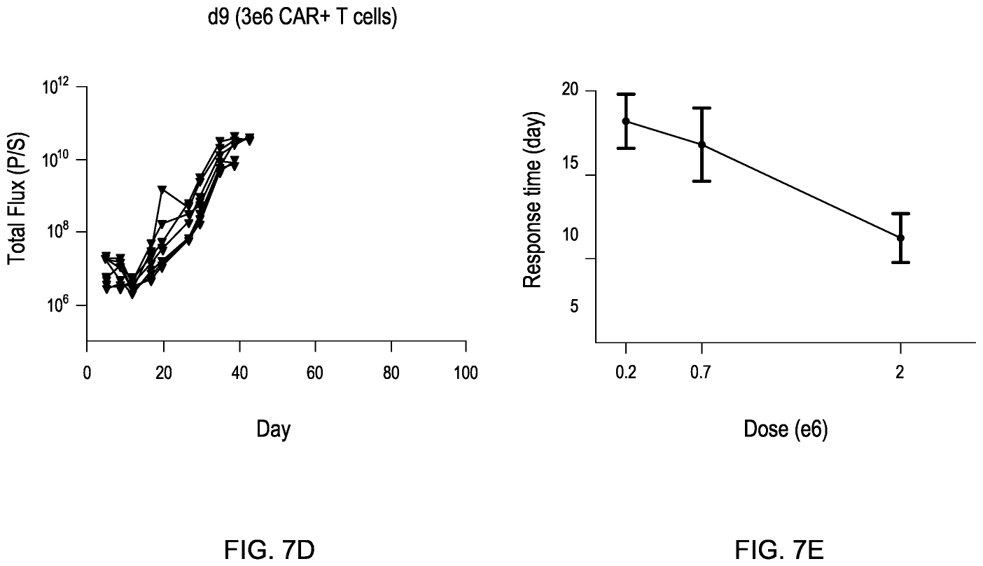

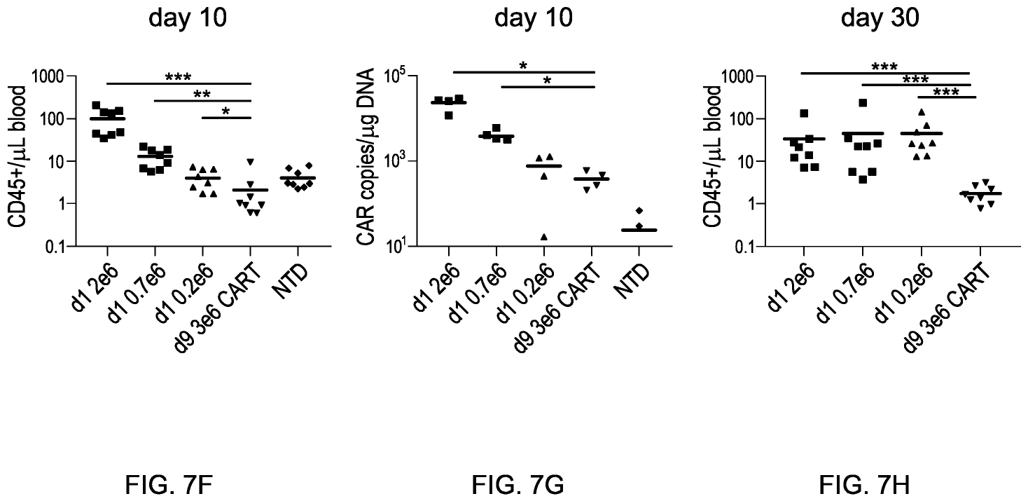

It was hypothesized that CAR T cells generated by transduction of non-activated T cells preserve the intrinsic stem-like properties of naïve and memory T cells by avoiding the differentiation induced by prior T cell activation. To evaluate this hypothesis, an in vivo functional “CAR stress test” was performed using a limited number of CAR T cells in the Nalm6 leukemia model. 2×10⁶, 7×10⁵ or 2×10⁵ total non-activated CART19 cells prepared using the optimized process described above were adoptively transferred into NSG mice bearing pre-established Nalm6 xenografts. 3×10⁶ activated CART19 cells prepared by anti-CD3/CD28 bead stimulation followed by 9 days of ex-vivo expansion was used as a control as well as 3×10⁶ mock-transduced, non-activated T cells (FIGS. 7A-7H). As shown in FIGS. 7B-7D, complete regression of Nalm6 leukemia was observed in all groups treated with CART19. The kinetics of the anti-leukemic response for the non-activated CART19 cells was dose dependent with a median time to complete response for the lowest dose group of 18 days compared with 11 days for the highest dose group (FIG. 7E). The CD3/CD28 stimulated and expanded CART19 cells showed the most rapid leukemia clearance (FIG. 7D). However, the durability of the response for this donor was limited with all mice relapsing by day 17. In contrast, the non-activated CART19 cells were able to control leukemia for the duration of the experiment (FIG. 7C). The durable control of leukemia with non-activated T cells was associated with improved persistence of T cells. This is demonstrated by the significantly increased absolute counts of CART19 cells in the peripheral blood of mice treated with non-activated CART19 cells, which varied in proportion to the dose infused, compared to treatment with activated CART19 cells (FIGS. 7F-7H). In summary, these findings demonstrate that as few as an estimated 12,000-32,000 non-activated CAR T cells, based upon the range of transduction efficiency generated with the optimized transduction process (FIG. 5E), within 24 hours from collection could eradicate leukemia. The long-term engraftment of these CAR T cells likely occurs due to their enhanced replicative capacity compared with activated CAR T cells.

### Discussion

This study presents a novel approach to rapidly generate highly functional CAR T cells for adoptive immunotherapy. This approach capitalizes upon the unique ability of lentiviral vectors to transduce non-activated, quiescent T cells. Currently, ex vivo cell culture following T cell activation is an essential part of the manufacturing of CAR T cell therapies. Because TCR activation and clonal expansion promotes irreversible differentiation of T cells as well as potentially other detrimental changes to the T cells through processes such as oxidative stress (Haining et al., Blood. 2005; 106(5):1749-1754; Halliwell et al., Biomed J. 2014; 37(3):99-105), the potency of CAR T cells may be compromised during their manufacturing process. While interventions including provision of different costimulatory receptor signals (Zhang et al., J Immunol. 2007; 179(7):4910-4918), cytokines (Klebanoff et al., Clin Cancer Res. 2011; 17(16):5343-5352), or other alterations to the culture conditions (e.g. Akt inhibition) (Klebanoff et al., JCI Insight. 2017; 2(23)) help to limit this cellular differentiation, stable expression of a CAR within a non-activated T cell provides a far simpler approach to limiting differentiation. The results demonstrate that functional transduction of non-activated T cells including memory subsets can be achieved within less than 24 hours of T cell collection. Moreover, these T cells transduced with a CD19-specific CAR exhibit potent in vivo anti-leukemic efficacy at cell doses well below those effective for activated T cells (Berger et al., J Clin Invest. 2008; 118(1):294-305).

The transduction methods described above can be further optimized. In some embodiments, the transduction method uses a microfluidic chamber described in Tran et al., Mol Ther. 2017; 25(10):2372-2382. This microfluidic chamber could significantly increase both the efficiency and kinetics of a transduction process by overcoming the diffusion barriers inherent in static cultures. In some embodiments, the transduction method comprises interference with SAMHD1, a deoxynucleoside triphosphate triphosphohydrolase that restricts HIV-1 infection in quiescent T cells (Descours et al., Retrovirology. 2012; 9:87; Baldauf et al., Nat Med. 2012; 18(11):1682-1687). Mechanistically, SAMHD1 hinders lentivirus infection by impeding the rate of reverse transcription. Loss-of-function approaches show that SAMHD1 elimination leads to increased infection efficiencies in non-activated T cells (Baldauf et al., Nat Med. 2012; 18(11):1682-1687). In some embodiments, the transduction method comprises using small molecules that inhibit SAMHD1, e.g., SAMHD1 small molecule inhibitors described in Mauney et al., Biochemistry. 2018; 57(47):6624-6636. In addition to the post-entry restrictions, substituting cocal virus envelope glycoprotein in place of VSV-G may also enhance the lentiviral entry step into quiescent T cells as this envelope protein has yielded superior transduction efficiencies in hematopoietic stem cells and activated T cells (Trobridge et al., Mol Ther. 2010; 18(4):725-733).

The slow kinetics of lentiviral transduction observed in this study complement the delay in reverse transcription and integration observed in natural HIV infection of non-activated CD4+ T cells (Naldini et al., Science. 1996; 272(5259):263-267; Plesa et al., J Virol. 2007; 81(24):13938-13942; Swiggard et al., J Virol. 2005; 79(22):14179-14188). It is likely that the majority of T cells used in the in vivo studies here lacked integrated proviral DNA at the time of adoptive transfer, and the processes of reverse transcription and integration instead likely occurred post-infusion. This introduces challenges to assessing CAR T cell quality. Transduction efficiency, typically measured by CAR expression at the cell surface and/or vector integration, is routinely used as one surrogate measure of product potency as well as CAR T cell dose. Translation of a rapid manufacturing approach using non-activated T cells will therefore require development of alternative methods to evaluate the transduction process such as by quantitation of reverse transcription intermediates.

In summary, the ability to generate highly functional CAR T cells within a day has important implications for improving CAR T cell therapies. Lentiviral vectors provide a highly efficient method to produce CAR T cell products with durable engraftment and function by leveraging the unique ability of these vectors to enter and integrate into the genome of non-dividing cells. Extended ex-vivo culture of T cells is unnecessary to produce CAR T cells for therapeutic purposes. Minimizing ex-vivo manipulation, in addition to reducing costs, conserves limited resources such as human serum and manufacturing space as T cell clonal expansion occurs entirely in vivo. If the process can be reduced to a few simple steps, it also has the potential to decentralize CAR T cell manufacturing to local hospital laboratories that are closer to the patient avoiding many of the logistical challenges. The generation of engineered T cell products within a shorter interval between apheresis collection and re-infusion of CAR T cells could also be of particular benefit to patients with rapidly progressive disease, who may otherwise be unable to receive the therapy (Couzin-Frankel et al., 2017; 356(6343):1112-1113).

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of making a population of immune cells (e.g., T cells) that express a chimeric antigen receptor (CAR), the method comprising:
(i) incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; and
(ii) transducing the population of immune cells with a nucleic acid molecule, e.g., a nucleic acid molecule on a lentiviral vector, encoding the CAR, in a medium comprising serum (e.g., at least about 4, 5, or 6% serum) and optionally deoxynucleosides (e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides), e.g., for about 14-30 hours, e.g., for about 14, 16, 18, 20, 22, 24, 26, or 28 hours, optionally wherein step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (ii) is performed at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody,
thereby expressing the CAR.

2. The method of claim 1, further comprising:
(iii) harvesting the population of immune cells for storage (e.g., reformulating the population of immune cells in cryopreservation media) or administration, wherein:
(a) step (iii) is performed no later than 48 hours, e.g., no later than 14, 16, 18, 20, 22, 24, 26, 28, 30, or 32 hours after the beginning of step (i),
(b) the population of immune cells from step (iii) is not expanded, or is expanded by no more than 10, 20, 30, 40, or 50%, e.g., no more than 10%, compared with the population of immune cells at the beginning of step (i),
(c) the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i), and/or
(d) the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i).

3. The method of claim 1 or 2, wherein step (i) comprises incubating the population of immune cells (e.g., T cells, e.g., freshly isolated T cells, e.g., freshly isolated quiescent T cells) in a medium that does not comprise serum for about 2-6 hours.

4. The method of any one of claims 1-3, wherein step (i) increases expression of low-density lipoprotein receptor (LDL-R) in the population of immune cells, e.g., increases expression of LDL-R by at least about 20, 40, 60, 80, 100, 500, or 1000%, compared with the expression of LDL-R in the population of immune cells prior to step (i).

5. The method of any one of claims 1-4, wherein step (i) increases transduction efficiency of step (ii) by, e.g., at least about 2, 4, 6, 8, 10, or 12-fold, compared with an otherwise similar method without step (i), e.g., as measured by expression of CAR (e.g., the percentage of CAR-expressing cells) in the population of immune cells at the end of step (ii).

6. The method of any one of claims 1-5, wherein step (ii) comprises transducing the population of immune cells with a nucleic acid molecule, e.g., a nucleic acid molecule on a lentiviral vector, encoding the CAR in a medium comprising at least about 4, 5, or 6% serum.

7. The method of any one of claims 1-6, wherein step (ii) comprises transducing the population of immune cells with a lentiviral vector comprising a nucleic acid molecule encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours.

8. The method of claim 7, wherein step (ii) comprises transducing the population of immune cells with a lentiviral vector comprising a nucleic acid molecule encoding the CAR in a medium comprising about 50 μM deoxynucleosides for about 16-24 hours.

9. The method of claim 7 or 8, wherein transducing the population of immune cells in a medium comprising deoxynucleosides, e.g., at least about 40 μM-1.5 mM deoxynucleosides, increases transduction efficiency of step (ii) by, e.g., at least about 1, 2, 3, 4, or 5-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (ii).

10. The method of any one of claims 1-9, wherein step (ii) is performed in a medium comprising IL-7 (e.g., about 10 ng/mL of IL-7) and/or IL-15 (e.g., about 10 ng/mL of IL-15).

11. A method of making a population of immune cells (e.g., T cells) that express a chimeric antigen receptor (CAR), the method comprising:
(1) transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, optionally wherein step (1) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (1) is performed at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody,
thereby expressing the CAR.

12. The method of claim 11, further comprising:
(2) harvesting the population of immune cells for storage (e.g., reformulating the population of immune cells in cryopreservation media) or administration, wherein:
(a) step (2) is performed no later than 30 hours, e.g., no later than 12, 14, 16, 18, 20, 22, 24, 26, or 28 hours after the beginning of step (1),
(b) the population of immune cells from step (2) is not expanded, or is expanded by no more than 10, 20, 30, 40, or 50%, e.g., no more than 10%, compared with the population of immune cells at the beginning of step (1),
(c) the percentage of naïve cells, e.g., naïve T cells, in the population of immune cells from step (2) is not reduced, or is reduced by no more than 10, 20, or 30%, compared with the percentage of naïve cells, e.g., naïve T cells, in the population of immune cells at the beginning of step (1), and/or
(d) the percentage of differentiated cells, e.g., differentiated T cells, e.g., terminally differentiated T cells, e.g., CCR7^{low} T cells, in the population of immune cells from step (2) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (1).

13. The method of claim 11 or 12, wherein step (1) comprises transducing the population of immune cells (e.g., T cells, e.g., freshly isolated T cells, e.g., freshly isolated quiescent T cells) with a lentiviral vector comprising a nucleic acid molecule encoding the CAR in a medium comprising about 50 μM deoxynucleosides for about 16-24 hours.

14. The method of any one of claims 11-13, wherein transducing the population of immune cells in a medium comprising deoxynucleosides, e.g., at least about 40 μM-1.5 mM deoxynucleosides, increases transduction efficiency of step (1) by, e.g., at least about 1, 2, 3, 4, or 5-fold, compared with an otherwise similar method in which the population of immune cells is transuded in a medium that does not comprise deoxynucleosides, e.g., as measured by expression of CAR (e.g., the percentage of CAR-expressing cells) in the population of immune cells at the end of step (1).

15. The method of any one of claims 11-14, wherein step (1) is performed in a medium comprising IL-7 (e.g., about 10 ng/mL of IL-7) and/or IL-15 (e.g., about 10 ng/mL of IL-15).

16. The method of any one of claims 1-15, wherein the population of cells from step (iii) or step (2), after being administered in vivo, persists longer, expands at a higher level, and/or exhibits anti-tumor activity for a longer period, compared with cells made by an otherwise similar method in which cells are expanded in vitro for at least 6, 7, 8, 9, 10, 11, or 12 days before harvesting.

17. The method of any one of claims 1-16, wherein the CAR comprises an antigen binding domain, a transmembrane domain, and an intracellular signaling domain.

18. The method of claim 17, wherein the antigen binding domain binds to an antigen chosen from: CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRCSD, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

19. The method of claim 17 or 18, wherein the antigen binding domain comprises a CDR, VH, VL, scFv or a CAR sequence disclosed herein.

20. The method of any one of claims 17-19, wherein:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

21. The method of any one of claims 17-20, wherein the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

22. The method of any one of claims 17-21, wherein the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FccRI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

23. The method of any one of claims 17-22, wherein the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

24. The method of any one of claims 17-23, wherein the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

25. The method of any one of claims 17-24, wherein the CAR further comprises a leader sequence comprising the amino acid sequence of SEQ ID NO: 1.

26. A population of CAR-expressing cells (e.g., autologous or allogeneic CAR-expressing T cells or NK cells) made by the method of any one of claims 1-25.

27. A pharmaceutical composition comprising the population of CAR-expressing cells of claim 26 and a pharmaceutically acceptable carrier.

28. A method of increasing an immune response in a subject, comprising administering the population of CAR-expressing cells of claim 26 or the pharmaceutical composition of claim 27 to the subject, thereby increasing an immune response in the subject.

29. A method of treating a cancer in a subject, comprising administering the population of CAR-expressing cells of claim 26 or the pharmaceutical composition of claim 27 to the subject, thereby treating the cancer in the subject.

30. A method of treating a cancer in a subject, comprising administering a population of immune cells expressing a CAR to the subject, thereby treating the cancer in the subject, wherein the population of immune cells expressing a CAR was obtained by:
(i) incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 4, 6, 8, or 10 hours; and
(ii) transducing the population of immune cells with a nucleic acid molecule, e.g., a nucleic acid molecule on a lentiviral vector, encoding the CAR, in a medium comprising serum (e.g., at least about 4, 5, or 6% serum) and optionally deoxynucleosides (e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides), e.g., for about 14-30 hours, e.g., for about 14, 16, 18, 20, 22, 24, 26, or 28 hours, optionally wherein step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (ii) is performed at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody,
thereby generating the population of immune cells expressing a CAR.

31. A method of treating a cancer in a subject, comprising administering a population of immune cells expressing a CAR to the subject, thereby treating the cancer in the subject, wherein the population of immune cells expressing a CAR was obtained by:
(1) transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, optionally wherein step (1) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., step (1) is performed at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody, thereby generating the population of immune cells expressing a CAR.

32. The method of claim 30 or 31, wherein the population of immune cells expressing a CAR was obtained from a third party.

33. A method of treating a cancer in a subject, comprising:
incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 1.4, 1.6, 1.8, or 2% serum, e.g., for at least about 1-10 hours, e.g., for at least about 2, 4, 6, 8, or 10 hours,
transducing the population of immune cells with a nucleic acid molecule, e.g., a nucleic acid molecule on a lentiviral vector, encoding a CAR, in a medium comprising serum (e.g., at least about 4, 5, or 6% serum) and optionally deoxynucleosides (e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides), e.g., for about 14-30 hours, e.g., for about 14, 16, 18, 20, 22, 24, 26, or 28 hours, optionally wherein the population of immune cells is transduced at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody,
thereby generating a population of immune cells expressing a CAR; and
administering the population of immune cells expressing a CAR to the subject, thereby treating the cancer in the subject.

34. A method of treating a cancer in a subject, comprising:
transducing a population of immune cells (e.g., T cells, e.g., freshly isolated T cells, e.g., freshly isolated quiescent T cells) with a nucleic acid molecule, e.g., a nucleic acid molecule on a lentiviral vector, encoding the CAR in a medium comprising deoxynucleosides, e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides, e.g., for about 14-30 hours, e.g., for about 14, 16, 18, 20, 22, 24, 26, or 28 hours, optionally wherein the population of immune cells is transduced at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g., at a cell concentration of about 1×10⁷ cells/mL,
wherein the population of immune cells is not contacted in vitro with an agent that stimulates a CD3/TCR complex and/or an agent that stimulates a costimulatory molecule, e.g., anti-CD3 antibody and/or anti-CD28 antibody, thereby generating a population of immune cells expressing a CAR; and
administering the population of immune cells expressing a CAR to the subject, thereby treating the cancer in the subject.

35. The method of any one of claims 29-34, wherein the cancer is a solid cancer, e.g., chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

36. The method of any one of claims 29-34, wherein the cancer is a liquid cancer, e.g., chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.


SEQUENCE LISTING (126 sequences)

SEQ ID NO: 1 - protein, 21 aa, Artificial Sequence
  source: /note=“Description of Artificial Sequence: Synthetic peptide”
MALPVTALLLPLALLLHAARP

SEQ ID NO: 2 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 3 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, Artificial Sequence
  source: /note=“Description of Artificial Sequence: Synthetic peptide”
GGGGSGGGGS

SEQ ID NO: 6 - protein, 24 aa, Artificial Sequence
  source: /note=“Description of Artificial Sequence: Synthetic peptide”
IYIWAPLAGTCGVLLLSLVITLYC

SEQ ID NO: 7 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 8 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 9 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 13 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, Artificial Sequence
  source: /note=“Description of Artificial Sequence: Synthetic oligonucleotide”
ggtggcggaggttctggaggtggaggttcc

SEQ ID NO: 17 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 18 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 24 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 5 aa, Artificial Sequence
  source: /note=“Description of Artificial Sequence: Synthetic peptide”
GGGGS

SEQ ID NO: 26 - protein, 30 aa, Artificial Sequence
  source: /note=“Description of Artificial Sequence: Synthetic polypeptide”
  SITE (1)..(30): /note=“This sequence may encompass 1-6 ‘Gly Gly Gly Gly Ser’ repeating units”
GGGGSGGGGSGGGGSGGGGSGGGGSGGGGS

SEQ ID NO: 27 - protein, 20 aa, Artificial Sequence
  source: /note=“Description of Artificial Sequence: Synthetic peptide”
GGGGSGGGGSGGGGSGGGGS

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 32 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, Artificial Sequence; identical to SEQ ID NO: 30
  source: /note=“Description of Artificial Sequence: Synthetic polynucleotide”
  misc_feature (1)..(5000): /note=“This sequence may encompass 100-5000 nucleotides”

SEQ ID NO: 34 - RNA, 400 nt, Artificial Sequence
  source: /note=“Description of Artificial Sequence: Synthetic polynucleotide”
  misc_feature (1)..(400): /note=“This sequence may encompass 100-400 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 37 - DNA, 123 bp, Artificial Sequence; identical to SEQ ID NO: 19
  source: /note=“Description of Artificial Sequence: Synthetic polynucleotide”

SEQ ID NO: 38 - protein, 35 aa, Artificial Sequence
  source: /note=“Description of Artificial Sequence: Synthetic polypeptide”
TKKKYSSSVHDPNGEYMFMRAVNTAKKSRLTDVTL

SEQ ID NO: 39 - DNA, 105 bp, Artificial Sequence
  source: /note=“Description of Artificial Sequence: Synthetic polynucleotide”
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, Artificial Sequence
  source: /note=“Description of Artificial Sequence: Synthetic oligonucleotide”
ggtggcggaggttctggaggtgggggttcc

SEQ ID NO: 41 - protein, 4 aa, Artificial Sequence
  source: /note=“Description of Artificial Sequence: Synthetic peptide”
  source: /note=“See specification as filed for detailed description of substitutions and preferred embodiments”
GGGS

SEQ ID NO: 42 - protein, 40 aa, Artificial Sequence
  source: /note=“Description of Artificial Sequence: Synthetic polypeptide”
  SITE (1)..(40): /note=“This sequence may encompass 1-10 ‘Gly Gly Gly Ser’ repeating units”
GGGSGGGSGGGSGGGSGGGSGGGSGGGSGGGSGGGSGGGS

SEQ ID NO: 43 - protein, 18 aa, Artificial Sequence
  source: /note=“Description of Artificial Sequence: Synthetic peptide”
GSTSGSGKPGSGEGSTKG

SEQ ID NO: 44-107 - skipped (64 entries)

SEQ ID NO: 108 - protein, 1132 aa, Homo sapiens [sequence omitted]

SEQ ID NO: 109 - DNA, 521 bp, Unknown
  source: /note=“Description of Unknown: PGK promoter sequence”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 110 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 111 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 112 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 113 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 114 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 115 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 116 - protein, 93 aa, Artificial Sequence [sequence omitted]

SEQ ID NO: 117 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 118 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 119 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 120 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 121 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 122 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 123 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 125 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 5 aa, Artificial Sequence
  source: /note=“Description of Artificial Sequence: Synthetic peptide”
  source: /note=“See specification as filed for detailed description of substitutions and preferred embodiments”
GGGGS